(19) United States
(12) Patent Application Publication
(10) Pub. No.: US 2025/0296986 A1
(43) Pub. Date: Sep. 25, 2025

(54) ENGINEERED POLYPEPTIDES

(71) Applicant: Vir Biotechnology, Inc., San Francisco, CA (US)

(72) Inventors: Istvan BARTHA, Bellinzona (CH); Davide CORTI, Bellinzona (CH); Nadine CZUDNOCHOWSKI, San Francisco, CA (US); Michael Alexander SCHMID, Bellinzona (CH); Gyorgy SNELL, San Francisco, CA (US); Amalio TELENTI, San Francisco, CA (US)

(21) Appl. No.: 18/563,674

(22) PCT Filed: May 23, 2022
(86) PCT No.: PCT/US2022/030556

§ 371 Date: Nov. 22, 2023

Related U.S. Application Data
(60) Provisional application No. 63/192,549, filed on May 24, 2021.
(60) Provisional application No. 63/265,032, filed on Dec. 6, 2021.
(60) Provisional application No. 63/266,453, filed on Jan. 5, 2022.

(51) Int. Cl. C07K 16/10; A61K 39/00; A61P 37/04

(52) U.S. Cl. CPC C07K 16/1018; A61P 37/04; A61K 2039/505; C07K 2317/41; C07K 2317/52; C07K 2317/92

(57) ABSTRACT

Provided herein are engineered polypeptides (e.g., Fc polypeptides, Fc polypeptide fragments, Fc fusion proteins, antibodies, and the like) that comprise a variant of an IgG Fc polypeptide (or a portion or fragment thereof), which variants (and the polypeptides that comprise these variants) have one or more improved characteristics over known Fc polypeptides.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is SeqList3_368564_42201.txt. The text file is 77,931 bytes, was created on Jun. 14, 2024, and is being submitted electronically.

### BACKGROUND

Therapies involving antibodies and other molecules that include immunoglobulin Fc domains are developing. Fcs can interact with immune system proteins such as FcγRs and complement C1q, and the nature of such interactions can provide for different outcomes, such as, for example, activating or suppressing a host immune response against a pathogen.

### BRIEF DESCRIPTION OF THE SEVERAL VIEWS OF THE DRAWINGS

FIGS. 1A-1E show that antibodies with certain Fc modifications have improved prophylactic effect against influenza A. FIG. 1A shows the design of a study in which mice engineered to express human FcγRs were intravenously administered antibodies bearing variant Fcs two days prior to infection with a lethal intranasal dose of H1N1 PR8. Serum IgG levels were evaluated at the time of infection (day 0) and mice were evaluated for body weight and survival over fourteen days. FIG. 1B shows maximum change in body weight in mice pre-treated with anti-FluA IgG1 antibody “F18” bearing G236A/A330L/I332E/M428L/N434S (“F18-LS-GAALIE”) Fc mutations compared with mice pre-treated with F18 antibody bearing only M428L/N434S mutations in the Fc (“F18-LS”). Body weight change (FIG. 1C) and survival (FIG. 1D) were also evaluated in mice treated with antibody bearing G236A (“GA”), A330L/I332E (“ALIE”), G236A/A330L/I332E (“GAALIE”), or G237D/H268D/P271G/A330R (“V11”) Fc mutations. Mice that were treated with antibody bearing wild-type Fc or afucosylated wild-type Fc, or treated with PBS, were evaluated as controls. The effect of the Fc mutation(s) on antibody binding to FcγRI, FcγRIIa, and FcγRIIb, and on the ratio of FcγRIIIa to FcγRIIIb binding, is shown in the legend of FIG. 1C. FIG. 1E summarizes results of binding to various FcγRs by the indicated Fc variants.

FIG. 2 shows predicted binding affinity of certain Fc variant antibodies to FcγRIIa (R131 allele) and FcγRIIb.

FIG. 3 shows FcγR and C1q binding affinity (measured by a meso scale discovery-based assay (MSD; employing electrochemiluminescence)) and other characteristics of certain IgG1 Fc variant antibodies. Fc variants shown beginning in the third row down (“G236A_E272Y_S298N” and below) were identified using an iterative discovery workflow. The G236A_A330L_I332E variant was used as a comparator. Binding of Fc variant antibodies to FcγRIIA-H (high affinity H158 allele), FcγRIIB, FcγRIIA-R (low affinity R131 allele), FcγRIIIA-V (high affinity V158 allele), FcγRIIIA-F (low affinity F158 allele), FcγRIIIB and FcRn was tested. Data are reported as fold-change in binding compared to wild-type IgG1. The ratio of FcγRIIA/FcγRIIB binding, as well as production titer (mg/mL) and Tm (° C.) relative to wild-type IgG1, are also shown.

FIGS. 4A-4C show effect of fucosylation on production and purification of twenty Fc variant antibodies. Variants were expressed in the absence (“No 2FF”) or presence (“+2FF”) of 2-deoxy-2-fluoro-L-fucose (2FF); 2FF reduces fucosylation. FIG. 4A shows antibody titers as determined using a Protein A column. FIG. 4B shows yields resulting from two replicate purifications. The table in FIG. 4C summarizes the theoretical maximum yield and average yield, both measured in pg, along with the calculated average recovery and protein concentration of the second elution (measured in pg/ml). Fc variants were purified using two elutions and combined prior to determining yield.

FIG. 5 shows representative absolute size exclusion chromatography analysis of purified Fc variant antibodies. The single peak at left was typical of tested variants, while the double-peak at right shows a variant for which low molecular weight species (LMWS) were observed.

FIGS. 6A and 6B show Tm curves of antibodies with wild-type (6A) or R292P variant Fc (6B).

FIGS. 7A and 7B summarize FcγR binding and other characteristics of Fc variants, relative to wildtype Fc. Bars and values indicate fold-change in binding as compared with wild-type Fc. Fc variants shown were not treated with 2FF. FIG. 7A shows binding to FcγRIIA-H (high affinity), FcγRIIA-R (low affinity), FcγRIIB, FcγRIIIA-V (high affinity), FcγRIIIA-F (low affinity), and FcRn (at pH 6). FIG. 7B further shows the ratio of FcγRIIA-H/FcγRIIB binding, as well as C1q binding and complement-dependent cytotoxicity (CDC), with the WT “baseline” value indicated by a dashed vertical red line. Binding was measured by a meso scale discovery-based assay (MSD; employing electrochemiluminescence)).

FIG. 8 shows binding of certain Fc variants to FcγRIIA-H (high affinity) and FcγRIIB. Plots connected by a line represent the same variant. Variants shown were not treated with 2FF.

FIGS. 9A-9B shows FcγR signaling through different FcγRs as measured using a reporter cell assay (Promega; tested cells expressed one type/allele FcγR, as indicated). Fc variants shown are fucosylated (“fuc”; 9A/9B) or afucosylated (“afuc”; 9B) as indicated in the figure. Values are calculated from an average of three experiments and indicate fold-change (expressed linear) in area-under-the-curve (plotted in log) as compared to wildtype Fc.

FIGS. 10A-1-10C summarize characteristics of certain variant Fcs. Antibodies comprising the indicated Fc were expressed as recombinant human IgG1. Variants shown in FIGS. 10B-1-10B-4 are afucosylated. Binding was measured by a meso scale discovery-based assay (MSD; employing electrochemiluminescence)). Values represent fold-change compared to the antibody comprising wild-type fucosylated human IgG1 Fc. FIGS. 10A-3, 10A-4, 10B-3, and 10B-4 also show fold-change in FcγR signaling, as measured using a reporter cell assay.

FIG. 11 shows (left) a schematic of a Meso Scale Discovery binding assay for assessing binding of a Fc variant antibody to a FcγR and (right) a schematic of a cell reporter assay for measuring FcγR-mediated cell signaling induced by an Fc variant antibody.

FIGS. 12A-12B show FcγR signaling through FcγRIIA-H (high affinity, FIG. 12A) and FcγRIIB (FIG. 12B) by the Fc variant “G236A_R292P_Y300L” as measured using a reporter cell assay.

FIGS. 13A-13B show FcγR binding versus signaling through FcγRIIA-H (high affinity, FIG. 13A) and FcγRIIB (FIG. 13B) by Fc variants. FcγR binding was measured using a Meso Scale Discovery binding assay and FcγR signaling was measured using a reporter cell assay (Promega).

FIGS. 14A-14B show activation, by anti-flu HA antibody FM08 containing variant Fc, of Jurkat cells expressing human FcγRIIA (H131) (A) or FcγRIIIA (F158) (B) with A549-CA cell line stably expressing HA of influzena CA-2009-H1N1. FIG. 14C shows NK cell-mediated ADCC by the antibodies against target A549-CA cells stably expressing HA of influzena CA-2009-H1N1 and using isolated effector NK cells (HM_WB014_FF) cells at E:T ratio of 6:1, in a LDH release assay.

FIG. 15 summarizes results from C1q binding assays using the indicated FY1 (anti-flu hemaggluttinin (“HA”) stem; Kallewaard et al. Cell 166(3):596-608 (2016)) Fc variant antibodies. Assay: Binding of antibody Fc (random capture on Octet sensor) to human C1q in solution.

FIGS. 16A-16B show activation, by anti-HBsAg antibody HBC34v35 containing variant Fc, of Jurkat cells expressing human FcγRIIA (H131) (A) or FcγRIIIA (F158) (B) with target cells line stably expressing HBsAg.

FIGS. 17A-17D show results from repeat experiments of those in FIGS. 16A and 16B.

In FIGS. 18-20, the following acronyms are used for Fc variants: GA=G236A; GALVQE=G236A L328V_Q295E; GAYL=G236A_Y300L; GARPYL=G236A_R292P_Y300L; and GARPIN=G236A_R292P_I377N; GAALIE=G236A_A330L_I332E; GRLR=G236R_L328R.

FIG. 18 shows results from experiments measuring: FcγR-binding; ratio of binding of FcγRIIA alleles to FcγRIIB; C1q-binding; melting temperature; and FcRn-binding, by certain Fc variant antibodies. Anti-influenza antibody FY1 was expressed as recombinant IgGlm3 with M428L and N434S mutations in CH3, and with the indicated combination mutations elsewhere in the Fc. Binding (one study) was measured by a meso scale discovery-based assay (MSD; employing electrochemiluminescence)). Binding data are shown as fold-change relative to FY1 rIgGlm3-MLNS without the other Fc mutations. FcγR-binding was confirmed by FcγR-signaling using a reporter cell assay (NFAT driving luciferase) (Promega).

FIG. 19 shows results from additional experiments measuring the antibody features as in FIG. 18. In these experiments, FY1 was expressed as recombinant IgGlm3 without the M428L and N434S mutations (i.e., with wild-type IgGlm3 CH1-CH3 or with the mutations indicated in the table). The FY1-rIgGlm3 and FY1-rIgGlm3-GAALIE antibodies were produced and measured twice independently in a first plate; averaged data is shown. FY1-rIgGlm3-GA antibody was produced 2× independently in a first and a second plate. For the other variants, a single measurement was performed. Binding was measured by a meso scale discovery-based assay (MSD; employing electrochemiluminescence)). Binding data are shown as fold-change relative to FY1 rIgGlm3 with wild-type Fc. FcγR-binding/activation was using a reporter cell assay (NFAT driving luciferase) (Promega).

FIG. 20 shows results from additional experiments measuring features as in FIG. 19, using afucosylated Fc variant antibodies. Antibodies were produced in the presence of 2FF to obtain afucosylated glycans. In these experiments, FY1 was expressed as recombinant IgGlm3 without the M428L and N434S mutations (i.e., with wild-type IgGlm3 CH1-CH3 or with the mutations indicated in the table). The FY1-rIgGlm3 and FY1-rIgGlm3-GAALIE antibodies were produced and measured twice independently in a first plate; averaged data is shown. FY1-rIgGlm3-GA antibody was produced 2× independently in a first and a second plate. For the other variants, a single measurement was performed. Binding was measured by a meso scale discovery-based assay (MSD; employing electrochemiluminescence)).

FIG. 21 shows FcγRIIA activation/signaling by anti-influenza FY1 antibodies with variant Fc, as indicated in the key. Target cells were A549 cells expressing FluA H1N1 HA, and reporter cells were Jurkat cells expressing FcγRIIA (H131 allele) and luciferase under control of a NFAT promoter.

FIGS. 22A and 22B shows FcγRIIIA activation/signaling by anti-influenza FY1 antibodies with variant Fc, as indicated in the key. Target cells were A549 cells expressing FluA H1N1 HA, and reporter cells were Jurkat cells expressing FcγRIIIA (F158 lower-affinity allele (A) or V158 higher-affinity allele (B)) and luciferase under control of a NFAT promoter.

FIG. 23 shows a schematic illustrating a surface plasmon resonance assay for measuring binding kinetics of FY1 Fc variants (expressed as recombinant rIgGlm3 with M428L and N434S and no other Fc mutations, or with the indicated further Fc mutations) against human FcγRs. Briefly, a CAP chip was used to capture biotinylated FcγRs by streptavidin. FY1 Fc variants were injected at concentrations of 819, 273, 91, 30.3, and 10.1 nM. Injections performed successively with no regeneration between different concentrations of the same sample. Injection: 600 seconds. Dissociation 100 seconds each injection.

FIG. 24 provides a table showing fold-change results (vs. the reference FY1-rIgGlm3-LS antibody) from SPR binding data. N=1. Fold changes were calculated by dividing the affinity value determined for FY1-rIgGlm3-LS with the value determined for the Fc variant. A larger fold-change represents a decrease in KD and increase in affinity. “-” =no measurable binding or weak binding.

FIGS. 25A-25B show FcγR activation/signaling by anti-SARS-CoV-2 antibody S309 with variant Fc as indicated in the keys. All antibodies except the negative control “S309-GRLR” (including G236R and L328R Fc mutations) included M428L and N434S Fc mutations. Activation/signaling was measured using CHO cells expressing SARS-CoV-2 S protein and luciferase reporter cells (Promega) expressing FcγRIIIA (A) or FcγRIIA (B).

FIG. 25C shows NK cell-mediated ADCC by 5309 Fc variant antibodies. Donor PBMCs and S-CHO-HiBit cells were used, as indicated. Taking the data points at 10⁴ ng/mL antibody concentration, the curves in FIG. 25A are as follows, from top to bottom: 5309-LS-afuc; S309-LS-GA-afuc; S309-LS-GAPAQE-afuc; S309-LS-GALVQE-afuc S309-LS-GAALIE˜S309-LS-GARPYL; S309-LS; S309-LS-GAYL; S309-LS-GA; S309-LS-GAPAQE˜S309-LS-GALVQE˜S309-GRLR. At the 10⁴ ng/mL antibody concentration in FIG. 25B, the bottom curve is S309-GRLR, the second-from-bottom curve is S309-LS, and the third-from-bottom curve is S309-LS-GAALIE. In FIG. 25C, some Fc variants (5309-LS-afuc; S309-LS-GA-afuc; S309-LS-GARPYL; S309-LS-GALVQE-afuc; S309-LS-GAALIE) did not titrate on ADCC as the signal achieved approximated the maximum/plateau of the assay. For the other Fc variants, the ranking was as follows: 5309-LS-GAPAQE-afuc>S309-LS>S309-LS-GA>S309-LS-GAYL>S309-LS-GALVQE>S309-LS-GAPAQE>S309-GRLR.

FIGS. 26A-26F show FcγR signaling (A-D) and NK-cell mediated killing (E-F) induced via 5309 Fc variants. (A) and (B) show FcγRIIa activation/signaling using Jurkat reporter cells (Promega) expressing FcγRIIa (H131 allele) driving expression of luciferase and, as target cells, CHO expressing SARS-CoV-2 spike protein. (A)=fucosylated antibodies, (B)=afucosylated antibodies. (C) and (D) show FcγRIIIa activation/signaling using Jurkat reporter cells (Promega) expressing FcγRIIIa (V158 allele) driving expression of luciferase and, as target cells, CHO expressing SARS-CoV-2 spike protein. (C)=fucosylated antibodies, (D)=afucosylated antibodies, except for S309-LS and S309-GRLR comparators. (E) and (F) show NK cell-mediated ADCC. PBMCs/NK donor cells expressing FcγRIIIa (heterozygous V158/F158) and, as target cells, CHO expressing SARS-CoV-2 spike protein, were used. (E)=fucosylated antibodies, (F)=afucosylated antibodies, except for S309-LS and S309-GRLR comparators. Some Fc variants did not titrate on ADCC as the signal achieved approximated the maximum/plateau of the assay. In FIG. 26E, the lowest curve corresponds to 5309-GRLR. In FIG. 26F, the curve with the second-lowest data point at 10⁴ ng/mL antibody corresponds to 5309-LS.

FIGS. 27A-27J relate to certain anti-SARS-CoV-2 antibodies comprising variant Fcs. (A)-(C): FcγRIIIA activation/signaling as determined by luminescence at 23h using reporter cells expressing human FcγRIIIA driving luciferase expression and, as target cells, ExpiCHO transfected with SARS-CoV-2 spike protein as indicated; in (A), the highest curve corresponds to S309; in (B), mutation-stabilized Wuhan-Hu-1 spike protein, in which shedding of S protein from the target cell is not possible, was used. (D)-(F): FcγRIIA activation/signaling as determined by luminescence at 23h using reporter cells expressing human FcγRIIA driving luciferase expression and, as target cells, ExpiCHO transfected with SARS-CoV-2 spike protein as indicated. (G)-(H): NK cell-mediated ADCC (2 NK cell donors, one expressing FcγRIIIA F158/V158 (G) and the other expressing V158/V158 (H)) using S-CHO-HiBiT cells (expressing wild-type SARS-CoV-2 Wuhan-Hu-1 spike sequence) as target cells, as indicated. (I)-(J) monocyte-mediated ADCP (2 monocyte donors, one expressing FcγRIIA R131/H131 and FcγRIIIA F158/F158 (I) and the other expressing FcγRIIA R131/H131 and FcγRIIIA F158/V158 (J)) using CHO cells expressing SARS-CoV-2 Wuhan Spike protein, as indicated. The dashed horizontal lines at/near the bottom of each graph indicate the lysis value for target cells+effector cells without antibody. In FIG. 27B at the 10⁴ ng/mL antibody concentration, the curves are, from top to bottom: S2X259-LS-GA-afuc; S2X259-v5 GAALIE; S2X259-LS-GARPYL; S309; S2X259-LS; S2X259-LS-GA; S2X259-LS-GALVQE˜S2X259-LS-GALVQE-afuc˜S309-GRLR˜S2X259-GRLR. In FIG. 27C at the 10⁴ ng/mL antibody concentration, the curves are, from top to bottom: S309; S2X259-LS-GALVQE-afuc; S2X259-LS-GRLR; S2X259-LS-GARPYL; S2X259-LS-GA; S2X259-v5-GAALIE; S2X259-LS; S2X259-LS-GALVQE; S309-GRLR. In FIG. 27E at the 10⁴ ng/mL antibody concentration, the top five curves are, from top to bottom: S2X259-LS-GALVQE; S2X259-LS-GA; S2X259-LS-GARPYL; S2X259-v5-GAALIE; S2X259-LS-GA-afuc. In FIG. 27G at the 10⁴ ng/mL antibody concentration, the curves are, from top to bottom: S2X259.1-LS-GARPYL; S2X259.1-LS-GALVQE-afuc; S2X259.1-LS-GA-afuc; S2X259-GAALIE; S2X259.1-LS; S2X259.1-LS-GA; S2X259.1-LS-GALVQE; S2X259.1-LS-GRLR. In FIG. 27H at the 10⁴ ng/mL antibody concentration, the curves are, from top to bottom: S2X259.1-LS-GARPYL; S2X259.1-LS-GALVQE-afuc; S2X259.1-LS-GA-afuc; S2X259.1-LS; S2X259-GAALIE; S2X259.1-LS-GA; S2X259.1-LS-GRLR; S2X259.1-LS-GALVQE. In FIG. 271 at the 10⁴ ng/mL antibody concentration, the top curve corresponds to S309-DEA and the bottom curve corresponds to S2X259.1-LS-GRLR. In FIG. 27J at the 10⁴ ng/mL antibody concentration, the top curve corresponds to S2X259-GAALIE and the bottom curve corresponds to S2X259.1-LS-GRLR.

FIGS. 28A-28D relate to certain anti-SARS-CoV-2 antibodies comprising variant Fcs. (A)-(B) FcγRIIIA activation/signaling as determined by luminescence using reporter cells expressing human FcγRIIIA driving luciferase expression (Promega) and, as target cells, ExpiCHO transfected with SARS-CoV-2 spike protein. (A) Wuhan-Hu-1 spike protein; (B) Mutation-stabilized Wuhan-Hu-1 spike protein, in which shedding of S protein from the target cell is not possible. (C)-(D) NK cell-mediated antibody-dependent cellular cytotoxicity (ADCC). Donor PBMC/primary NK cells expressing FcγRIIIA F158/V158 (C) or V158/V158 (D). Some Fc variants did not titrate as the signal is around the maximum/plateau of the assay.

FIGS. 29A-29Q relate to certain anti-HBV (“HBC34-v40”) Fc variant antibodies. (A)-(B) CD83+ cells in ex vivo HBV+ patient serum (as assessed by flow cytometry) with Fc-variant HBC34-v40 antibody and HBsAg, as indicated. In (B), each condition (30 HBsAg IU/mL, 100 HBsAg IU/mL, 300 HBsAg IU/mL, 1000 HBsAg IU/mL) shows four clusters of vertically dispersed data points. Under each HBsAg concentration condition: the left-most cluster corresponds to HBC34-v40-rIgG1-GRLR; the second-left cluster corresponds to HBC34-v40-rIgG1-LS; the second-right cluster corresponds to HBC34-v40-rIgG1-LS-GAALIE; and the right-most cluster corresponds to HBC34-v40-rIgG1-LS-GA. (C) Schematic showing design of MSD MULTI-SPOT® 96-Well 10-Spot Plate for measuring cytokine production. (D) Cytokine production by donor monocyte-derived dendritic cells (moDCs, 3 donors) against HBV+ serum (5 donors) and the indicated Fc variant antibody. Vertically dispersed data points are clustered as follows at each condition of HBsAg concentration: left-most cluster=HBC34-v40-GRLR; second-left cluster=HBC34-v40-rIgG1-LS; second-right cluster=HBC34-v40-rIgG1-LS-GAALIE; right cluster=HBC34-v40rIgG1-LS-GA. (E) Flow cytometry showing CD83 expression on moDCs (expressing the indicated FcγR) in the presence of the indicated HBC34-v40 Fc variant antibody (50 pg/mL) and 30 IU/mL HBsAg from HBV+ patient serum. (F) Flow cytometry showing CD83 expression on moDCs in the presence of HBC34-v40 Fc variant antibody (50 pg/mL) and HBsAg from HBV+ patient serum (BioIVT) at the indicated concentration. Left graphs are from an experiment using a first method of pipetting/generating immune complexes of antibody:HBsAg; right graphs are from an experiment using a second method of pipetting/generating immune complexes of antibody:HBsAg. (G) CD25 expression (marker of activation) and CFSE (proliferation) on autologous CD4+ memory T cells (from an HBV vaccinee) incubated for 5 days with moDCs from the same donor; moDCs were first activated overnight with with 100 IU/mL HBsAg (from two patient sera) and 50 μg/mL HBC34-v40 Fc variant antibody. The LS-GAYL variant was compared in one experiment. (H) % of CFSE low CD25+ human CD4+ memory T cells from HBV vaccines with the indicated Fc variant antibody and HBV+ patient sera. Vertically dispersed data points are clustered as follows in the left-most graph: left-most cluster=HBC34-v40-GRLR; second-left cluster=HBC34-v40-rIgG1-LS; second-right cluster=HBC34-v40-rIgG1-LS-GAALIE; right cluster ═HBC34-v40rIgG1-LS-GA. The clustering is the same in the center and right-hand graphs, with the addition of HBC34-v40-rIgGlm3-LS-GAYL as the right-most cluster. (I)-(J) CD14+ monocytes were stimulated with IL-4 and GM-CSF for 6 days. MoDCs were treated with antigen and HBC34-v40 Fc variant antibody (50 pg/mL) overnight, then co-cultured with an HLA-matched (HLA-DR-restricted) transgenic Jurkat cells expressing an HBsAg-specific human TCR. The readout was GFP-NFAT reporter of Jurkat cells. (K) Comparison of Jurkat TCR reporter assay for three independent experimental repeats at 0.125 μg/mL antibody. (L) Summary of data from different assays. (M) Scheme showing experimental setup for assessing ex vivo proliferation of T cells from FcγR-expressing mice immunized and boosted with an HBsAg vaccine; memory CD44+CD4+ T cells were sorted, labeled with CFSE, co-cultured with immune complex (antibody:HBsAg antigen)-pulsed BMDCs, and assessed for proliferation on day 6. SEB=Staphylococcal enterotoxin B from S. aureus/(N) CD4 expression and CFSE staining on (500,000) CD4+ memory T cells as in (M), wherein the BMDCs (50,000) were stimulated using immune complexes comprising the indicated HBC34-v40 Fc variant antibody (20 μg/mL) and HBsAg (1000 IU/mL). SEB=1 μg/mL; Mann-Whitney test. (N) (Left) Frequency of CFSE low CD4+CD44+ T cells following incubation with moDCs pre-treated with HBsAg alone, antibody alone, or SEB; (Right) Frequency of CFSE low CD4+CD44+ T cells following incubation with moDCs pre-treated with HBsAg and the indicated HBC34-v40 Fc variant antibody at the indicated concentration. moDCs were from mice transgenically expressing human FcγRs, and T cells were from either HuFcγR mice (n=4 independent experiments) or C57Bl/6 mice (n=1 experiment). 50,000 moDCs+500,000 T cells were tested. SEB=1 μg/mL; Mann-Whitney test. (P) left, schematic showing set-up for SPR assay to study binding of HBC34-v40 Fc variants to FcγR (CAP chip was used for capture of biotinylated FcγR proteins by Streptavidin; HBC34-v40-rIgGlm3 Fc variants were injected at concentrations of 819, 273, 91, 30.3, and 10.1 nM; injections were performed successively with no regeneration between different concentrations of the same sample; injection: 600 seconds; dissociation 100 seconds each injection), right example SPR curves showing binding to FcγRIIIA. (Q) Fold-change results for Fc variant antibodies, calculated by dividing the respective value for the control (HBC34-v40 rIgGlm3-LS) by the value determined for each variant. Binding was measured by a meso scale discovery-based assay (MSD; employing electrochemiluminescence)). A larger number reflects a decrease in KD and an increase in binding affinity. “-”=no binding.

### DETAILED DESCRIPTION

Provided herein are engineered polypeptides (e.g., Fc polypeptides, Fc polypeptide fragments, Fc fusion proteins, antibodies, and the like) that comprise a variant of an IgG Fc polypeptide (or a portion or fragment thereof), which variants (and the polypeptides that comprise these variants) have one or more improved characteristics over known Fc polypeptides (such as, for example, a reference wild-type Fc polypeptide and/or to a known variant Fc polypeptide) or polypeptides that comprise a known Fc polypeptide. Presently disclosed polypeptides possess, for example: increased binding to one or more human FcγRA (e.g., a FcγRIIA and/or a FcγRIIIA; decreased/reduced binding to a human FcγRIIB; increased binding to one or more human FcγRA as compared to binding to a human FcγRIIB; increased thermostability as compared to known Fc polypeptides; increased binding to human C1q; increased human FcγRIIIA signaling in a host cell expressing the FcγRIIIA, increased human FcγRIIIA signaling in a host cell expressing the FcγRIIA, decreased human FcγRIIB signaling in a host cell expressing the FcγRIIB, a relative increase in binding to FcγRA as compared to FcγRIIB, improved qualities for production as compared to known Fc polypeptides; and combinations of such features.

In certain embodiments, antibodies comprising a variant Fc polypeptide of the present disclosure provide surprising advantages, such as any one or more of the following: increased binding affinity (e.g. as determined by surface plasmon resonance, e.g. using a Biacore instrument and/or as determined by a electrochemiluminescence assay, such as a meso scale discovery (MSD) assay) for and/or inducing increased signaling (e.g. as determined using (1) an Fc variant antibody (2) antigen-expressing target cells and (3) reporter cells expressing one or more human FcγRA, optionally driving expression of a reporter gene such as, for example, GFP or luciferase) by one or more human FcγRA, as compared to the antibody comprising a reference Fc polypeptide not comprising the mutation(s) and/or fucosylation state; decreased binding affinity for and/or inducing decreased signaling of human FcγRIIB, as compared to the antibody comprising a reference Fc polypeptide not comprising the mutation(s) and/or fucosylation state; a unique and optionally improved binding profile across human FcγRIIA-H, human FcγRIIA-R, human FcγRIIB, human FcγRIIIA-F, and human FcγRIIIA-V, wherein improved binding comprises an overall increase in binding to and/or activation of FcγRA signaling relative to binding to and/or activation of inhibitory FcγR signaling, as compared to the antibody comprising a reference Fc polypeptide not comprising the mutation(s) and/or fucosylation state; increased binding affinity for human C1q, as compared to the antibody comprising a reference Fc polypeptide not comprising the mutation(s) and/or fucosylation state; no detrimental effect or no substantial detrimental effect on thermal stability, a reduced negative effect on thermal stability as compared to a variant Fc polypeptide or fragment thereof not comprising the mutation(s) and/or fucosylation state (e.g., a human IgG1 Fc comprising the mutations G236A, A330L, and I332E (e.g. having a smaller decreasing effect, or no decreasing effect, on melting temperature as compared to the antibody comprising a human IgG1 Fc comprising the mutations G236A, A330L, and I332E), or having a higher melting temperature than the antibody comprising a human IgG1 Fc comprising the mutations G236A, A330L, and I332E)); increasing specific lysis (e.g. via ADCC) by natural killer cells and/or PBMCs (e.g. expressing F158/V158 or V158/V158 FcγRIIIA) against antigen-expressing target cells, as compared to the antibody comprising a reference Fc polypeptide not comprising the mutation(s) and/or fucosylation state (e.g. the antibody comprising a human IgG1 Fc comprising the mutations G236A, A330L, and I332E); increasing ADCP by monocytes (e.g. CD14+ monocytes, optionally expressing F158/V158 FcγRIIA and R131/H131 FcγRIIA or F158/F158 FcγRIIA and R131/H131 FcγRIIA) against antigen-expressing target cells, as compared to the antibody comprising a reference Fc polypeptide not comprising the mutation(s) and/or fucosylation state; increasing the percentage of CD83+ cells (e.g. moDCs) and/or increasing expression of CD83 by moDCs in a sample when provided in combination with the antigen, as compared to the antibody comprising a reference Fc polypeptide not comprising the mutation(s) and/or fucosylation state, when provided in combination with the antigen; increasing production of one or more cytokine (optionally selected from the group consisting of IL-1β, IFN-γ, IL-6, and TNF-α) by moDCs in a sample when provided in combination with the antigen, as compared to the antibody comprising a reference Fc polypeptide not comprising the mutation(s) and/or fucosylation state, when provided in combination with the antigen; and/or increasing the ability of moDCs to stimulate antigen-specific CD4+ T cells when provided to the moDCs in combination with the antigen, as compared to the antibody comprising a reference Fc polypeptide not comprising the mutation(s) and/or fucosylation state, when provided to the moDCs in combination with the antigen, wherein, optionally, (1) the moDCs and the CD4+ T cells are from the same (optionally antigen-vaccinated) subject and/or (2) stimulation of antigen-specific CD4+ T cells is determined by an increase in CD25 expression and/or an increase in proliferation (e.g. as determined by a reduction in CFSE staining over time) and/or an increase in expression of CD69 and/or an increase in expression of NFAT and/or an increase in expression of CD44, by the antigen-specific CD4+ T cells.

In some embodiments, an engineered Fc or Fc fragment of the present disclosure (or a polypeptide comprising the same) comprises two or more substitution mutations as compared to a reference wild-type Fc or Fc fragment, and the combined effect of the two or more substitutions is different than, and is optionally greater than, would be expected based on the effects of the individual component substitution mutations and/or based on the effects of a subset of the two or more substitution mutations. In other words, in some embodiments, combination mutations comprise a non-additive or synergistic effect with reference to the individual component mutations and/or to a subset thereof.

In some embodiments, presently disclosed variants and polypeptides comprising the same possess characteristics such as effector functions, ability to bind human C1q, ability to induce FcγRA-mediated cell signaling, ability to bind to human FcRn, ability to promote ADCP, ability to promote ADCC, ability to promote activation of CD4+ T cells, and the like. In some embodiments, an engineered polypeptide of the present disclosure comprises an antibody, an Fc fusion protein, or a conjugate comprising the same. Also provided are antibodies that comprise a variant IgG Fc according to the present disclosure.

In certain embodiments, presently disclosed polypeptides and antibodies have one or more altered characteristics (e.g., increased binding to a human FcγRa, decreased binding to a human FcγRIIb, binding to a human FcγRa that is increased relative to the binding to a FcγRIIb, increased binding to a human C1q, increased binding to a human FcRn, an increased Tm, increased binding to a FcγRIIIa, or any combination thereof), as compared to a reference polypeptide or antibody that comprises a variant Fc containing the following mutation(s): G236A; G236S; G236A/A330L/I332E; G236A/A330L/I332E/M428L/N434S; G236A/A330L/I332E/M428L/N434A; G236A/S239D/A330L/I332E; or A330L/I332E.

Also provided are related polynucleotides, vectors, host cells, and compositions.

The presently disclosed compositions and methods are useful, in various embodiments, to treat and/or prevent disease. In some embodiments, a presently disclosed composition can be administered in any stage of a disease (e.g., during an early stage of an infection, during a late stage of an infection, when an infection is established, or at any other timepoint during an infection) and can protect against and/or neutralize the infection, promote clearance of infected cells, block the spread of infection, stimulate host anti-infective adaptive immunity, or the like.

It will be understood that herein, “FcγRIIA” may be expressed as “FcγRIIa”, “FcγRIIIA” may be expressed as “FcγRIIIa”, “FcγRIIB” may be expressed as “FcγRIIb”, and “FcγRIIIB” may be expressed as “FcγRIIIb”.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. For example, in some embodiments, the term “about” can refer to ±15%, ±10%, or ±5% of the indicated range, value, or structure. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, a Fc, a CH2, a CH3, a CH2-CH3, or a CH1-CH3) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

In certain embodiments, a variant of a CH2, CH3, CH1-CH3, or Fc polypeptide comprises one or more amino acid substitutions relative to a wild-type or parent CH2, CH3, CH1-CH3, or Fc polypeptide, respectively, wherein the one or more amino acid substitutions comprise, consist essentially of, or consist of the specifically recited amino acid substitution(s). In some embodiments, a variant of a CH2, CH3, CH1-CH3, or Fc polypeptide comprises only the specifically recited substitution mutation(s) relative to a wild-type or parent CH2, CH3, CH1-CH3, or Fc polypeptide, respectively. In other embodiments, a variant of a CH2, CH3, CH1-CH3, or Fc polypeptide comprises the specifically recited substitution mutation(s) and one or more additional amino acid substitution mutation(s) (e.g. in some embodiments, one or more conservative amino acid substitution and/or one or more amino acid substitution mutation(s) that are physically remote in tertiary structure of the Fc polypeptide or fragment thereof from the specifically recited one or more amino acid substitution mutation(s)), provided that one or more characteristics of the claimed subject-matter is retained or substantially retained and is not materially changed, e.g. binding to and/or activation of one or more FcγR, binding to FcRn, melting temperature, binding to C1q, promotion of ADCC, promotion of ADCP, promotion of CDC, formation of an immune complex, activation of dendritic cells (e.g. monocyte-derived dendritic cells) when provided in an immune complex with antigen, or the like. In some embodiments, a claimed subject-matter comprising one or more amino acid substitution(s) that consist(s) essentially of the specified amino acid substitution(s) is a functional variant of a claimed subject-matter wherein the amino acid substitution(s) consist(s) of the specified amino acid substitution(s).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). Examples of substitution mutations in Fc polypeptides and Fc sequences comprising these are shown in Table 1 and in the Sequence Listing.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

In some embodiments, the polynucleotide (e.g. mRNA) comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine. These features are known in the art and are discussed in, for example, Zhang et al. Front. Immunol., DOI=10.3389/fimmu.2019.00594 (2019); Eyler et al. PNAS 116(46): 23068-23071; DOI: 10.1073/pnas.1821754116 (2019); Nance and Meier, ACS Cent. Sci. 2021, 7, 5, 748-756; doi.org/10.1021/acscentsci.1c00197 (2021), and van Hoecke and Roose, J. Translational Med 17:54 (2019); https://doi.org/10.I186/si2967-019-1804-8, which modified nucleosides and mRNA features are incorporated herein by reference.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. “Isolated” can, in some embodiments, also describe an antibody, antigen-binding fragment, polypeptide, polynucleotide, vector, host cell, or composition that is outside of a human body.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, amino acid sequence, compound, nucleic acid molecule, or activity that is not native to a host cell or a subject, or any gene, protein, amino acid sequence, compound, nucleic acid molecule, or activity native to a host cell or a subject that has been altered. Heterologous, non-endogenous, or exogenous includes genes, proteins, amino acid sequences, compounds, or nucleic acid molecules that have been mutated or otherwise altered such that the structure, activity, or both is different as between the native and altered genes, proteins, amino acid sequences, compounds, or nucleic acid molecules. In certain embodiments, heterologous, non-endogenous, or exogenous genes, proteins, amino acid sequences, or nucleic acid molecules (e.g., receptors, ligands, etc.) may not be endogenous to a host cell or a subject, but instead nucleic acids encoding such genes, proteins, amino acid sequences, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, amino acid sequence, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mites et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure). A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In the context of a disease, “host” can refer to a cell or a subject having the disease. For example, as discussed further herein, a variant of an Fc polypeptide can be administered to improve or modulate a host immune response against a pathogen or the like that is afflicting the host.

“Antigen” or “Ag”, as used herein, refers to an immunogenic molecule that provokes an immune response. This immune response may involve antibody production, activation of specific immunologically competent cells, fixation of complement, antibody-dependent cell-mediated cytotoxicicity (also called antibody-dependent cellular cytotoxicity), antibody-dependent cellular phagocytosis, production of cytokines, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in a betacoronavirus (e.g., a surface glycoprotein or portion thereof), such as present in a virion, or expressed or presented on the surface of a cell infected by a betacoronavirus.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

Fc Polypeptide Variants and Polypeptides that Comprise the Same

The present disclosure provides, in part, engineered variants of immunoglobulin G (IgG) Fc polypeptides and fragments or portions thereof, and proteins (e.g. antibodies and fusion proteins) that comprise these. By way of background, the Fc region (also called the “Fc domain”) of an antibody can interact with Fc receptors and other binding partners such a complement C1q to, for example, initiate, participate, and/or and mediate an immune response against a pathogen or antigen. Presently disclosed Fc variants possess various advantages over native (i.e. wild-type) Fc and/or known Fc variants, such as, but not limited to, increased binding to one or more activation or activating Fc receptor (e.g. FcγRIIa), decreased binding to an inhibitory Fc receptor (e.g. FcγRIIb), providing a relative increase in binding to an activating Fc receptor versus to an inhibitory Fc receptor, binding to complement C1q, facilitating or increasing antibody-dependent cellular phagocytosis (ADCP), facilitating or increasing antibody-dependent cell cytotoxicity (ADCC), facilitating or increasing complement, facilitating or increasing intracellular signaling that occurs via an activating Fc receptor, reducing intracellular signaling that occurs via an inhibitory Fc receptor, providing a relative increase in signaling via an activating Fc receptor versus signaling via an inhibitory Fc receptor, facilitating or increasing activation of dendritic cells (e.g. monocyte-derived dendritic cells) when provided thereto in a (variant Fc-bearing) antibody:antigen complex, or the like. In certain embodiments, presently disclosed Fc variants possess improved thermal stability (e.g., a higher Tm, or a Tm that is closer to the Tm of a wild-type Fc polypeptide), similar or improved capacity for production and/or purification, and/or favorable binding to FcRn, e.g. as compared to a wild-type reference Fc polypeptide or a variant Fc polypeptide that does not comprise the specified mutation(s) and/or fucosylation state.

As discussed further herein, immunoglobulins typically include two heavy chain polypeptides. An immunoglobulin heavy chain typically includes a variable region (also called a variable domain) and a constant region (also called a constant domain). In that case of, for example, the IgG isotype, the constant region typically comprises a CH1 region, a hinge, CH2, and CH3. Heavy chain polypeptide monomers can associate and be held together by shared disulfide bonds to form a dimer; the CH2-CH3 portions of an immunoglobulin heavy chain dimer comprise the Fc (fragment crystallizable) portion or domain of an immunoglobulin, for example, an IgG1 antibody. An example of a wild-type human IgG1 CH1-CH3 amino acid sequence is provided in SEQ ID NO.: 1. An example of a wild-type human IgG1 hinge-CH2-CH3 is provided in SEQ ID NO.:2. An example of a wild-type human IgG1 CH2 is provided in SEQ ID NO.: 3. An example of a wild-type human IgG1 CH3 amino acid sequence is provided in SEQ ID NO.:4. An example of a wild-type human IgG1 hinge-CH2 amino acid sequence is provided in SEQ ID NO.:5. It will be understood that a hinge of a hinge-CH2 polypeptide or a hinge-Fc polypeptide can comprise one or more modifications (e.g. mutations) relative to a wild-type hinge sequence, which one or more modification can be further to, e.g., a P230A or S219Y mutation as disclosed herein.

As used herein, unless the context provides otherwise, an “Fc polypeptide” refers to a CH2-CH3 polypeptide. A fragment of an Fc polypeptide can comprise a CH2, a portion of a CH2, a CH3, and/or a portion of a CH3, while not comprising a complete, full-length CH2-CH3. In certain embodiments, Fc polypeptide fragments are provided that comprise a portion of a CH2 and/or a CH3 of sufficient length to comprise the specified amino acid position(s) and variations and, in some embodiments, to possess the recited function or functions.

Presently disclosed polypeptides include those that comprise a variant of: an IgG Fc polypeptide or a fragment thereof, wherein the variant comprises one or more modifications as compared to the IgG Fc polypeptide or fragment thereof. It will be understood that, unless stated otherwise, a “reference” polypeptide or antibody (e.g., reference IgG Fc polypeptide or fragment thereof, reference antibody, reference CH2 polypeptide, reference IgG hinge-CH2, reference IgG hinge-Fc polypeptide, reference CH3 polypeptide) is preferably identical to the recited molecule (e.g., variant of an Fc polypeptide or fragment thereof; polypeptide comprising such a variant; antibody comprising a variant of an Fc polypeptide) except for the recited difference or differences.

For example, it will be understood that for a variant IgG1 Fc polypeptide that comprises an alanine (A) amino acid at EU position 236, a reference Fc polypeptide includes an IgG1 Fc polypeptide that is otherwise identical to the variant except that a native glycine (G) amino acid is found at EU position 236. As another example, for a variant of an Fc polypeptide fragment (e.g., containing a CH2 and a portion of a CH3), a reference Fc polypeptide fragment is preferably of an identical length to the variant and preferably differs from the variant only by the recited features (e.g., amino acid mutation or mutations present in the variant). In some embodiments, a reference Fc polypeptide, Fc polypeptide fragment, or antibody comprises a wild-type amino acid sequence (e.g., wild-type human IgG1). Excepting the recited differences present in the variant, a reference Fc polypeptide, Fc polypeptide fragment, or antibody will be of the same isotype, and, preferably, of the same allotype, as the variant. In the case of a reference antibody, the Fabs or other antigen-binding domains will preferably be identical to those present in the specified antibody comprising a variant Fc polypeptide or fragment thereof.

In some embodiments, variants of IgG Fc polypeptides or fragments thereof include one or more amino acid substitution as compared to a reference (e.g. wild-type) IgG Fc polypeptide or fragment thereof. Herein, the position of an amino acid in a variant IgG Fc polypeptide or fragment may be described by referencing the “EU position”; it will be understood that “the EU position” follows the EU numbering system as set forth in Kabat. By way of illustration, it will be understood that in the example of a human IgG1 CH1-CH3 amino acid sequence provided in SEQ ID NO.:1, the first amino acid (A) corresponds to EU position 118, and the last amino acid (K) corresponds to EU position 447:

(SEQ ID NO.: 1)
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLSPGK

Accordingly, it will be understood that unless otherwise indicated, the position of a recited amino acid(s) follows EU numbering for human IgG1 even if a complete antibody heavy chain, complete CH1-CH3, complete CH2-CH3, or the like is not present or is not explicitly recited. In other words, for example, if only a hinge-CH2 is described and a CH3 and/or CH1 may not be present, the position of the amino acids in the hinge-CH2 is described with reference to EU numbering, unless stated otherwise. Correspondence between EU numbering, Kabat numbering, IMGT exon numbering, and IMGT unique numbering for immunoglobulin G heavy chain constant domain is known in the art and is shown, for example, in the IMGT Scientific chart (www.imgt.org/IMGTScientificChart/Numbering/Hu_IGHGnber.html; created May 17, 2001, accessed May 23, 2021, last updated Jan. 20, 2020).

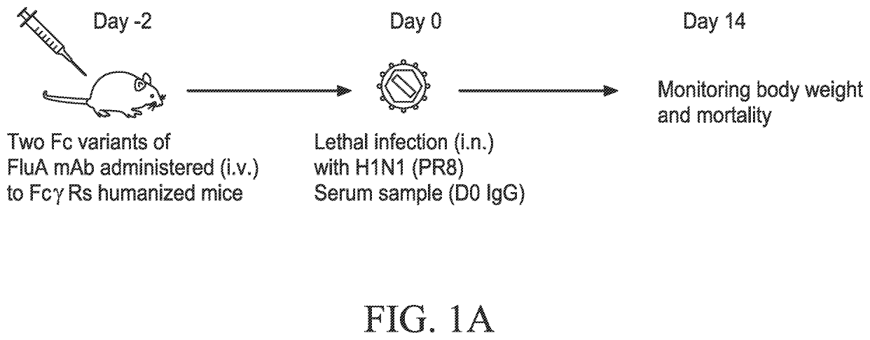

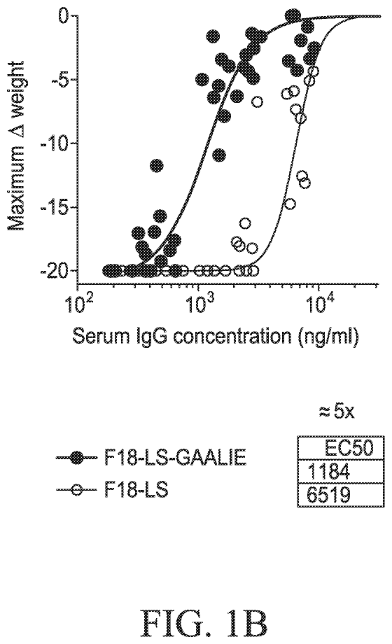

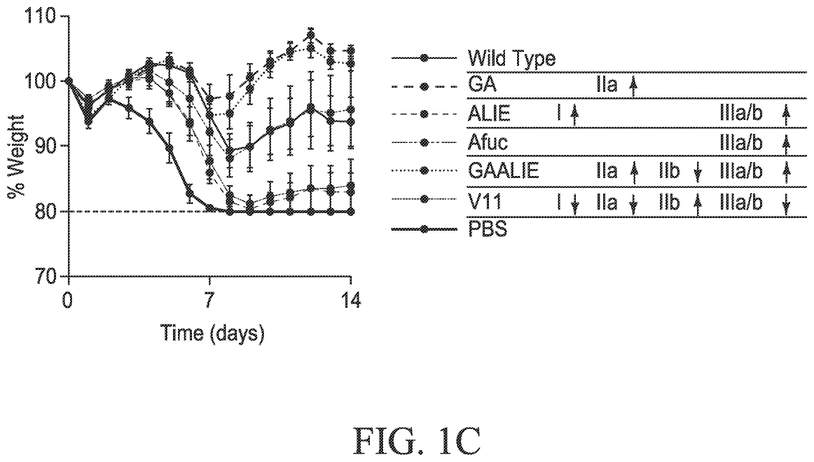

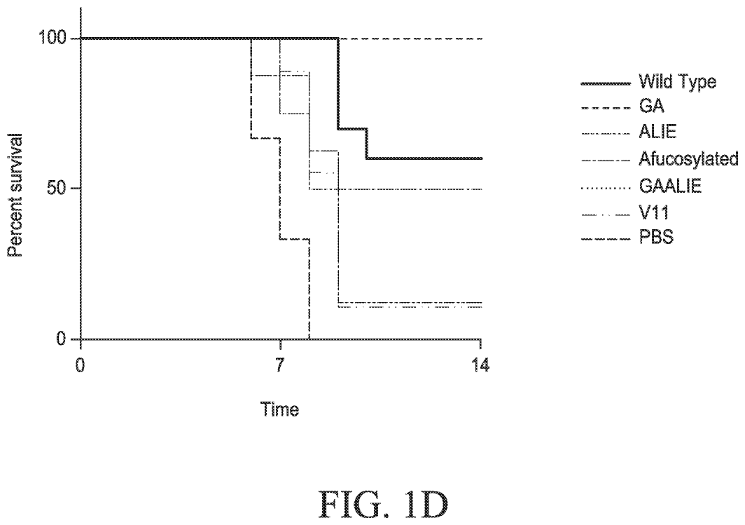

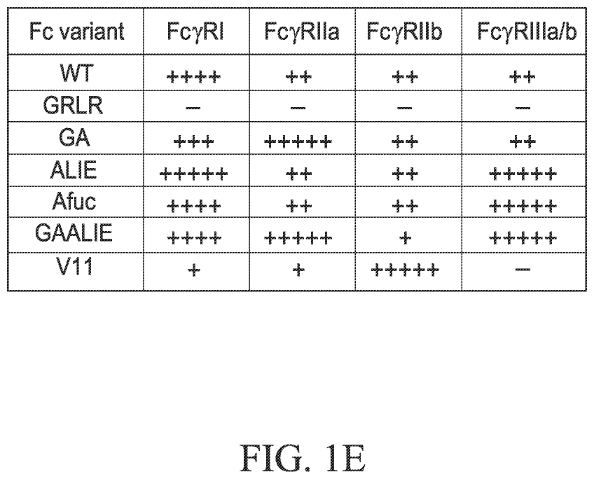

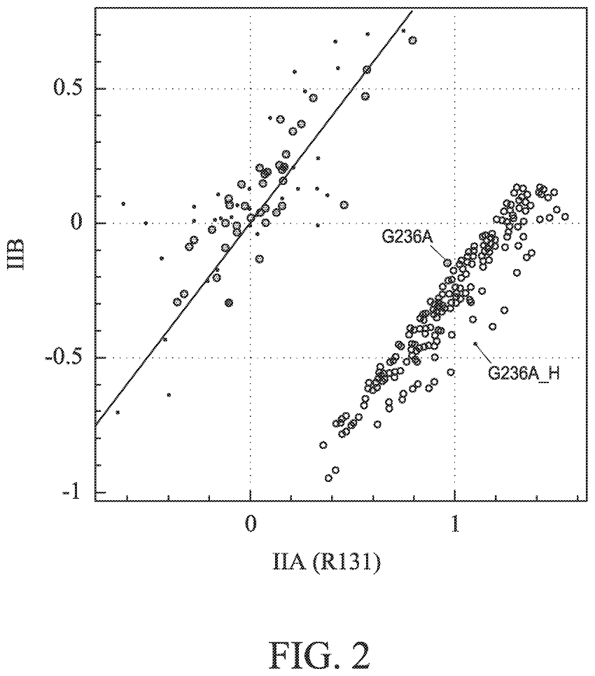

In the present Examples, certain Fc variants were generated (expressed in fucosylated and afucosyated human IgG1 antibodies of various allotypes) and tested for various properties. Certain embodiments of Fc variants of the present disclosure (fucosylated, unless otherwise indicated) and non-limiting properties of the same are summarized in Table 1; see also FIGS. 10A-1-10C.

TABLE 1
Certain Fc variants and properties thereof

| Variant | |
|---|---|
| (substitution mutation(s) vs. | Certain properties of the indicated variant(s), as |
| wild-type human IgG1 Fc) | compared to fucosylated wild-type human IgG1 |
| G236A_L328V_Q295E | Increased binding to human FcγRIIa (H131 allele and |
| G236A_P230A_Q295E | R131 allele); comparable or decreased binding to human |
| G236A_R292P_I377N | FcγRIIb (e.g. by MSD assay and/or surface plasmon |
| G236A_K334A_Q295E | resonance); increased ratio of: binding to human FcγRIIa |
| G236S_R292P_Y300L | (H131 allele or R131 allele) versus binding to human |
| | FcγRIIb; comparable binding to human FcRn; |
| | comparable production titer; increased signaling in a host |
| | cell via FcγRIIa and/or decreased signaling in a host cell |
| | via FcγRIIb; Tm within 12° C. or less of wild-type; |
| | G236S_R292P_Y300L has improved binding to C1q |
| G236A_Y300L | Increased binding to human FcγRIIa (H131 (over 18- |
| | fold) and R131 (over 4-fold)); similar binding to human |
| | FcγRIIb or reduced binding to human FcγRIIb (e.g. as |
| | measured by surface plasmon resonance); increased ratio |
| | of: binding to human FcγRIIa (H131 or R131) versus |
| | binding to human FcγRIIb; comparable binding to |
| | human FcRn; comparable production titer; increased |
| | signaling in a host cell via FcγRIIa and/or decreased |
| | signaling in a host cell via FcγRIIb; Tm within 4.5° C. of |
| | wild-type |
| G236A_R292P_Y300L | Increased binding to human FcγRIIa (H131 (over 14- |
| | fold) and R131 (over 2.7-fold)); similar binding to |
| | human FcγRIIb; increased ratio of: binding to human |
| | FcγRIIa (H131 or R131) versus binding to human |
| | FcγRIIb; increased binding to human FcγRIIIa (V158 |
| | allele and F158 allele); comparable binding to human |
| | FcRn; comparable production titer; increased signaling |
| | in a host cell via FcγRIIa and/or FcγRIIIa, and/or |
| | decreased signaling in a host cell via FcγRIIb; increased |
| | signaling in a host cell via FcγRIIa and/or decreased |
| | signaling in a host cell via FcγRIIb; Tm within 4° C. of |
| | wild-type; comparable binding to human C1q |
| G236S_G420V_G446E_L309T | Increased binding to human FcγRIIa; decreased binding |
| G236A_R292P | to human FcγRIIb (less than 0.5-fold); increased ratio of: |
| | binding to human FcγRIIa (H131 or R131) versus |
| | binding to human FcγRIIb; comparable binding to |
| | human FcRn; comparable production titer; increased |
| | signaling in a host cell via FcγRIIa and/or FcγRIIIa, |
| | and/or decreased signaling in a host cell via FcγRIIb; Tm |
| | within 4° C. or less of wild-type |
| R292P_Y300L | Increased binding to human FcγRIIIa (V158 and F158); |
| | increased binding to human C1q; Tm within 4° C. of wild- |
| | type |
| Y300L | Increased binding to human C1q |
| E345K_G236S_L235Y_S267E | |
| E272R_L309T_S219Y_S267E | |
| G236Y | |
| G236W | |
| F243L_G446E_P396L_S267E | |
| G236A (afucosylated) | Increased binding to human FcγRIIa (H131) and mouse |
| | FcγRIIa (R131), decreased binding human FcγRIIb, |
| | increased binding to human FcγRIIIa (V158) and mouse |
| | FcγRIIIa (F158), increased binding to human FcγRIIIb, |
| | somewhat decreased binding to human FcRn, Tm within |
| | 0.15° C. of wild-type or within 0.9° C. of wild-type or |
| | within 0.8° C. of wild-type or within 0.7° C. of wild-type |
| S239D_H268E_G236A | Increased binding to and signaling via all human FcγRs |
| | tested: FcγRIIA (H131); FcγRIIA (R131); FcγRIIB; |
| | FcγRIIIA (V158); FcγRIIIA (F158); FcγRIIIB; |
| | additionally, when anti-HBV antibody bearing |
| | S239D_H268E_G236A_M428L_N434S was combined |
| | with HBsAg, the immune complexes formed thereby |
| | were incubated with MoDCs; subsequent incubation of |
| | the MoDCs with donor CD4+ T cells resulted in an |
| | increased percentage of NFAT+ CD69+ CD3+ CD4+ T |
| | cells as compared to antibodies bearing M428L_N434S |
| | only. |

Additional features of disclosed Fc variant-containing antibodies are shown in the present Examples and Figures, and described herein. For example, FIGS. 10B-1-10-B-4 show certain properties of antibodies comprising certain afucosylated variant Fcs.

It will be understood that two or more amino acid substitutions present in a variant can be expressed in a variety of ways, for example, as G236A_Y300L, or as G236A/Y300L. Moreover, a mutation or combination mutation may be referenced using a short form including the original amino acid(s) and the amino acid(s) resulting from the substitution(s). For example, G236A may be described as “GA” or “236A”; G236A_Y300L may be described as “GAYL”; G236A_L328V_Q295E may be described as “GALVQE”; G236A_R292P_Y300L may be described as “GARPYL”, G236A_R292P_I377N may be described as “GARPIN”, or the like.

In any of the presently disclosed embodiments, a variant of an Fc polypeptide or fragment thereof can be derived from or comprise a human Fc polypeptide or fragment thereof, and/or can be derived from or comprise a human IgG1, a human IgG2, a human IgG3, or a human IgG4 isotype. In this context, the expression “derived from” means that the variant is the same as the referenced polypeptide or isotype, with the exception of the specified modification(s) (e.g., amino acid substitution(s)). By way of example, a variant Fc polypeptide which comprises a wild-type human IgG1 Fc amino acid sequence with the exception of the amino acid substitution mutations G236A_L328V_Q295E (and, optionally, other amino acid substitutions) can be said to be “derived from” wild-type human IgG1 Fc. In any of the presently disclosed embodiments, a polypeptide, CH2, Fc, Fc fragment, or antibody may comprise human Ig sequence, such as human IgG1 sequence. In some embodiments, the polypeptide, CH2, Fc, Fc fragment, or antibody can comprise a native or wild-type human Ig sequence with the exception of the described mutation(s), or can comprise a human Ig (e.g. IgG) sequence that contains one or more additional mutations.

In certain embodiments, a polypeptide comprises only the specified or recited amino acid mutations (e.g. substitutions), and does not comprise any further amino acid substitutions or mutations; e.g., relative to the reference polypeptide (e.g., a wild-type Fc polypeptide or fragment thereof). For example, in some embodiments, a variant Fc polypeptide comprising the amino acid substitutions G236A_Y300L does not comprise any other amino acid substitutions; i.e., comprises an amino acid sequence that is wild-type except for G236A and Y300L.

In some embodiments, a polypeptide may comprise one or more additional amino acid mutations (e.g. substitutions), which can be specified (e.g., M428L_N434S; M428L_N434A). In some embodiments, a further amino acid mutation or mutations is physically remote to the recited amino acid positions in tertiary structure, and/or is of such nature (e.g. is a conservative substitution), so that one or more function of the recited Fc variant or fragment thereof is not reduced or is reduced by no more than 50%, no more than 40%, no more than 30%, no more than 25%, no more than 20%, no more than 15%, no more than 10% or no more than 5%, or by no more than 10-fold, no more than 9-fold, no more than 8-fold, no more than 7-fold, no more than 6-fold, no more than 5-fold, no more than 4-fold, no more than 3-fold, no more than 2-fold, or no more than 1.5-fold. In some embodiments, a polypeptide comprises the mutations M428L and N434S or the mutations M428L and N434A, or any other mutation(s) that enhance binding to a human FcRn, including those described herein.

In some embodiments, a polypeptide is provided that comprises at least a portion of a(n e.g. human) IgG1 CH2-CH3 or hinge-CH2-CH3 or CH1-CH3 comprising the amino acid mutation(s) set forth in any one of (i)-(xviii): (i) G236A, L328V, and Q295E; (ii) G236A, P230A, and Q295E; (iii) G236A, R292P, and I377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E, wherein the numbering of amino acid residues is according to the EU index as set forth in Kabat. In certain embodiments, the polypeptide is afucosylated. In some embodiments, the IgG1 CH2-CH3 or hinge-CH3-CH3 or heavy chain has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a wild-type human IgG1 CH2-CH3 or hinge-CH2-CH3 or CH1-CH3, respectively. In certain embodiments, a polypeptide of the present disclosure comprises an Fc variant comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in any one of SEQ ID NOs.:1-5 and 36-38.

In some embodiments, an antibody (described further herein) is provided that comprises, in a(n e.g. human) IgG1 heavy chain, the amino acid mutation(s) set forth in any one of (i)-(xviii): (i) G236A, L328V, and Q295E; (ii) G236A, P230A, and Q295E; (iii) G236A, R292P, and I377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E; (xviii) G236A, S239D, and H268E, wherein the numbering of amino acid residues is according to the EU index as set forth in Kabat. In certain embodiments, the polypeptide is afucosylated. In some embodiments, the polypeptide or antibody further comprises one or more mutation that enhances binding to a human FcRn, such as M428L and N434S mutations or M428L and N434A mutations (EU numbering) or any other mutation(s) that enhance binding to a human FcRn, such as those described herein. In certain embodiments, the polypeptide is afucosylated. In some embodiments, the IgG1 heavy chain comprises a CH1-CH3 or a CH2-CH3 or a hinge-CH2-CH3, wherein the CH1-CH3 or CH2-CH3 or hinge-CH2-CH3 has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a wild-type human IgG1 CH1-CH3 or CH2-CH3 or hinge-CH2-CH3, respectively. In certain embodiments, an antibody of the present disclosure comprises an Fc variant comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in any one of SEQ ID NOs.:1-5 and 36-38.

In some embodiments, the polypeptide or antibody comprises the amino acid sequence set forth in any one of SEQ ID NOs.:6-23 and 45, or a variant thereof, e.g. that further comprises one or more mutation that enhances binding to a human FcRn, such as M428L and N434S mutations or M428L and N434A mutations (EU numbering) or any other mutation(s) that enhance binding to a human FcRn, including those described herein. In some embodiments, the polypeptide or antibody comprises an amino acid sequence that differs from the amino acid sequence set forth in any one of SEQ ID NOs.:6-23 and 45 only by one or more IgG1 allotype-specific mutations and/or by the presence of M428L and N434S mutations or M428L and N434A mutations or other mutation(s) that enhance binding to a human FcRn.

A polypeptide of the present disclosure can be fucosylated (e.g., comprising one or more fucosyl moiety, and typically comprising a native (wild-type) fucosylation pattern or a fucosylation pattern that includes one or more additional, or fewer, fucosyl moieties as compared to native), or can be afucosylated. In particular, native IgG1 antibodies carry a glycan site at N297, and this is typically the only site where a core fucose moiety may be found in the antibody, though some glycan sites may arise through mutation (e.g. in the variable domains) during antibody development. Fucosylation of an Fc polypeptide or fragment thereof, or of an antibody, can be effected by introducing amino acid mutations to introduce or disrupt a fucosylation site (e.g. a mutation at N297, such as N297Q or N297A, to disrupt formation of a glycan that can include a core fucose moiety), though typically it is preferred to maintain N297 and the glycan thereof, such as by expressing the polypeptide in a host cell which has been genetically engineered to lack the ability (or have an inhibited or compromised ability) to fucosylate the polypeptide; by expressing the polypeptide under conditions in which a host cell is impaired in its ability to fucosylate the polypeptide (e.g., in the presence of 2-fluoro-L-fucose (2FF)), or the like. An afucosylated polypeptide can comprise no fucose moieties, or substantially no fucose moieties, and/or can be expressed by a host cell that is genetically engineered to lack the ability (or have an inhibited or compromised ability) to fucosylate the polypeptide and/or can be expressed under conditions in which a host cell is impaired in its ability to fucosylate the polypeptide (e.g., in the presence of 2-fluoro-L-fucose (2FF)). In some embodiments, a polypeptide does not comprise a core fucose moiety at Asn297. In some embodiments, afucosylated polypeptides have increased binding to FcγRIIIA. In some contexts, addition of 2FF to a culture media comprising host cells expressing an antibody results in about 85% or more of the antibodies not carrying a fucose moiety. Accordingly, a plurality of antibodies may be described as “afucosylated” when the plurality was produced in the presence of 2FF or like reagent. In some contexts, a plurality of polypeptides or antibodies may be described as, for example, afucosylated, meaning that about 85% or more of the single polypeptide or antibody molecules of the plurality do not comprise a fucose moiety. In certain preferred embodiments, an afucosylated antibody or polypeptide or a population or a plurality thereof comprises an asparagine (N) at EU position 297. Fucosylation or lack thereof can be assessed using, for example, mass spectrometry (e.g. Electrospray mass spectrometry (ESI-MS)). In some embodiments, compositions are provided that comprise a plurality of any one or more of the presently disclosed polypeptides, wherein the composition comprises afucosylated polypeptides.

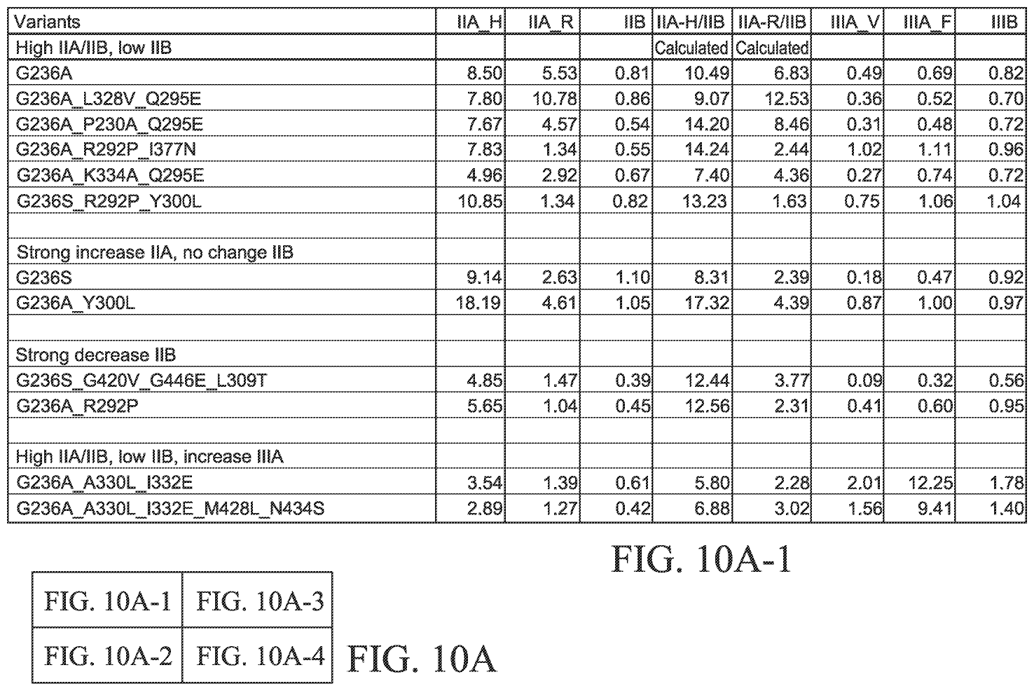

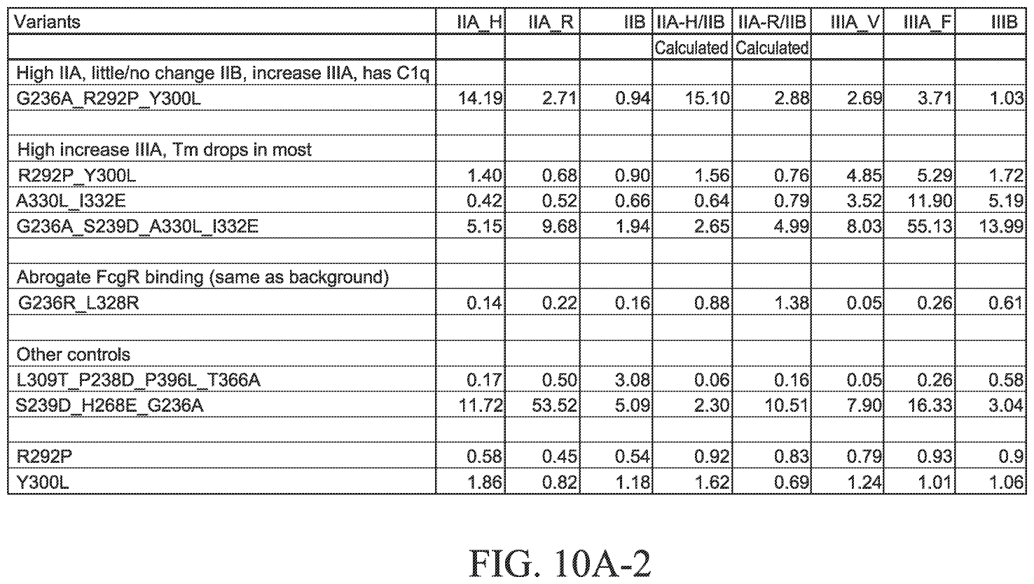

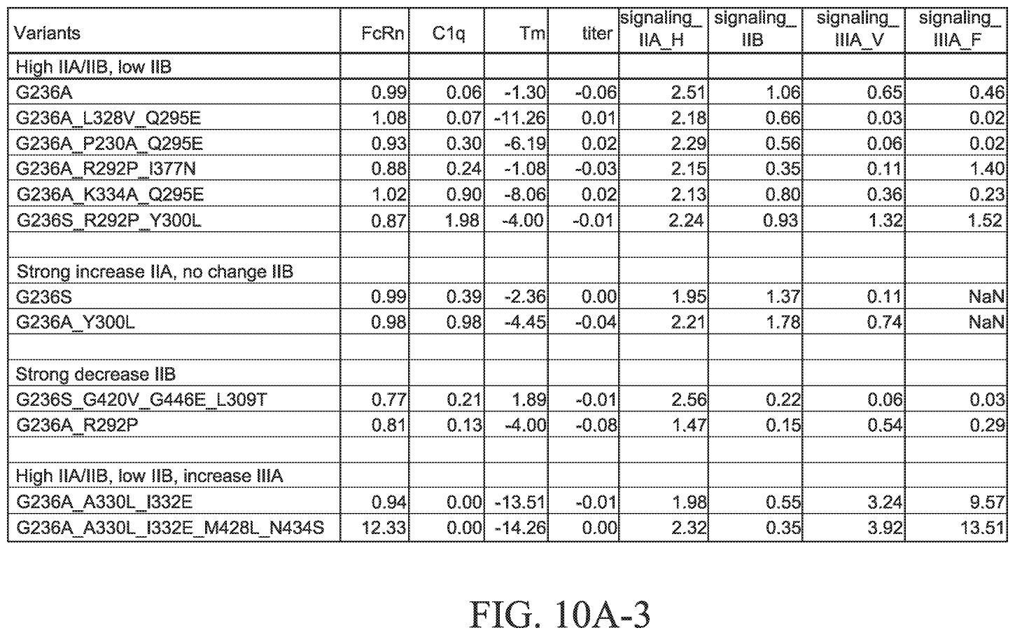

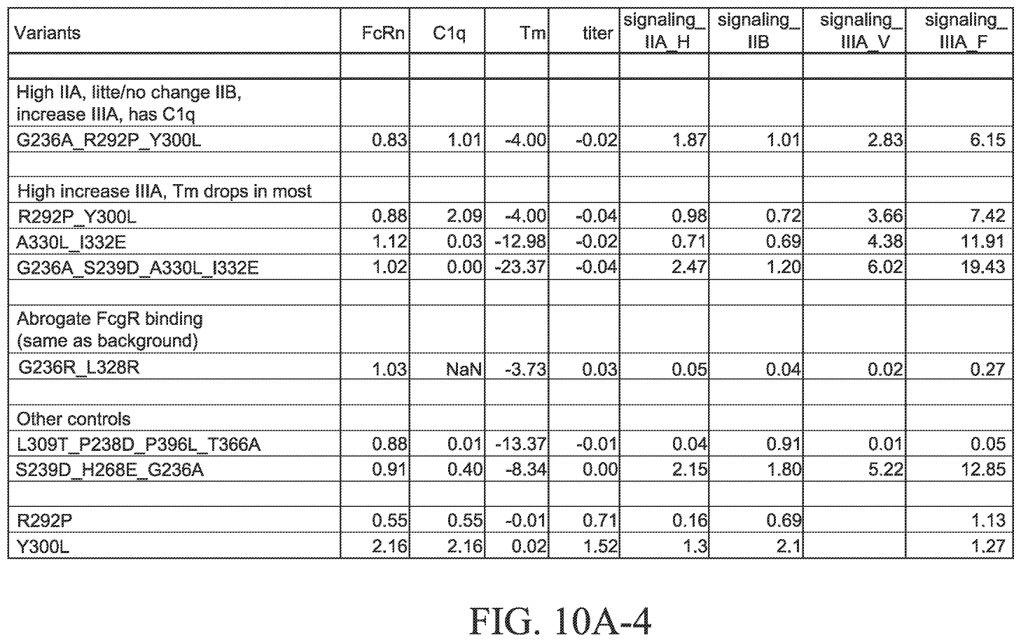

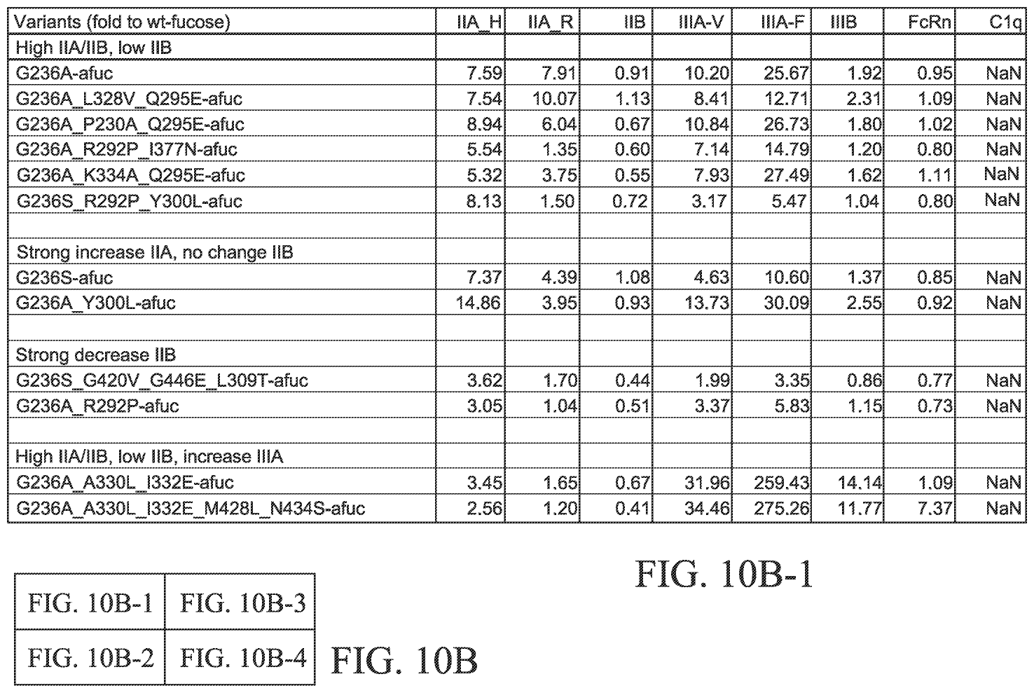

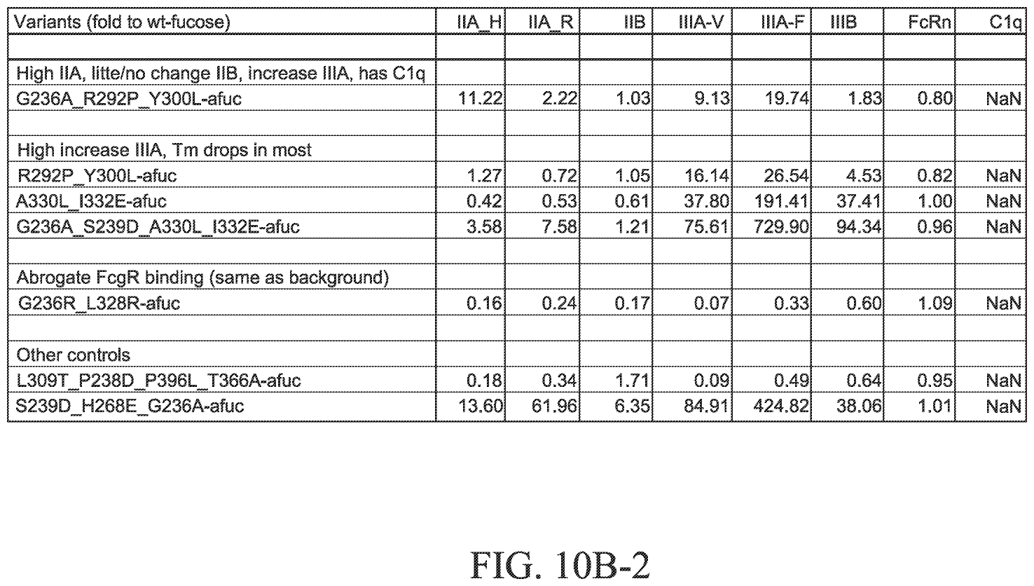

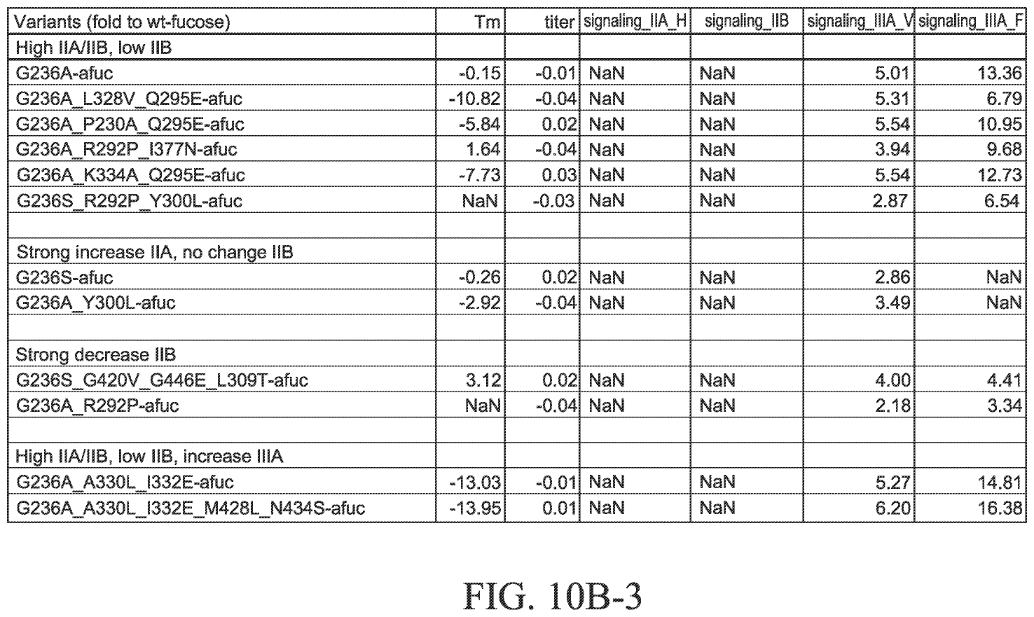

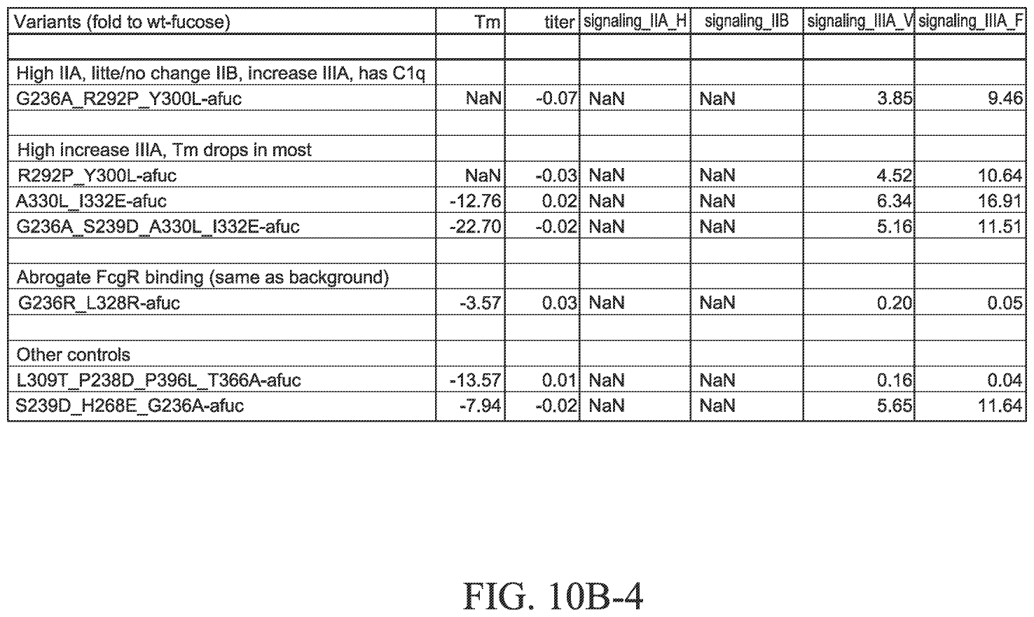

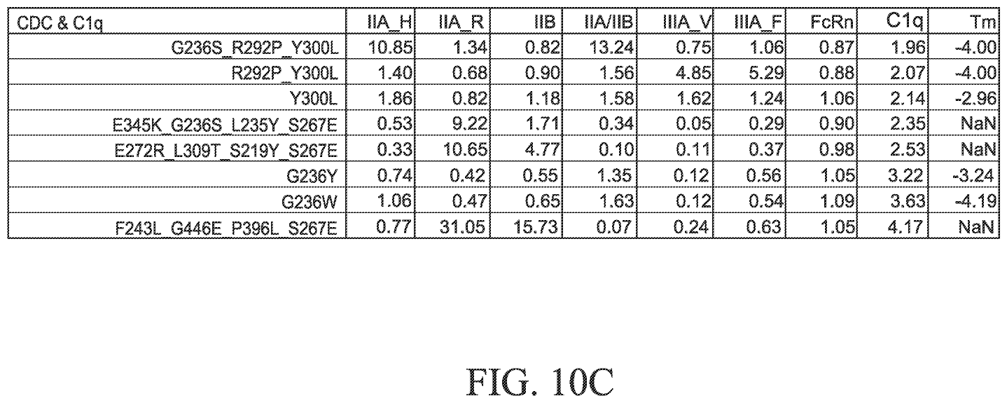

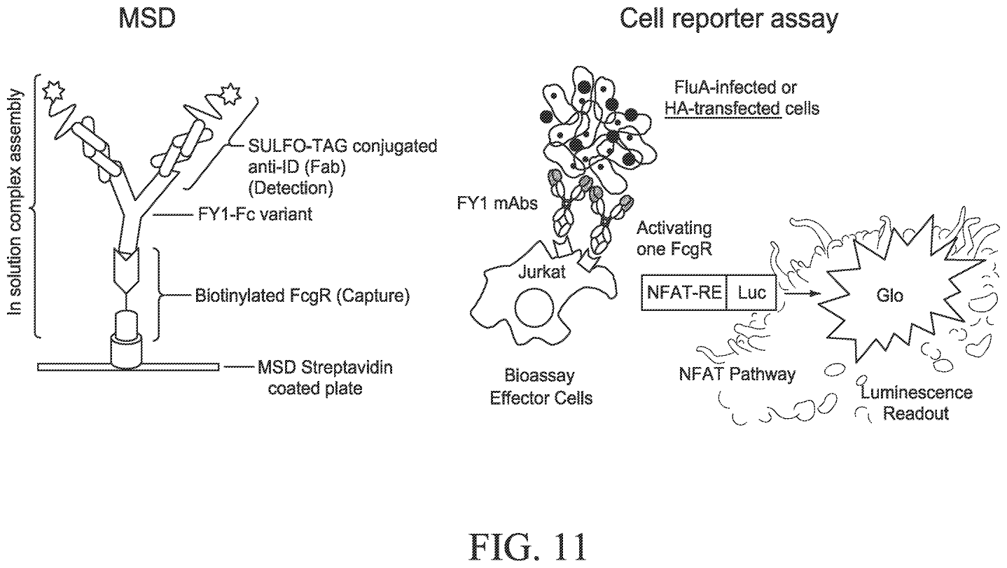

Also in the present Examples, variant Fcs including those comprising the mutations shown in Table 1 above were expressed in afucosylated human IgG1 antibodies and tested for various properties, including by comparison to fucosylated wild-type human IgG1 antibody. See e.g. FIG. 10B; in some contexts, afucosylated polypeptides bearing Fc variants have similar or even improved properties as when fucosylated.

In certain embodiments, presently disclosed variants of IgG Fc polypeptides or fragments thereof possess one or more function that is distinct from (e.g. improved as compared to) the corresponding function of a reference Fc polypeptide that comprises the following mutation or mutations: G236A; G236S; G236A_A330L_I332E; G236A_A330L_I332E_M428L_N434S; A330L_I332E; or G236A_S239D_A330L_I332E. For example, in certain embodiments, a presently disclosed variant of an IgG Fc polypeptide or fragment thereof possesses one or more of the following properties, as compared to a reference Fc polypeptide that comprises the following mutation or mutations: G236A; G236S; G236A_A330L_I332E; G236A_A330L_I332E_M428L_N434S; A330L_I332E; or G236A_S239D_A330L_I332E: increased binding (e.g. affinity) to and/or signaling via a human FcγRIIa H131; increased binding (e.g. affinity) to and/or signaling via a human FcγRIIa R131; decreased binding to (e.g. affinity) and/or signaling via human FcγRIIb; an increased ratio of binding to (e.g. affinity) and/or signaling via a human FcγRIIa (H131, R131, or both) versus the ratio of binding to or signaling via (respectively) a human FcγRIIb; increased binding (e.g. affinity) to and/or signaling via a human FcγRIIIa (V158, F158, or both); increased binding (e.g. affinity) to a human C1q; a higher Tm; an improved production titer; an improved signaling in a host cell via a FcγRIIa (H131, R131, or both); increased facilitation of ADCP and/or ADCC by human NK cells and/or human PBMCs when in the presence of antigen-presenting cells; and an improved ability to stimulate moDCs when in an immune complex with antigen.

In the present disclosure, binding of a variant Fc polypeptide or fragment may be described as increased (or “greater than”, or the like) or decreased (or “reduced” or “less than”, or the like) as compared to the binding of a comparator (e.g., to a reference wild-type IgG1 Fc, or to a reference IgG1 Fc that is wild-type except for M428L and N434S mutations or except for M428L and N434A mutations or to a variant IgG1 Fc comprising G236A_A330L_I332E mutations) to a same binding partner. Binding interactions between a variant Fc polypeptide or fragment (or an antibody or polypeptide comprising the same) and a binding partner (e.g. a human FcγR, FcRn, or C1q) can preferably be determined using an electrochemiluminescence assay, more preferably using the Meso Scale Discovery (“MSD”; mesoscale.com) platform. MSD binding assay is similar to ELISA though MSD uses electrochemiluminescence, as opposed to colorimetry, as a detection technique. Other techniques for measuring binding interactions are known and include, for example, ELISA, surface plasmon resonance (SPR), biolayer interferometry (BLI), and the like.

In some embodiments, binding includes affinity, avidity, or both. Affinity refers to the strength of a bond between a binding molecule and its binding partner. In some contexts, binding can include affinity and/or avidity. Unless otherwise indicated, avidity refers to the total binding strength of a molecule to a binding partner, and reflects binding affinity, valency of binding sites (e.g., whether an Fc polypeptide comprises one, two, or more binding sites), and, for example, whether another agent is present that can affect the binding (e.g., a non-competitive inhibitor of the Fc polypeptide).

A binding interaction between a variant molecule of the present disclosure and a binding partner can be expressed in terms of fold-change relative to the binding interaction between a reference molecule and the binding partner. For example, binding of a presently disclosed antibody comprising a variant Fc to a human FcγRIIa may be stronger than the binding of the antibody comprising a wild-type Fc to the human FcγRIIa, and the relative increased strength of the variant can be expressed in terms of fold-change (e.g., linear scale of area-under-the-curve) relative to the reference molecule binding using the same assay. For example, a variant Fc polypeptide or fragment may bind to a FcγRIIa with a 2-fold, 3-fold, 4-fold, or 5-fold greater binding strength than a reference Fc polypeptide or fragment binds to the FcγRIIa. As another example, a variant Fc polypeptide or fragment thereof may bind less strongly to a FcγRIIb as compared to a reference Fc or fragment thereof; e.g., may have a 0.9-fold binding, 0.8-fold binding, 0.7-fold binding, 0.6-fold binding binding, or the like, as compared to the reference Fc polypeptide or fragment thereof. It will be understood that, for example, the expression “2-fold greater binding as compared to the binding of a reference” means a 2-fold increase in binding as compared to the reference.

Moreover, binding of a variant molecule of the present disclosure to two different partner molecules can be described in terms of a ratio, and this ratio can be compared to a like ratio obtained using a reference molecule with the same assay. For example, a variant Fc polypeptide may bind to a human FcγRIIa H131 five times more strongly than it binds to a human FcγRIIb, while a reference wild-type Fc polypeptide binds to FcγRIIa H131 as strongly as it binds to a human FcγRIIb. In this example, the variant Fc polypeptide can be said to have a 5:1 (binding FcγRIIIa H131:binding FcγRIIb) binding ratio, which can be compared to the 1:1 (binding FcγRIIIa H131:binding FcγRIIb) binding ratio of the reference wild-type Fc polypeptide.

Variant molecules of the present disclosure may also be described in terms of ability to induce signaling in a host cell, wherein the host cell expresses or over-expresses one or more FcγR (e.g., FcγRIIa H131, FcγRIIa R131, FcγRIIb, FcγRIIIa F158, or FcγRIIIa V158) and the signaling is induced by binding of the variant molecule to the FcγR. Reporter cells useful for determining signaling include, for example, cells in which NFAT drives expression of a luciferase reporter (e.g., available from Promega®).

Unless stated otherwise, FcγRs, FcRn, and C1q as described herein are human.

In some embodiments, an antibody comprising a variant Fc polypeptide or fragment is preferably capable of inducing one or more of antibody-dependent cell cytotoxicity (ADCC), antibody-dependent cellular phagocytosis (ADCP); and complement-dependent cytotoxicity. Assays for measuring these functions are known.

In some embodiments, a variant Fc polypeptide or fragment (or a polypeptide or antibody comprising the same) preferably has comparable binding to a human FcRn (e.g., at pH 6.0) and/or a comparable in vivo half-life in a mammal as compared to a reference Fc polypeptide, fragment, or antibody, respectively.

In some embodiments, a variant Fc polypeptide or fragment (or a polypeptide or antibody comprising the same) preferably has increased binding to a human FcRn (e.g., at pH 6.0) and/or increased in vivo half-life in a mammal as compared to a reference Fc polypeptide, fragment, or antibody, respectively.

In some embodiments, a variant Fc polypeptide or fragment (or a polypeptide or antibody comprising the same) preferably has a melting temperature (Tm) that is less than 12° C., less than 11° C., less than 10° C., less than 9° C., less than 8° C., less than 7° C., less than 6° C., less than 5° C., less than 4° C., less than 3° C., less than 2° C., or less than 1-C below the Tm of a reference Fc polypeptide or fragment (or polypeptide or antibody comprising the same), or has a Tm that is higher than the Tm of the reference Fc polypeptide or fragment (or polypeptide or antibody comprising the same). In some embodiments, the reference polypeptide or fragment is or comprises a wild-type human Fc polypeptide (or antibody comprising the same).

In some embodiments, a variant Fc polypeptide or fragment (or a polypeptide or antibody comprising the same) has a melting temperature that is higher than the melting temperature of a reference Fc polypeptide or fragment (or polypeptide or antibody comprising the same) that comprises the mutations G236A, A330L, I332E, and, optionally, M428L and N434S, In some embodiments, a variant Fc polypeptide or fragment (or a polypeptide or antibody comprising the same) is preferably capable of being produced in a host cell line (e.g., a CHO cell line) at least about as efficiently (e.g., produces at least about the same titer and/or within less than 0.1-fold, less than 0.09-fold, less than 0.08-fold, less than 0.07-fold, less than 0.06-fold, less than 0.05-fold, less than 0.04-fold, less than 0.03-fold, less than 0.02-fold, or less than 0.02-fold less) as compared to a reference Fc polypeptide or fragment (or polypeptide or antibody comprising the same).

In certain embodiments, a polypeptide is provided that comprises a variant of: (i) an IgG CH2 polypeptide or (ii) an IgG Fc polypeptide or a fragment thereof, wherein the variant comprises an alanine (A) at EU position 236, a valine (V) at EU position 328, and a glutamic acid (E) at EU position 295. In some embodiments, the IgG Fc polypeptide or fragment thereof comprises an (e.g., otherwise wild-type) IgG1 Fc polypeptide or fragment thereof (“GALVQE”). In some embodiments, the polypeptide further comprises the mutations M428L and N434S, or the mutations M428L and N434A, or any other mutation(s) that enhance binding to a human FcRn, such as those described herein. In certain embodiments, the polypeptide is afucosylated.

In certain other embodiments, a polypeptide is provided that comprises a variant of: (i) an IgG hinge-CH2 polypeptide; or (ii) an IgG hinge-Fc polypeptide or a fragment thereof, wherein the variant comprises an alanine (A) at EU position 236, an alanine (A) at EU position 230, and a glutamic acid (E) at EU position 295. In some embodiments, the IgG Fc polypeptide or fragment thereof comprises an (e.g., otherwise wild-type) IgG1 Fc polypeptide or fragment thereof (“GAPAQE”). In some embodiments, the polypeptide further comprises the mutations M428L and N434S, or the mutations M428L and N434A, or any other mutation(s) that enhance binding to a human FcRn, such as those described herein. In certain embodiments, the polypeptide is afucosylated.

In certain other embodiments, a polypeptide is provided that comprises a variant of: an IgG Fc polypeptide or a fragment thereof, wherein the variant comprises an alanine (A) at EU position 236, a proline (P) at EU position 292, and an asparagine (N) at EU position 377. In some embodiments, the IgG Fc polypeptide or fragment thereof comprises an (e.g. otherwise wild-type) IgG1 Fc polypeptide or fragment thereof (“GARPIN”). In some embodiments, the polypeptide further comprises the mutations M428L and N434S, or the mutations M428L and N434A, or any other mutation(s) that enhance binding to a human FcRn, such as those described herein. In certain embodiments, the polypeptide is afucosylated.

In certain other embodiments, a polypeptide is provided that comprises a polypeptide comprising a variant of (i) an IgG CH2 polypeptide or (ii) an IgG Fc polypeptide or a fragment thereof, wherein the variant comprises an alanine (A) at EU position 236, an alanine (A) at EU position 334, and a glutamic acid (E) at EU position 295. In some embodiments, the IgG Fc polypeptide or fragment thereof comprises an (e.g., otherwise wild-type) IgG1 Fc polypeptide or fragment thereof (“GAKAQE”). In some embodiments, the polypeptide further comprises the mutations M428L and N434S, or the mutations M428L and N434A, or any other mutation(s) that enhance binding to a human FcRn, such as those described herein. In certain embodiments, the polypeptide is afucosylated.

In certain other embodiments, a polypeptide is provided that comprises a variant of: (i) an IgG CH2 polypeptide or (ii) an IgG Fc polypeptide or a fragment thereof, wherein the variant comprises a serine (S) at EU position 236, a proline (P) at EU position 292, and a leucine (L) at EU position 300. In some embodiments, the IgG Fc polypeptide or fragment thereof comprises an (e.g., otherwise wild-type) IgG1 Fc polypeptide or fragment thereof (“GSRPYL”). In some embodiments, the polypeptide further comprises the mutations M428L and N434S, or the mutations M428L and N434A, or any other mutation(s) that enhance binding to a human FcRn, such as those described herein. In certain embodiments, the polypeptide is afucosylated.

In certain other embodiments, a polypeptide is provided that comprises a variant of: (i) an IgG CH2 polypeptide or (ii) an IgG Fc polypeptide or a fragment thereof, wherein the variant comprises an alanine (A) at EU position 236, a proline (P) at EU position 292, and a leucine (L) at EU position 300. In some embodiments, the IgG Fc polypeptide or fragment thereof comprises an (e.g., otherwise wild-type) IgG1 Fc polypeptide or fragment thereof (“GARPYL”). In some embodiments, the polypeptide further comprises the mutations M428L and N434S, or the mutations M428L and N434A, or any other mutation(s) that enhance binding to a human FcRn, such as those described herein. In certain embodiments, the polypeptide is afucosylated.

In certain other embodiments, a polypeptide is provided that comprises a variant of: (i) an IgG CH2 polypeptide or (ii) an IgG Fc polypeptide or a fragment thereof, wherein the variant comprises an alanine (A) at EU position 236 and a leucine (L) at EU position 300. In some embodiments, the IgG Fc polypeptide or fragment thereof comprises an (e.g., otherwise wild-type) IgG1 Fc polypeptide or fragment thereof (“GAYL”). In some embodiments, the polypeptide further comprises the mutations M428L and N434S, or the mutations M428L and N434A, or any other mutation(s) that enhance binding to a human FcRn, such as those described herein. In certain embodiments, the polypeptide is afucosylated.

In certain other embodiments, a polypeptide is provided that comprises a variant of: (i) an IgG CH2 polypeptide; or (ii) an IgG Fc polypeptide or a fragment thereof, wherein the variant comprises an alanine (A) at EU position 236, an aspartic acid (D) at EU position 239, and a glutamic acid (E) at EU position 268. In some embodiments, the IgG Fc polypeptide or fragment thereof comprises an (e.g., otherwise wild-type) IgG1 Fc polypeptide or fragment thereof (“GASDHE”). In some embodiments, the polypeptide further comprises the mutations M428L and N434S, or the mutations M428L and N434A, or any other mutation(s) that enhance binding to a human FcRn, such as those described herein. In certain embodiments, the polypeptide is afucosylated.

In some embodiments, the variant, and optionally the polypeptide (e.g. the antibody or Fc fusion comprising the variant), has increased binding to a human FcγRIIa and/or has decreased binding to a human FcγRIIb, as compared to the binding of a reference polypeptide to the human FcγRIIa or the human FcγRIIb, respectively, wherein, optionally, binding is as determined using an electrochemiluminescence assay, further optionally Meso Scale Discovery.

In certain embodiments, the increased binding to a human FcγRIIa comprises more than 1-fold, at least 2-fold, at least 3-fold, at least 4-fold, at least 5-fold, at least 6-fold, at least 7-fold, at least 8-fold, at least 9-fold, or at least 10-fold greater binding to the human FcγRIIa as compared to the binding of a reference polypeptide (optionally comprising a wild-type human IgG (e.g. IgG1) Fc polypeptide or a fragment thereof) to the human FcγRIIa.

In some embodiments, the human FcγRIIa comprises H131 and, optionally, the increased binding to the human FcγRIIa H131 comprises at least 4-fold, at least 5-fold, at least 6-fold, at least 7-fold, at least 8-fold, at least 9-fold, or at least 10-fold greater binding to the human FcγRIIa H131 as compared to the binding of a reference polypeptide (optionally comprising a wild-type human IgG (e.g. IgG1) Fc polypeptide or a fragment thereof) to the human FcγRIIa H131.

In some embodiments, the the human FcγRIIa comprises R131 and, optionally, the increased binding to the human FcγRIIa R131 comprises more than 1-fold, at least 2-fold, at least 3-fold, at least 4-fold, at least 5-fold, at least 6-fold, at least 7-fold, at least 8-fold, at least 9-fold, or at least 10-fold greater binding to the human FcγRIIa R131 as compared to the binding of a reference polypeptide (optionally comprising a wild-type human IgG (e.g. IgG1) Fc polypeptide or a fragment thereof) to the human FcγRIIa R131.

In some embodiments, the decreased binding to a human FcγRIIb comprises less than 0.9-fold, less than 0.8-fold, less than 0.7-fold, less than 0.6-fold, or between 0.5-fold and 0.9-fold, of the binding of a reference polypeptide (optionally comprising a wild-type human IgG (e.g. IgG1) Fc polypeptide or a fragment thereof) to the human FcγRIIb.

In any of the presently disclosed embodiments, (1) a ratio of (i) the binding of the variant or polypeptide to a human FcγRIIa to (ii) the binding of the variant or polypeptide, respectively, to a human FcγRIIb is greater than (2) a ratio of (iii) the binding of a reference polypeptide to the human FcγRIIa to (iv) the binding of the reference polypeptide to the human FcγRIIb, wherein the reference polypeptide optionally comprises a wild-type human IgG (e.g. IgG1) Fc polypeptide or a fragment thereof, wherein, optionally, binding is as determined using an electrochemiluminescence assay, further optionally Meso Scale Discovery. In some embodiments, the human FcγRIIa comprises H131, R131, or both. In some embodiments, the ratio in (1) is more than 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, or at least 14-fold greater than the ratio in (2).

Also provided is a polypeptide comprising a variant of: (i) an IgG CH2 polypeptide or (ii) an IgG Fc polypeptide or a fragment thereof, wherein the variant comprises an alanine (A) at EU position 236 and a leucine (L) at EU position 300. In some embodiments, the IgG Fc polypeptide or fragment thereof comprises an (e.g., otherwise wild-type) IgG1 Fc polypeptide or fragment thereof (“GAYL”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

In some embodiments, the variant, and optionally the polypeptide, has increased binding to a human FcγRIIa as compared to the binding of a reference polypeptide to the human FcγRIIa, wherein, optionally, binding is as determined using an electrochemiluminescence assay, further optionally Meso Scale Discovery.

In some embodiments, the increased binding to a human FcγRIIa comprises at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, or at least 18-fold greater binding to the human FcγRIIa as compared to the binding of a reference polypeptide (optionally comprising a wild-type human IgG (e.g. IgG1) Fc polypeptide or a fragment thereof) to the human FcγRIIa.

In some embodiments, the human FcγRIIa comprises H131 and, optionally, the increased binding to the human FcγRIIa H131 comprises at least 4-fold, at least 5-fold, at least 6-fold, at least 7-fold, at least 8-fold, at least 9-fold, or at least 10-fold, at least 11-fold, at least 12-fold, at least 13-fold, at least 14-fold, at least 15-fold, at least 16-fold, at least 17-fold, or at least 18-fold greater binding to the human FcγRIIa H131 as compared to the binding of a reference polypeptide (optionally comprising a wild-type human IgG (e.g. IgG1) Fc polypeptide or a fragment thereof) to the human FcγRIIa H131.

In some embodiments, the human FcγRIIa comprises R131 and, optionally, the increased binding to the human FcγRIIa R131 comprises at least 4-fold greater binding to the human FcγRIIa R131 as compared to the binding of a reference polypeptide (optionally comprising a wild-type human IgG (e.g. IgG1) Fc polypeptide or a fragment thereof) to the human FcγRIIa R131.

In certain embodiments, (1) a ratio of (i) the binding of the variant or polypeptide to a human FcγRIIa to (ii) the binding of the variant or polypeptide, respectively, to a human FcγRIIb is greater than (2) a ratio of (iii) the binding of a reference polypeptide to the human FcγRIIa to (iv) the binding of the reference polypeptide to the human FcγRIIb, wherein the reference polypeptide comprises a wild-type human IgG Fc polypeptide or a fragment thereof. In certain embodiments, the human FcγRIIa comprises H131, R131, or both. In further embodiments, the ratio in (1) is at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, or at least 17-fold greater than the ratio in (2).

Also provided is a polypeptide comprising a variant of: (i) an IgG CH2 polypeptide or (ii) an IgG Fc polypeptide or a fragment thereof, wherein the variant comprises an alanine (A) at EU position 236, a proline (P) at EU position 292, and a leucine (L) at EU position 300. In some embodiments, the IgG Fc polypeptide or fragment thereof comprises an (e.g. otherwise wild-type) IgG1 Fc polypeptide or fragment thereof (“GARPYL”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

In certain embodiments, the variant, and optionally the polypeptide, has increased binding to a human FcγRIIIa, as compared to the binding of a reference polypeptide to the human FcγRIIIa, wherein, optionally, binding is as determined using an electrochemiluminescence assay, further optionally Meso Scale Discovery.

In some embodiments, the increased binding to a human FcγRIIa comprises at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, or at least 14-fold greater binding to the human FcγRIIa as compared to the binding of a reference polypeptide optionally comprising a wild-type human IgG Fc polypeptide or a fragment thereof to the human FcγRIIa.

In some embodiments, the human FcγRIIa comprises H131 and, optionally, the increased binding to the human FcγRIIa H131 comprises at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, or at least 14-fold greater binding to the human FcγRIIa H131 as compared to the binding of a reference polypeptide optionally comprising a wild-type human IgG Fc polypeptide or a fragment thereof to the human FcγRIIa H131.

In some embodiments, the human FcγRIIa comprises R131 and, optionally, the increased binding to the human FcγRIIa H131 comprises at least 2-fold greater binding to the human FcγRIIa R131 as compared to the binding of a reference polypeptide optionally comprising a wild-type human IgG Fc polypeptide or a fragment thereof to the human FcγRIIa R131.

In certain embodiments, (1) a ratio of (i) the binding of the variant or polypeptide to a human FcγRIIa to (ii) the binding of the variant or polypeptide, respectively, to a human FcγRIIb is greater than (2) a ratio of (iii) the binding of a reference polypeptide to the human FcγRIIa to (iv) the binding of the reference polypeptide to the human FcγRIIb, wherein the reference polypeptide optionally comprises a wild-type human IgG Fc polypeptide or a fragment thereof, wherein, optionally, binding is as determined using an electrochemiluminescence assay, further optionally Meso Scale Discovery. In some embodiments, the human FcγRIIa comprises H131, R131, or both. In some embodiments, the ratio in (1) is at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold greater than the ratio in (2).

In certain embodiments, the variant has increased binding to a human FcγRIIIa, as compared to the binding of a reference polypeptide to the human FcγRIIIa, wherein, optionally, binding is as determined using an electrochemiluminescence assay, further optionally Meso Scale Discovery. In some embodiments, the human FcγRIII comprises V158, F158, or both. In certain further embodiments, the increased binding to a human FcγRIIIa comprises greater than 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0 fold, at least 3.1-fold, at least 3.2-fold, at least 3.3-fold, at least 3.4-fold, at least 3.5-fold, at least 3.6-fold, or at least 3.7-fold greater binding to the human FcγRIIIa as compared to the binding of a reference polypeptide optionally comprising a wild-type human IgG Fc polypeptide or a fragment thereof to the human FcγRIIIa.

In certain embodiments, the variant, and optionally the polypeptide, is capable of binding to a human complement component 1q (C1q), wherein, optionally, binding is as determined using an electrochemiluminescence assay, further optionally Meso Scale Discovery.

Also provided is a polypeptide that comprises a variant of an IgG Fc polypeptide, wherein the variant comprises a serine (S) at EU position 236, a valine (V) at EU position 420, a glutamic acid (E) at EU position 446, and a threonine (T) at EU position 309. In some embodiments, the IgG Fc polypeptide or fragment thereof comprises an (e.g., otherwise wild-type) IgG1 Fc polypeptide or fragment thereof (“GSGVGELT”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

Also provided is a polypeptide that comprises a variant of: (i) an IgG CH2 polypeptide or (ii) an IgG Fc polypeptide, wherein the variant comprises an alanine (A) at EU position 236 and a proline (P) at EU position 292. In some embodiments, the IgG Fc polypeptide or fragment thereof comprises an (e.g., otherwise wild-type) IgG1 Fc polypeptide or fragment thereof (“GARP”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

In certain embodiments, the variant, and optionally the polypeptide, has decreased binding to a human FcγRIIb as compared to the binding of a reference polypeptide to the human FcγRIIb, wherein, optionally, binding is as determined using an electrochemiluminescence assay, further optionally Meso Scale Discovery. In some embodiments, the decreased binding to a human FcγRIIb comprises less than 0.9-fold, less than 0.8-fold, less than 0.7-fold, less than 0.6-fold, less than 0.5-fold, or less than 0.4-fold as compared to the binding of a reference polypeptide optionally comprising a wild-type human IgG Fc polypeptide or a fragment thereof to the human FcγRIIb.

In further embodiments, the variant, and optionally the polypeptide, has increased binding to a human FcγRIIa as compared to the binding of a reference polypeptide to the human FcγRIIa, wherein, optionally, binding is as determined using an electrochemiluminescence assay, further optionally Meso Scale Discovery.

In some embodiments, the increased binding to the human FcγRIIa comprises greater than 1-fold, at least 2-fold, at least 3-fold, at least 4-fold, or at least 5-fold greater binding to the human FcγRIIa as compared to the binding of a reference polypeptide comprising a wild-type human IgG Fc polypeptide or a fragment thereof to the human FcγRIIa.

In certain embodiments, the human FcγRIIa comprises H131, R131, or both.

In some embodiments, (1) a ratio of (i) the binding of the variant or polypeptide to a human FcγRIIa to (ii) the binding of the variant or polypeptide, respectively, to a human FcγRIIb is greater than (2) a ratio of (iii) the binding of a reference polypeptide to the human FcγRIIa to (iv) the binding of the reference polypeptide to the human FcγRIIb, wherein the reference polypeptide optionally comprises a wild-type human IgG Fc polypeptide or a fragment thereof, wherein, optionally, binding is as determined using an electrochemiluminescence assay, further optionally Meso Scale Discovery. In some embodiments, the human FcγRIIa comprises H131, R131, or both. In certain embodiments, the ratio in (1) is at least 2-fold, at least 3-fold, at least 4-fold, at least 5-fold, at least 6-fold, at least 7-fold, at least 8-fold, at least 10-fold, at least 11-fold, or at least 12-fold greater than the ratio in (2).

Also provided is a polypeptide that comprises a variant of: (i) an IgG CH2 polypeptide or (ii) an IgG Fc polypeptide, wherein the variant comprises a proline (P) at EU position 292 and a leucine (L) at EU position 300, and wherein, optionally, variant and, further optionally, the polypeptide has increased binding to a human FcγRIIIa with as compared to the binding of a reference polypeptide to the human FcγRIIIa, wherein, optionally, the binding is as determined using an electrochemiluminescence assay, further optionally Meso Scale Discovery. In some embodiments, the IgG CH2 polypeptide or IgG Fc polypeptide comprises an (e.g., otherwise wild-type) IgG1 CH2 polypeptide or IgG Fc polypeptide (“RPYL”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

In certain embodiments, the human FcγRIIIa comprises V158, F158, or both, and wherein the increased binding to the human FcγRIIIa comprises at least 4-fold, at least 4.5-fold, at least 5-fold, at least 5.1-fold, or at least 5.2-fold greater binding as compared to the binding of a reference polypeptide optionally comprising a wild-type human IgG Fc polypeptide or a fragment thereof to the human FcγRIIa.

Also provided is a polypeptide that comprises a variant of: (i) an IgG CH2 polypeptide or (ii) an IgG Fc polypeptide or a fragment thereof, wherein the variant comprises a leucine (L) at EU position 300. In some embodiments, the IgG CH2 polypeptide or IgG Fc polypeptide or fragment thereof comprises an (e.g., otherwise wild-type) IgG1 Fc polypeptide or fragment thereof (“YL”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

Also provided is a polypeptide that comprises a variant of: an IgG Fc polypeptide or a fragment thereof, wherein the variant comprises a lysine (K) at EU position 345, a serine (S) at EU position 236, tyrosine (Y) at EU position 235, and a glutamic acid (E) at EU position 267. In some embodiments, the IgG Fc polypeptide or fragment thereof comprises an (e.g., otherwise wild-type) IgG1 Fc polypeptide or fragment thereof (“GSEKLYSE”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

Also provided is a polypeptide that comprises a variant of: (i) an IgG hinge-CH2 polypeptide or (ii) an IgG hinge-Fc polypeptide or a fragment thereof, wherein the variant comprises an arginine (R) at EU position 272, a threonine (T) at EU position 309, a tyrosine (Y) at EU position 219, and a glutamic acid (E) at EU position 267. In some embodiments, the IgG hinge-CH2 polypeptide or an IgG hinge-Fc polypeptide or a fragment thereof comprises an (e.g. otherwise wild-type) IgG1 hinge-CH2 polypeptide or IgG hinge-Fc polypeptide or a fragment thereof (“SYSEERLT”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

Also provided is a polypeptide that comprises a variant of: (i) an IgG CH2 polypeptide or (ii) an IgG Fc polypeptide or a fragment thereof, wherein the variant comprises a tyrosine (Y) at EU position 236. In some embodiments, the IgG Fc polypeptide or fragment thereof comprises an (e.g., otherwise wild-type) IgG1 Fc polypeptide or fragment thereof (“GY”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

Also provided is a polypeptide that comprises a variant of: (i) an IgG CH2 polypeptide or (ii) an IgG Fc polypeptide or a fragment thereof, wherein the variant comprises a tryptophan (W) at EU position 236. In some embodiments, the IgG CH2 polypeptide or (ii) an IgG Fc polypeptide or a fragment thereof comprises an (e.g., otherwise wild-type) IgG1 CH2 polypeptide or Fc polypeptide or fragment thereof (“GW”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

Also provided is a polypeptide comprising a variant of: (i) an IgG CH2 polypeptide or (ii) an IgG Fc polypeptide or a fragment thereof, wherein the variant comprises an alanine (A) at EU position 236, wherein the IgG Fc polypeptide or fragment thereof, and optionally the polypeptide, is afucosylated, and wherein, further optionally, the variant comprises a leucine (L) at EU position 330 and a glutamic acid (E) at EU positon 332, wherein, still further optionally, the variant does not comprise an aspartic acid (D) at EU position 239, and, even further optionally, comprises a serine (S) at EU position 239. In some embodiments, the IgG CH2 polypeptide or (ii) an IgG Fc polypeptide or a fragment thereof comprises an (e.g., otherwise wild-type) IgG1 CH2 polypeptide or Fc polypeptide or fragment thereof (“GA-afuc” or “GAALIE-afuc”, respectively). In certain further embodiments, the mutations M428L and N434S, or the mutations M428L and N434A, or any other mutation(s) that enhance binding to a human FcRn, such as those described herein, are present.

Also provided is a polypeptide that comprises a variant of: an IgG Fc polypeptide or a fragment thereof, wherein the variant comprises a leucine (L) at EU position 243, a glutamic acid (E) at EU position 446, a leucine (L) at EU position 396, and a glutamic acid (E) at EU position 267. In some embodiments, the IgG Fc polypeptide or fragment thereof comprises an (e.g., otherwise wild-type) IgG1 Fc polypeptide or fragment thereof (“FLSEPLGE”). In certain further embodiments, the mutations M428L and N434S, or the mutations M428L and N434A, or any other mutation(s) that enhance binding to a human FcRn, such as those described herein, are present. In certain embodiments, the polypeptide is afucoyslated.

Also provided is a polypeptide comprising a variant of: (i) an IgG CH2 polypeptide or (ii) an IgG Fc polypeptide or a fragment thereof, wherein the variant comprises an alanine (A) at EU position 236, an aspartic acid (D) at EU position 239, a glutamic acid (E) and EU position 332, a leucine (L) at EU position 428, and a serine (S) or an alanine (A) at EU position 434. In some embodiments, the IgG Fc polypeptide or fragment thereof comprises an (e.g., otherwise wild-type) IgG1 Fc polypeptide or fragment thereof (“GASDIEMLNS” or “GASDIEMLNA”). In certain embodiments, the polypeptide has increased binding to a human C1q as compared to the binding of a reference polypeptide to the human C1q, wherein, optionally, binding is as determined using an electrochemiluminescence assay, further optionally Meso Scale Discovery. In some embodiments, the increased binding to a human C1q comprises more than 1-fold, at least 1.5-fold, at least 1.75-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0 fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4.0-fold, at least 4.1-fold, or at least 4.15-fold greater binding to the human C1q as compared to the binding of a reference polypeptide comprising a wild-type human IgG Fc polypeptide or a fragment thereof to the human C1q.

In certain of the presently disclosed embodiments, the polypeptide: (i) is capable of binding to a human FcγRIIIa, wherein the human FcγRIIIa comprises a V158, a F158, or both; (ii) is capable of binding to a human FcγRIIIb; (iii) is capable of binding to a human FcRn, optionally at pH 6; (iv) is capable of binding to a human complement component 1q (C1q); (v) has a higher Tm and/or can be produced at a higher titer as compared to (1) a reference polypeptide comprising a human IgG1 Fc polypeptide comprising the amino acid substitutions G236A, S239D, A330L, and I330E (EU numbering), and optionally not comprising any other amino acid substitutions relative to a wild-type human IgG1 Fc polypeptide, (2) a reference polypeptide comprising a human IgG1 Fc polypeptide comprising the amino acid substitutions G236A, A330L, and I330E (EU numbering), and optionally further comprising M428L and N434S mutations and/or M428L and N434A mutations and/or not comprising any other amino acid substitutions and/or not comprising S239D, relative to a wild-type human IgG1 Fc polypeptide, (3) a reference polypeptide comprising a human IgG1 Fc polypeptide comprising the amino acid substitution G236A or G236S (EU numbering), and optionally not comprising any other amino acid substitutions relative to a wild-type human IgG1 Fc polypeptide, and/or (4) a reference polypeptide comprising a human IgG1 Fc polypeptide comprising the amino acid substitutions A330L and I332E (EU numbering), and optionally not comprising any other amino acid substitutions relative to a wild-type human IgG1 Fc polypeptide; (vi) is capable of promoting signaling through a FcγRa in a host cell, wherein, optionally, (a) signaling is optionally increased as compared to the signaling promoted by a reference polypeptide and/or (b) the FcγRa comprises FcγRIIa H131, FcγRIIa R131, FcγRIIIa V158, FcγRIIIa F158, or any combination thereof; (vii) at least when comprised in an antibody, is capable of promoting antibody-dependent cellular cytotoxicity (ADCC); (viii) at least when comprised in an antibody, is capable of promoting antibody-dependent phagocytosis (ADCP); (ix) at least when comprised in an antibody, is capable of promoting complement-dependent cytotoxicity (CDC); (x) at least when comprised in an antibody, is capable of forming an immune complex; or (xi) any combination of (i)-(x).

In any of the presently disclosed embodiments, the variant can further comprise one or more modification that enhances or further enhances binding to a human FcRn as compared to (1) a reference polypeptide that comprises a wild-type human IgG1 Fc polypeptide and/or to (2) the polypeptide without the one or more modification. In some embodiments, the one or more modification that enhances binding to the human FcRn comprises the amino acid substitutions: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; (viii) M428L/N434A; or (ix) any combination of (i)-(viii).

In any of the presently disclosed embodiments, the variant may comprise no any additional mutations as compared to the reference IgG Fc polypeptide or fragment thereof, the IgG hinge-CH2 polypeptide, or the IgG hinge-Fc polypeptide or fragment thereof, respectively. In other embodiments, the variant of an IgG Fc polypeptide comprises, at most: 20, 19, 18, 17, 16, 15, 14, 13, 12, 11, 10, 9, 8, 7, 6, 5, 4, 3, 2, or 1 additional amino acid substitution(s) relative to a wild-type or parental IgG Fc polypeptide, wherein one or more of the additional amino acid substitution(s) optionally comprises a conservative amino acid substitution. In other embodiments, the variant of an IgG Fc polypeptide has at least 85%, at least 86%, at least 87%, at least 88%, at least 89%, at least 90%, at least 91%, at least 92%, at least 93%, at least 94%, at least 95%, at least 96%, or at least 97% identity to a wild-type or parental IgG Fc polypeptide.

In some embodiments, the polypeptide comprises a Fc polypeptide.

In some embodiments, the polypeptide is a monomer comprised in a polypeptide dimer (e.g., a Fc dimer). In some embodiments, the polypeptide is a monomer comprised in a polypeptide homodimer (e.g., a Fc homodimer). In some embodiments, the polypeptide is a monomer comprised in a polypeptide heterodimer (e.g., a Fc heterodimer, optionally comprising a protuberance in a first Fc of the heterodimer and a corresponding cavity in a second Fc of the heterodimer, and/or comprising one or more mutations that provide or contribute to an opposite charge in each of the two Fc monomers (e.g., a positive charge in a region of a first monomer and a negative charge in a corresponding region of a second monomer), and/or comprising a heterologous amino acid sequence in one or both monomers, to promote dimerization of the two Fc monomers).

In come embodiments, a variant Fc polypeptide or fragment is comprised in an antibody. Also provided are antibodies that comprise any of the presently disclosed variants of an Fc polypeptide or fragment of the present disclosure. Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment, provided that a variant Fc polypeptide or fragment as provided herein is comprised in the antibody. Thus, the term “antibody” herein is used in the broadest sense and includes polyclonal and monoclonal antibodies, including intact antibodies as well as functional (antigen-binding) antibody fragments thereof that comprise a Fc polypeptide or fragment; e.g., that comprise an Fc polypeptide and a fragment antigen binding (Fab) fragment, a F(ab′)2 fragment, a Fab′ fragment, a Fv fragment, a recombinant IgG (rIgG) fragment, single chain antibody fragment, including single chain variable fragments (scFv), and a single domain antibody (e.g., sdAb, sdFv, nanobody) fragments; for example, contemplated embodiments include, but are not limited to, intact antibodies; scFv:Fc fusions, scFab:fusions, sdAb:Fc fusions, sdFv:Fc fusions, TriFabs, DART-Fcs, DVD-Igs, Di-diabodies, scFv-Fc, taFv-Fc, scFv-CH3 fusions, scFv-CH2 fusions, CH3 charge-pair antibodies, duobodies, half-antibodies, IgG (HA-Tf-Fv), and the like. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv (provided that a presently disclosed variant of an Fc polypeptide or fragment thereof is present). Unless otherwise stated, the term “antibody” should be understood to encompass functional antibody fragments thereof, provided that a presently disclosed variant of an Fc polypeptide or fragment thereof is present. The term also encompasses intact or full-length antibodies, including antibodies of any class or sub-class, including IgG and sub-classes thereof (IgG1, IgG2, IgG3, IgG4), IgM, IgE, IgA, and IgD.

The terms “V_{L}” or “VL” and “V_{H}” or “VH” refer to the variable binding region from an antibody light chain and an antibody heavy chain, respectively. In certain embodiments, a VL is a kappa (κ) class (also “VK” herein). In certain embodiments, a VL is a lambda (λ) class. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FRT-HCDRT-FR2-HCDR2-FR3-HCDR3-FR4; and an antibody VL comprises four FRs and three CDRs as follows: FR1-LCDRT-FR2-LCDR2-FR3-LCDR3-FR4. In general, the VH and the VL together form the antigen-binding site through their respective CDRs. Numbering of CDR and framework regions may be according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5” ed.; Chothia and Lesk, J Mol. Biol. 196:901-917 (1987)); Lefranc et al., Dev. Comp. Immunol. 27:55, 2003; Honegger and Plückthun, J. Mol. Bio. 309:657-670 (2001)).

In certain embodiments, a polypeptide or antibody of the present disclosure comprises an antigen-binding domain comprising a VH and a VL. In some embodiments, the VH and the VL comprise or consist of the amino acid sequences set forth in SEQ ID NOs.: (i) 26 and 27, respectively; (ii) 28 and 29, respectively; (iii) 30 and 31, respectively; (iv) 30 and 33, respectively; (v) 32 and 31, respectively; (vi) 32 and 33, respectively; (vii) 34 and 35, respectively; (viii) 43 and 44, respectively; (ix) 32 and 46, respectively; (x) 41 and 42, respectively; or (xi) 47 and 48, respectively. In some embodiments, a polypeptide or antibody further comprises a kappa light chain constant domain or a lambda light chain constant domain. In some embodiments, a polypeptide or antibody further comprises a CH1.

In some embodiments, a polypeptide or antibody of the present disclosure comprises an antigen-binding domain comprising a VH and a VL, wherein the the VH and the VL comprise or consist of the amino acid sequences set forth in SEQ ID NOs.:28 and 29 respectively, and the polypeptide or antibody further comprises a variant of an (e.g. IgG1) IgG Fc polypeptide, wherein the variant comprises the following mutations, according to EU numbering: (i) M428L, N434S, G236A, L328V, and Q295E; (ii) M428L, N434S, G236A, R292P, and I377N; (iii) M428L, N434S, G236A, and Y300L; (iv) M428L, N434S, G236A, R292P, and Y300L; (v) M428L, N434S, G236A, L328V, and Q295E, wherein the polypeptide or antibody is afucosylated; (vi) M428L, N434S, G236A, R292P, and I377N, wherein the polypeptide or antibody is afucosylated; (vii) M428L, N434S, G236A, and Y300L, wherein the polypeptide or antibody is afucosylated; or (viii) M428L, N434S, G236A, R292P, and Y300L, wherein the polypeptide or antibody is afucosylated. In some embodiments, the variant of an (e.g. IgG1) IgG Fc polypeptide comprises amino acid substitutions that consist essentially of the substitution mutations in (i), (ii), (iii), (iv), (v), (vi), (vii), or (viii) above. In some embodiments, the antibody comprises a kappa light chain.

In some embodiments, a polypeptide or antibody of the present disclosure comprises an antigen-binding domain comprising a VH and a VL, wherein the the VH and the VL comprise or consist of the amino acid sequences set forth in SEQ ID NOs.:43 and 44, respectively, and the polypeptide or antibody further comprises a variant of an (e.g. IgG1) IgG Fc polypeptide, wherein the variant comprises the following mutations, according to EU numbering: (i) M428L, N434A, G236A, L328V, and Q295E; (ii) M428L, N434A, G236A, R292P, and I377N; (iii) M428L, N434A, G236A, and Y300L; (iv) M428L, N434A, G236A, R292P, and Y300L; (v) M428L, N434A, G236A, L328V, and Q295E, wherein the polypeptide or antibody is afucosylated; (vi) M428L, N434A, G236A, R292P, and I377N, wherein the polypeptide or antibody is afucosylated; (vii) M428L, N434A, G236A, and Y300L, wherein the polypeptide or antibody is afucosylated; or (viii) M428L, N434A, G236A, R292P, and Y300L, wherein the polypeptide or antibody is afucosylated. In some embodiments, the variant of an IgG Fc polypeptide comprises amino acid substitutions that consist essentially of the substitution mutations in (i), (ii), (iii), (iv), (v), (vi), (vii), or (viii) above. In some embodiments, the antibody comprises a kappa light chain.

In some embodiments, a polypeptide or antibody of the present disclosure comprises an antigen-binding domain comprising a VH and a VL, wherein the the VH and the VL comprise or consist of the amino acid sequences set forth in SEQ ID NOs.:43 and 44, respectively, and the polypeptide or antibody further comprises a variant of an (e.g. IgG1) IgG Fc polypeptide, wherein the variant comprises the following mutations, according to EU numbering: (i) M428L, N434S, G236A, L328V, and Q295E; (ii) M428L, N434S, G236A, R292P, and I377N; (iii) M428L, N434S, G236A, and Y300L; (iv) M428L, N434S, G236A, R292P, and Y300L; (v) M428L, N434S, G236A, L328V, and Q295E, wherein the polypeptide or antibody is afucosylated; (vi) M428L, N434S, G236A, R292P, and I377N, wherein the polypeptide or antibody is afucosylated; (vii) M428L, N434S, G236A, and Y300L, wherein the polypeptide or antibody is afucosylated; or (viii) M428L, N434S, G236A, R292P, and Y300L, wherein the polypeptide or antibody is afucosylated. In some embodiments, the variant of an IgG Fc polypeptide comprises amino acid substitutions that consist essentially of the substitution mutations in (i), (ii), (iii), (iv), (v), (vi), (vii), or (viii) above. In some embodiments, the antibody comprises a kappa light chain.

In some embodiments, a polypeptide or antibody of the present disclosure comprises an antigen-binding domain comprising a VH and a VL, wherein the the VH and the VL comprise or consist of the amino acid sequences set forth in SEQ ID NOs.: 43 and 44, respectively, and the polypeptide or antibody further comprises a variant of an (e.g. IgG1) IgG Fc polypeptide, wherein the variant comprises the following mutations, according to EU numbering: (i) M428L, N434A, G236A, L328V, and Q295E; (ii) M428L, N434A, G236A, R292P, and I377N; (iii) M428L, N434A, G236A, and Y300L; (iv) M428L, N434A, G236A, R292P, and Y300L; (v) M428L, N434A, G236A, L328V, and Q295E, wherein the polypeptide or antibody is afucosylated; (vi) M428L, N434A, G236A, R292P, and I377N, wherein the polypeptide or antibody is afucosylated; (vii) M428L, N434A, G236A, and Y300L, wherein the polypeptide or antibody is afucosylated; or (viii) M428L, N434A, G236A, R292P, and Y300L, wherein the polypeptide or antibody is afucosylated. In some embodiments, the variant of an (e.g. IgG1) IgG Fc polypeptide comprises amino acid substitutions that consist essentially of the substitution mutations in (i), (ii), (iii), (iv), (v), (vi), (vii), or (viii) above. In some embodiments, the antibody comprises a kappa light chain.

In certain embodiments, an antibody of the present disclosure comprises an antigen-binding domain from any of the following, non-limiting antibodies: 3F8, 8H9, Abagovomab, Abciximab, Abituzumab, Abrilumab, Actoxumab, Adalimumab, Adecatumumab, Aducanumab, Afasevikumab, Afelimomab, Afutuzumab, Alacizumab pegol, ALD518, Alemtuzumab, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab, Atorolimumab, Avelumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cergutuzumab amunaleukin, Certolizumab pegol, Cetuximab, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Crotedumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dinutuximab, Diridavumab, Domagrozumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erenumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Idarucizumab, Igovomab, IMAB362, Imalumab, Imciromab, Imgatuzumab, Inclacumab, Indatuximab ravtansine, Indusatumab vedotin, Inebilizumab, Infliximab, Inolimomab, Inotuzumab ozogamicin, Intetumumab, Ipilimumab, Iratumumab, Isatuximab, Itolizumab, Ixekizumab, Keliximab, Labetuzumab, Lampalizumab, Lanadelumab, Landogrozumab, Laprituximab emtansine, Lebrikizumab, Lemalesomab, Lendalizumab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, MABp1, Mapatumumab, Margetuximab, Maslimomab, Matuzumab, Mavrilimumab, Mepolizumab, Metelimumab, Milatuzumab, Minretumomab, Mirvetuximab soravtansine, Mitumomab, Mogamulizumab, Monalizumab, Morolimumab, Motavizumab, Moxetumomab pasudotox, Muromonab-CD3, Nacolomab tafenatox, Namilumab, Naptumomab estafenatox, Naratuximah emtansine, Narnatumab, Natalizumab, Navicixizumab, Navivumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Prezalizumab, Priliximab, Pritoxaximab, Pritumumab, PRO 140 aka leronlimab, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab tesirine, Rovelizumab, Ruplizumab, Sacituzumab govitecan, Samalizumab, Sapelizumab, Sarilumab, Satumomab pendetide, Secukinumab, Seribantumab, Setoxaximab, Sevirumab, SGN-CD19A, SGN-CD33A, Sibrotuzumab, Sifalimumab, Siltuximab, Simtuzumab, Siplizumab, Sirukumab, Sofituzumab vedotin, Solanezumab, Solitomab, Sonepcizumab, Sontuzumab, Sotrovimab, Stamulumab, Sulesomab, Suvizumab, Tabalumab, Tacatuzumab tetraxetan, Tadocizumab, Talizumab, Tamtuvetmab, Tanezumab, Taplitumomab paptox, Tarextumab, Tefibazumab, Telimomab aritox, Tenatumomab, Teneliximab, Teplizumab, Teprotumumab, Tesidolumab, Tetulomab, Tezepelumab, TGN1412, Ticilimumab, Tigatuzumab, Tildrakizumab, Timolumab, Tisotumab vedotin, TNX-650, Tocilizumab, Toralizumab, Tosatoxumab, Tositumomab, Tovetumab, Tralokinumab, Trastuzumab, Trastuzumab emtansine, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab mafodotin, Votumumab, Xentuzumab, Zalutumumab, Zanolimumab, Zatuximab, Ziralimumab, Zolimomab aritox, and combinations thereof.

In some embodiments, the polypeptide or antibody comprises an IgG1 isotype. In certain embodiments, the polypeptide or antibody comprises an IgGlm17 allotype, an IgGlm17, 1 allotype, an IgGlm3 allotype, or an IgGlm3, 1 allotype.

In some embodiments, the variant of an IgG Fc polypeptide does not comprise any other amino acid substitution mutations relative to a wild-type or parental IgG Fc polypeptide. In other embodiments, the variant of an IgG Fc polypeptide comprises, at most: 20, 19, 18, 17, 16, 15, 14, 13, 12, 11, 10, 9, 8, 7, 6, 5, 4, 3, 2, or 1 additional amino acid substitution(s) relative to a wild-type or parental IgG Fc polypeptide, wherein one or more of the additional amino acid substitution(s) optionally comprises a conservative amino acid substitution. In other embodiments, the variant of an IgG Fc polypeptide has at least 85%, at least 86%, at least 87%, at least 88%, at least 89%, at least 90%, at least 91%, at least 92%, at least 93%, at least 94%, at least 95%, at least 96%, at least 96%, or at least 97% identity to a wild-type or parental IgG Fc polypeptide.

In certain embodiments, the VH and the variant of an IgG Fc polypeptide are comprised in a heavy chain, and the heavy chain comprises VH-CH1-CH2-CH3. In certain embodiments, the VL is comprised in a light chain that further comprises an (e.g. IgG1) kappa light chain. In other embodiments, the VL is comprised in a light chain that further comprises an (e.g. IgG1) lambda light chain.

A “Fab” (fragment antigen binding) is the part of an antibody that binds to antigen and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the V_{H} and V_{L} antibody domains connected into a single polypeptide chain. In some embodiments, the scFv polypeptide comprises a polypeptide linker disposed between and linking the V_{H} and V_{L} domains that enables the scFv to retain or form the desired structure for antigen binding. Such a peptide linker can be incorporated into a fusion polypeptide using standard techniques well known in the art. Additionally or alternatively, Fv can have a disulfide bond formed between and stabilizing the VH and the VL. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both. scFvs can be comprised in fusion with, or linked or conjugated to, a Fc variant or antibody of the present disclosure.

In some embodiments, an antibody is provided that comprises a variant of an IgG (e.g. of an IgG1) Fc, wherein the variant comprises an alanine (A) at EU position 236, a valine (V) at EU position 328, and a glutamic acid (E) at EU position 295. In certain further embodiments, the mutations M428L and N434S, or the mutations M428L and N434A, or any other mutation(s) that enhance binding to a human FcRn, such as those described herein, are present.

In other embodiments, an antibody is provided that comprises a variant of an IgG (e.g. of an IgG1) Fc, wherein the variant comprises an alanine (A) at EU position 236, an alanine (A) at EU position 230, and a glutamic acid (E) at EU position 295. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises an alanine (A) at EU position 236, a proline (P) at EU position 292, and an asparagine (N) at EU position 377. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises an alanine (A) at EU position 236, an alanine (A) at EU position 334, and a glutamic acid (E) at EU position 295. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises a serine (S) at EU position 236, a proline (P) at EU position 292, and a leucine (L) at EU position 300. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises an alanine (A) at EU position 236 and a leucine (L) at EU position 300. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises an alanine (A) at EU position 236, a proline (P) at EU position 292, and a leucine (L) at EU position 300. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises a serine (S) at EU position 236, a valine (V) at EU position 420, a glutamic acid (E) at EU position 446, and a threonine (T) at EU position 309. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises an alanine (A) at EU position 236 and a leucine (L) at EU position 300. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises a proline (P) at EU position 292 and a leucine (L) at EU position 300. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises a leucine (L) at EU position 300. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises a lysine (K) at EU position 345, a serine (S) at EU position 236, tyrosine (Y) at EU position 235, and a glutamic acid (E) at EU position 267. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises an arginine (R) at EU position 272, a threonine (T) at EU position 309, a tyrosine (Y) at EU position 219, and a glutamic acid (E) at EU position 267. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises a tyrosine (Y) at EU position 236. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises a tryptophan (W) at EU position 236. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises a an alanine (A) at EU position 236, wherein the IgG Fc polypeptide or fragment thereof, and optionally the polypeptide, is afucosylated, and wherein, further optionally, the variant comprises a leucine (L) at EU position 330 and a glutamic acid (E) at EU positon 332, wherein, still further optionally, the variant does not comprise an aspartic acid (D) at EU position 239, and, even further optionally, comprises a serine (S) at EU position 239. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises a leucine (L) at EU position 243, a glutamic acid (E) at EU position 446, a leucine (L) at EU position 396, and a glutamic acid (E) at EU position 267. In certain further embodiments, the mutations M428L and N434S, or the mutations M428L and N434A, or any other mutation(s) that enhance binding to a human FcRn, such as those described herein, are present.

In still other embodiments, an antibody is provided that comprises a variant of an IgG (e.g. of an IgG1) Fc, wherein the variant comprises an alanine (A) at EU position 236, an aspartic acid (D) at EU position 239, a glutamic acid (E) and EU position 332, a leucine (L) at EU position 428, and a serine (S) or an alanine (A) at EU position 434.

In other embodiments, an antibody is provided that comprises a variant of an IgG (e.g. of an IgG1) Fc, wherein the variant comprises an alanine (A) at EU position 236, an aspartic acid (D) at EU position 239, and a glutamic acid (E) at EU position 268.

In some embodiments, the antibody further comprises the mutations M428L and N434S, or the mutations M428L and N434A, or any other mutation(s) that enhance binding to a human FcRn, such as those described herein.

In certain embodiments, the antibody is afucosylated.

In any of the presently disclosed polypeptides or antibodies, the variant Fc or fragment thereof can be derived from an IgG1 isotype, an IgG2 isotype, an IgG3 isotype, or an IgG4 isotype. In certain embodiments, the variant is derived from a human Fc or a fragment thereof, or from a human antibody heavy chain or a fragment thereof. In further embodiments, the variant is derived from a human IgG1 isotype, a human IgG2 isotype, a human IgG3 isotype, or a human IgG3 isotype. In particular embodiments, the variant is derived from a human IgG1 isotype.

A polypeptide, CH2, Fc, CH3, Fc fragment or portion, or antibody, may be of any allotype or combination of allotypes. “Allotype” refers to the allelic variation found among the IgG subclasses. For example, an allotype may comprise Glm1 (or Glm(a)), Glm2 (or Glm(x)), Glm3 (or Glm(f)), Glm17 (or Gm(z))m), Glm27, and/or Glm28 (Glm27 and Glm28 have been described as “alloallotypes”).

The Glm3 and Glm17 allotypes are located at the same position in the CH1 domain (position 214 according to EU numbering). Glm3 comprises R214 (EU), while Glm17 comprises K214 (EU). The Glm1 allotype is located in the CH3 domain (at positions 356 and 358 (EU)) and refers to the replacements E356D and M358L. The Glm2 allotype refers to a replacement of the alanine in position 431 (EU) by a glycine. Glm allotypes, alloallotypes, and features thereof are known in the art and described at, for example, www.imgt.org/IMGTrepertoire/Proteins/allotypes/human/IGH/IGHC/Glm_allotypes.html and Lefranc, M.-P. and Lefranc, G. Human Gm, Km and Am allotypes and their molecular characterization: a remarkable demonstration of polymorphism In: B. Tait, F. Christiansen (Eds.), Immunogenetics, chap. 34, Humana Press, Springer, New York, USA. Methods Mol. Biol. 2012; 882, 635-680. PMID: 22665258, LIGM: 406, the contents and allotypes and allotype information of which are incorporated herein by reference.

The Glm1 allotype may be combined, for example, with the Glm3, Glm17, Glm27, Glm2, and/or Glm28 allotype. In some embodiments, an allotype is Glm3 with no Glm1 (Glm3,−1). In some embodiments, an allotype is Glm17,1 allotype. In some embodiments, an allotype is Glm3,1. In some embodiments, an allotype is Glm17 with no Glm1 (Glm17,−1). Optionally, these allotypes may be combined (or not combined) with the Glm2, Glm27 or Glm28 allotype. For example, an allotype may be Glm17,1,2.

In some embodiments, a polypeptide, CH2, Fe, Fc fragment or portion, or antibody of the present disclosure comprises a Glm3 allotype or a Glm3,1 allotype. In some embodiments, a polypeptide, CH2, Fe, Fc fragment or portion, or antibody of the present disclosure comprises a Glm3 allotype and comprises M428L and N434S or M428L and N434A mutations or any other mutation(s) that enhance binding to a human FcRn, such as those described herein. In some embodiments, a polypeptide, CH2, Fe, Fc fragment or portion, or antibody of the present disclosure comprises a Glm3,1 allotype and comprises M428L and N434S or M428L and N434A mutations or any other mutation(s) that enhance binding to a human FcRn, such as those described herein. In some embodiments, a polypeptide, CH2, Fe, Fc fragment or portion, or antibody of the present disclosure comprises a Glm17, 1 allotype. In some embodiments, a polypeptide, CH2, Fe, Fc fragment or portion, or antibody of the present disclosure comprises a Glm17, 1 allotype and comprises M428L and N434S or M428L and N434A mutations or any other mutation(s) that enhance binding to a human FcRn, as described further herein.

In some embodiments, a polypeptide, CH2, Fe, Fc fragment or portion, or antibody of the present disclosure: (i) is capable of binding to a human FcγRIIIa, wherein the human FcγRIIIa comprises a V158, a F158, or both; (ii) is capable of binding to a human FcγRIIIb; (iii) is capable of binding to a human FcRn, optionally at pH 6; (iv) is capable of binding to a human complement component 1q (C1q), optionally with binding that is increased by more than 1-fold, at least 2-fold, at least 3-fold, or at least 4-fold relative to the binding of an antibody comprising a reference Fc polypeptide (or relative to the binding of a reference polypeptide, CH2, Fe, Fc fragment or portion); (v) has a higher Tm, and/or can be produced at a higher titer, and/or is capable of binding to a human FcγRIIa (optionally, H131 and/or R131) with a higher affinity and/or avidity, and/or is capable of binding to a human FcγRIIb with a lower affinity and/or avidity, as compared to (1) a reference antibody (or a reference polypeptide, CH2, Fe, Fc fragment or portion) that comprises a human IgG1 Fc comprising the amino acid substitutions G236A, S239D, A330L, and I330E (EU numbering), wherein the reference antibody optionally does not comprise any other amino acid substitutions relative in the Fc relative to the wild-type human IgG1 Fe, (2) a reference antibody (or a reference polypeptide, CH2, Fe, Fc fragment or portion) that comprises a human IgG1 Fc comprising the amino acid substitutions G236A, A330L, and I330E (EU numbering), wherein the reference antibody (or a reference polypeptide, CH2, Fe, Fc fragment or portion) optionally (a) further comprises M428L and N434S or M428L and N434A mutations or any other mutation(s) that enhance binding to a human FcRn, such as those described herein, and/or (b) does not comprise any other amino acid substitutions in the Fc relative to the wild-type human IgG1 Fc and/or (c) does not comprise a S239D mutation, (3) a reference antibody (or a reference polypeptide, CH2, Fc, Fc fragment or portion)comprising a human IgG1 Fc that comprises the amino acid substitution G236A or G236S (EU numbering), and optionally does not comprising any other amino acid substitutions om the Fc relative to a wild-type human IgG1 Fc, (4) a reference antibody (or a reference polypeptide, CH2, Fc, Fc fragment or portion) that comprises a human IgG1 Fc comprising the amino acid substitutions A330L and I332E (EU numbering), wherein the reference antibody (or a reference polypeptide, CH2, Fc, Fc fragment or portion) optionally does not comprise any other amino acid substitutions in the Fc relative to the wild-type human IgG1 Fc; and/or (5) a reference antibody (or a reference polypeptide, CH2, Fc, Fc fragment or portion) comprising a wild-type human IgG1 Fc; (vi) is capable of promoting signaling through a FcγRa in a host cell, wherein, optionally, (a) signaling is increased as compared to the signaling promoted by a reference antibody and/or (b) wherein the FcγRa comprises FcγRIIa H131, FcγRIIa R131, FcγRIIIa V158, FcγRIIIa F158, or any combination thereof; (vii) is capable of promoting antibody-dependent cellular cytotoxicity (ADCC); (viii) is capable of promoting antibody-dependent phagocytosis (ADCP); (ix) is capable of promoting complement-dependent cytotoxicity (CDC); (x) is capable of forming an immune complex; or (xi) any combination of (i)-(x).

In some embodiments, the variant Fc of an antibody (or polypeptide) further comprises one or more modification that enhances binding to a human FcRn as compared to (1) a reference antibody comprises a wild-type human IgG1 Fc polypeptide and/or to (2) the antibody without the one or more modification. In certain embodiments, a modification that enhances binding to a human FcRn comprises any one or more of the following substitution mutations: M428L; N434S; N434H; N434A; N434S; M252Y; S254T; T256E; T250Q; P257I Q311I; D376V; T307A; E380A (EU numbering). In certain embodiments, a mutation comprises M428L/N434S (also referred to herein as “MLNS” or “LS”). In certain embodiments, a mutation comprises M428L/N434A (also referred to herein as “MLNA” or “LA”). In certain embodiments, a mutation comprises M252Y/S254T/T256E. In certain embodiments, a mutation comprises T250Q/M428L. In certain embodiments, a mutation comprises P257I/Q311I. In certain embodiments, a mutation comprises P257I/N434H. In certain embodiments, a mutation comprises D376V/N434H. In certain embodiments, a mutation comprises T307A/E380A/N434A. In some embodiments, the one or more modification that enhances binding to the human FcRn comprises the amino acid substitutions: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; (viii) M428L/N434A; or (ix) any combination of (i)-(viii).

In some embodiments, an antibody is provided that comprises, in a human IgG1 heavy chain, the amino acid mutation(s) set forth in any one of (i)-(xvii): (i) G236A, L328V, and Q295E; (ii) G236A, P230A, and Q295E; (iii) G236A, R292P, and I377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E, wherein the numbering of amino acid residues is according to the EU index as set forth in Kabat. In certain further embodiments, the antibody comprises M428L and N434S mutations or or M428L and N434A mutations.

In some embodiments, a polypeptide is provided that comprises at least a portion of a human IgG1 heavy chain comprising the amino acid mutation(s) set forth in any one of (i)-(xvii):
(i) G236A, L328V, and Q295E; (ii) G236A, P230A, and Q295E; (iii) G236A, R292P, and I377N; (iv) G236A, K334A, and Q295E; (v) G236S, R292P, and Y300L; (vi) G236A and Y300L; (vii) G236A, R292P, and Y300L; (viii) G236S, G420V, G446E, and L309T; (ix) G236A and R292P; (x) R292P and Y300L; (xi) G236A and R292P; (xii) Y300L; (xiii) E345K, G236S, L235Y, and S267E; (xiv) E272R, L309T, S219Y, and S267E; (xv) G236Y; (xvi) G236W; (xvii) F243L, G446E, P396L, and S267E, wherein the numbering of amino acid residues is according to the EU index as set forth in Kabat.

In some embodiments, the antibody or polypeptide further comprises one or more mutation that enhances binding to a human FcRn, such as M428L and N434S mutations, or M428L and N434A mutations.

In some embodiments, the antibody or polypeptide comprises the amino acid sequence set forth in any one of SEQ ID NOs.:6-23 and 45, or a variant thereof, e.g., that further comprises one or more mutation that enhances binding to a human FcRn, such as M428L and N434S mutations or M428L and N434A mutations.

In any of the presently disclosed embodiments, a variant Fc of an antibody may not comprise any additional mutations as compared to a reference wild-type IgG Fc.

In any of the presently disclosed embodiments, an antibody is capable of specifically binding to: (i) a target (e.g., an antigen) that is expressed or produced by a pathogen (e.g., virus, bacterium, parasite, fungus) or by a cell infected with the pathogen, wherein, optionally, the pathogen comprises a virus and the virus comprises: a coronavirus; a betacoronavirus; a sarbecovirus; an embecovirus; a nobecovirus; a merbecovirus; a metapneumovirus; a hibecovirus; a SARS-CoV-2; a hepatitis B virus; a hepatitis D virus; a hepatitis C virus; a cytomegalovirus; an influenza A virus; an influenza B virus; a human immunodeficiency virus; a respiratory virus; a respiratory syncytial virus; a zika virus; a rabies virus; a dengue virus; a flavivirus; an ebolavirus; a rhinovirus; or any combination thereof; (ii) a target (e.g., an antigen) that is expressed by, and/or is expressed on a cell surface of, a tumor cell, optionally a cancer cell or a cell of a proliferative or hyperproliferative disorder; (iii) a target (e.g., an antigen) that is associated with an autoimmune disease; (iv) a target (e.g., an antigen) that is associated with a neurodegenerative disease (e.g. tau, alpha-synuclein, amyloid-beta, or the like); (v) an immune system signaling molecule, such as a cytokine; (vi) a target (e.g., an antigen) that is associated with inflammation; (vii) a target (e.g., an antigen) that is associated with a non-infectious disease; or (viii) any combination of (i)-(vii).

In some embodiments, an antibody of the present disclosure specifically binds to any one or more of the following targets: beta-amyloid, 4-1BB, 5AC, 5T4, a-fetoprotein, angiopoietin, AOC3, B7-H3, BAFF, c-MET, c-MYC, C242 antigen, C5, CA-125, CCL11, CCR2, CCR4, CCR5, CD4, CD8, CD11, CD18, CD125, CD140a, CD127, CD15, CD152, CD140, CD19, CD2, CD20, CD22, CD23, CD25, CD27, CD274, CD276, CD28, CD3, CD30, CD33, CD37, CD38, CD4, CD40, CD41, CD44, CD47, CD5, CD51, CD52, CD56, CD6, CD74, CD80, CEA, CFD, CGRP, CLDN, CSF1R, CSF2, CTGF, CTLA-4, CXCR4, CXCR7, DKK1, DLL3, DLL4, DR5, EGFL7, EGFR, EPCAM, ERBB2, ERBB3, FAP, FGF23, FGFR1, GD2, GD3, GDF-8, GPNMB, GUCY2C, HER1, HER2, HGF, HIV-1, HSP90, ICAM-1, IFN-a, IFN-g, IgE, CD221, IGF1, IGF2, IGHE, IL-1, IL2, IL-4, IL-5, IL-6, IL-6R, IL-9, IL-12 IL-15, IL-15R, IL-17, IL-13, IL-18, E,-Ib, IL-22, IL-23, IL23A, integrins, ITGA2, IGTB2, Lewis-Y antigen, LFA-1, LOXL2, LTA, MCP-1, MIF, MS5A1, MUC1, MUC16, MSLN, myostatin, MMP superfamily, NCA-90, NFG, NOGO-A, Notch 1, NRP1, OX-40, OX-40L, P2X superfamily, PCSK9, PD-1, PD-L1, PDCD1, PDGF-R, RANKL, RHD, RON, TRN4, serum albumin, SDC1, SLAMF7, SIRPa, SOST, SHP1, SHP2, STEAP1, TAG-72, TEM1, TIGIT, TFPI, TGF-b, TNF-α, TNF superfamily, TRAIL superfamily, Toll like receptors, WNT superfamily, VEGF-A, VEGFR-1, VWF, cytomegalovirus (CMV), respiratory syncytial virus (RSV), hepatitis B, hepatitis C, influenza A hemagglutinin, rabies virus, HIV virus, herpes simplex virus, and combinations thereof. Other targets or antigens can be found in U.S. Pat. Nos. 9,803,023, 9,663,582, and US20170349662, the contents of which are incorporated herein.

In some embodiments, a cancer is selected from a solid cancer and a hematological malignancy. In certain embodiments, the antigen is selected from a ROR1, CD19, CD20, CD22, EGFR, EGFRvIII, EGP-2, EGP-40, GD2, GD3, HPV E6, HPV E7, HER2, L1-CAM, Lewis A, Lewis Y, MUC1, MUC16, PSCA, PSMA, CD56, CD23, CD24, CD30, CD33, CD37, CD44v7/8, CD38, CD56, CD123, CA125, c-MET, FcRH5, WT1, folate receptor u, VEGF-α, VEGFR1, VEGFR2, IL-13Ru2, IL-IIRa, MAGE-A1, MAGE-A3, MAGE-A4, SSX-2, PRAME, HA-1, Core Binding Factor (CBF), PSA, ephrin A2, ephrin B2, an NKG2D, NY-ESO-1, TAG-72, mesothelin, NY-ESO, α-fetoprotein, CAR15-3, hCG or beta-hcG, 5T4, BCMA, FAP, Carbonic anhydrase 9, BRAF, 02M, ETA, tyrosinase, KRAS, NRAS, MR1, or CEA antigen.

In certain embodiments, a cancer comprises a carcinoma, a sarcoma, a glioma, a lymphoma, a leukemia, a myeloma, or any combination thereof. In certain embodiments, cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

In certain embodiments, a cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenstrom's macroglobulinemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

In certain embodiments, the cancer comprises a solid tumor. In some embodiments, the solid tumor is a sarcoma or a carcinoma. In certain embodiments, the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma. In certain embodiments, the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma). In certain embodiments, the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In any of the presently disclosed embodiments, an antibody can comprise a monoclonal antibody, a chimeric antibody, a humanized antibody, a neutralizing antibody, a human antibody, an IgNAR, a camelid nanobody, or any combination thereof.

The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example. Monoclonal antibodies may also be obtained using methods disclosed in PCT Publication No. WO 2004/076677A2.

Antibodies of the present disclosure include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., Hc domains.

As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody or antigen-binding fragment can be monoclonal.

A “human antibody” is an antibody containing only sequences that are present in an antibody that is produced by a human. However, as used herein, human antibodies may comprise residues or modifications not found in a naturally occurring human antibody (e.g., an antibody that is isolated from a human), including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426.

In certain embodiments, an antibody comprises a multispecific antibody, such as a bispecific antibody, a trispecific antibody, or a tetraspecific antibody. Examples of antibody formats disclosed in Spiess et al., Mol. Immunol. 67(2):95 (2015), and in Brinkmann and Kontermann, mAbs 9(2):182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs (see also Wozniak-Knopp et al., Protein Eng Des Sel. 23(4):289-297 (2010) and Wozniak-Knopp et al., Protein Eng Des Sel. 30(9):657-671 (2017)) rck-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

In certain embodiments, an antibody is comprised in an antibody conjugate.

In certain embodiments, a polypeptide, Fc polypeptide, or antibody: (1) comprises a Fc fusion protein; and/or (2) comprises an Fcab. In some embodiments, the Fc fusion protein further comprises: (i) a receptor domain (e.g. an ectodomain of a receptor protein, or a ligand-binding portion thereof); (ii) a ligand; (iii) a replacement protein (e.g., an enzyme for use in enzyme replacement therapy); or (iv) any combination of (i)-(iii).

In some embodiments, a polypeptide or antibody of the present disclosure is conjugated, linked, or fused to a payload moiety. In certain embodiments, the payload moiety comprises: an antibody or an antigen-binding fragment thereof; a cytotoxic agent (e.g., a chemotherapeutic agent); a detectable compound or detectable label; an oligonucleotide (e.g., an antisense oligonucleotide, a siRNA, or the like); a vector; an agent that stimulates an immune response; a growth factor; or any combination thereof.

Various techniques may be used to couple a payload moiety to a polypeptide or antibody to form a conjugate of the present disclosure. In some embodiments, a conjugate comprises payload molecule that is covalently linked by a linker to the polypeptide or antibody. Linkers used in polypeptide or antibody conjugates comprising cytotoxic or anti-proliferative agents (e.g., antibody drug conjugates) are typically organic compounds that fall into one of two groups, organized according to the mechanism by which the payload molecule is released from the carrier molecule. Cleavable linkers are designed to be selectively degraded or cleaved according to an inherent property of the target cell: three types of cleavable linkers are protease-sensitive linkers (whereby cleavage of the linker, e.g., a linker comprising a valine-citrulline or phenylalanine-lysine dipeptide or a tetrapeptide (e.g., GFLG or ALAL), by proteases present in a tumor cell lysosome releases the payload molecule); pH-sensitive linkers, containing an acid labile group that is selectively hydrolyzed by the lower pH of endosomal and lysosomal compartments, relative to cytosolic pH; and glutathione-sensitive linkers, which comprise a disulfide bridge that is reduced by intracellular glutathione. Non-cleavable linkers rely on non-specific degradation of the conjugate to release the payload molecule.

Specific linkers, payloads, linker chemistries, and related mechanisms and methods are disclosed in Nareshkumar et al., Pharm. Res. 32:3526-3540 (2015), which compositions, methods, and techniques are herein incorporated by reference in their entirety. In certain embodiments, a conjugate comprises a linker is selected from a cleavable linker and a non-cleavable linker. In further embodiments, the linker is a cleavable linker selected from a protease-sensitive linker, a pH-sensitive linker, or a glutathione-sensitive linker. In specific embodiments, a cleavable linker is a protease-sensitive linker comprising a valine-citrulline dipeptide.

A linker may be connected or coupled to the polypeptide antibody using any appropriate technique or mechanism. In some embodiments, a linker comprises a maleimide group (optionally PEGylated) capable of reacting with a reduced disulfide bridge in a hinge region of the antibody or antigen-binding fragment thereof. Other sites on the carrier molecule (i.e., the antibody or antigen-binding fragment thereof) suitable for conjugation to a linker may be introduced or engineered using recombinant techniques, such as introducing cysteine residues or non-natural amino acids for site-specific conjugation. Methods for introducing such modifications include, for example, the method described in Examples 6.3-7 of PCT Publication No. WO 2012/032181.

In some embodiments, a linker further comprises a self-demolishing group, also referred to as a self-immolative group or a self-immolative spacer, to assist in a selective cleavage reaction. In certain embodiments, the self-demolishing group is para-amino benzyl alcohol (PABC).

Click chemistries useful for generating antibody conjugates include those described in Meyer et al., Bioconjug. Chem. 27(12):2791-2807 (2016), and are herein incorporated by reference in their entirety.

In any of the conjugates described herein, the payload molecule may be selected from a therapeutic agent and a detectable indicator. Therapeutic agents suitable for cancer therapy include those disclosed in Parslow et al., Biomedicines 4:14 (2016), which payloads and ADC design principles are hereby incorporated by reference. In certain embodiments, the payload molecule is a therapeutic agent selected from a tubulin-targeting anti-mitotic agent, a peptide-based toxin, a pyrrolobenzodiazepine (PBD) dimer, an antibiotic (e.g., calicheamicin), a pyrimidine synthesis inhibitor (e.g., 5-fluorouracil), an antimetabolite (e.g., methotrexate), a DNA alkylating agent, and a topoisomerase inhibitor (e.g., doxorubicin). In further embodiments, the payload molecule is selected from a mayntansinoid, an auristatin, monomethylauristatin E (MMAE), and monomethylauristatin F (MMAF).

In other embodiments, the payload molecule is a detectable indicator. Detectable indicators suitable for use in conjugates, as well as related labeling strategies and imaging techniques (e.g., PET, MRI, NIR), include those disclosed in Friese and Wu, Mol. Immunol. 67(200):142-152 (2015) and Moek et al., J. Nucl. Med. 58:83S-90S (2017), all of which are hereby incorporated by reference. In certain embodiments, the detectable indicator is selected from a radionuclide, a dye, a radiometal, a fluorescent moiety, an MRI contrast agent, a microbubble, a carbon nanotube, a gold particle, fluorodeoxyglucose, an enzyme, a chromophore, and a radio-opaque marker. In specific embodiments, the detectable indicator is a radionuclide selected from ⁶⁸Ga, ⁶⁴Cu, ⁸⁶Y ⁸⁹Zr, ¹²⁴, ⁹⁹mTc, ¹²³I, ¹¹¹In, ¹⁷⁷Lu, ¹³¹I, ⁷⁶Br, ⁷⁸Zr, ¹⁸F, and ¹²⁴T. In certain such embodiments, an antibody conjugate further comprises a radionuclide chelator selected from maleimide-labeled DOTA, N-hydroxysuccinimide-DOTA, and desferrioxamine (DFO).

In certain embodiments, a payload molecule is covalently linked by a linker to the polypeptide or antibody. In certain embodiments the linker is selected from a cleavable linker and a non-cleavable linker. In certain embodiments the cleavable linker is a protease-sensitive linker, a pH-sensitive linker, or a glutathione-sensitive linker. In certain embodiments, the cleavable linker is a protease-sensitive linker comprising a valine-citrulline dipeptide. In some embodiments, the linker comprises a maleimide group. In certain embodiments, the herein disclosed antibody or antigen-binding fragment thereof comprises a reduced disulfide bridge in a hinge region and the reduced disulfide bridge is coupled to the maleimide group. Also provided herein are embodiments in which the linker further comprises a self-demolishing group, such as, for example, para-amino benzyl alcohol (PABC). In certain embodiments, a polypeptide or antibody conjugate comprises a herein disclosed polypeptide or antibody and a payload molecule that is selected from a therapeutic agent and a detectable indicator. In certain embodiments, the payload molecule is a therapeutic agent selected from a tubulin-targeting anti-mitotic agent, a peptide-based toxin, a pyrrolobenzodiazepine (PBD) dimer, an antibiotic, a pyrimidine synthesis inhibitor, an anti-metabolite, a DNA alkylating agent, and a topoisomerase inhibitor. In certain embodiments, the payload molecule is selected from a mayntansinoid, an auristatin, doxorubicin, calicheamicin, a PBD dimer, monomethylauristatin E (MMAE), and monomethylauristatin F (MMAF). In certain other embodiments, the payload molecule is a detectable indicator. In certain further embodiments, the detectable indicator is selected from a radionuclide, a dye, a radiometal, a fluorescent moiety, an MRI contrast agent, a microbubble, a carbon nanotube, a gold particle, fluorodeoxyglucose, an enzyme, a chromophore, and a radio-opaque marker. In particular embodiments, the detectable indicator is a radionuclide selected from ⁶⁸Ga, ⁶⁴Cu, ⁸⁶Y ⁸⁹Zr, ¹²⁴I, ⁹⁹mTc ¹²³I, ¹¹¹In, ¹⁷⁷Lu, ¹³¹I, ⁷⁶Br, ⁷⁸Zr, ¹⁸F, and ¹²⁴T. In certain embodiments, the conjugate comprises a radionuclide chelator selected from maleimide-labeled DOTA, N-hydroxysuccinimide-DOTA, and desferrioxamine (DFO).

In certain embodiments, a polypeptide or antibody: is afucosylated; has been produced in a host cell that is incapable of fucosylation or that is inhibited in its ability to fucosylate a polypeptide; has been produced under conditions that inhibit fucosylation thereof by a host cell; or any combination thereof.

In certain embodiments, a polypeptide or antibody comprises an amino acid mutation that (1) inhibits fucosylation as compared to a reference polypeptide or antibody, respectively, and/or (2) that abrogates a fucosylation site that is present in the reference polypeptide or antibody, respectively.

In certain embodiments, a polypeptide or antibody comprises a mutation that alters glycosylation, wherein the mutation that alters glycosylation comprises N297A, N297Q, or N297G, and/or the polypeptide or antibody is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin. Cancer Res. 13(6):1875-82 (2007); Huang et al. MAbs 6:1-12 (2018)).

It will be understood that, for example, production in a mammalian cell line can remove one or more C-terminal lysine of an Fc or an antibody heavy chain (see, e.g., Liu et al. mAbs 6(5):1145-1154 (2014)). This lysine corresponds to EU position 447. Accordingly, a polypeptide or antibody of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine (because the C-terminal lysine has been removed), and embodiments where a lysine is the C-terminal residue. In certain embodiments, a composition comprises a plurality of a polypeptide and/or of an antibody of the present disclosure, wherein one or more polypeptide or antibody does not comprise a lysine residue at the C-terminal end of the heavy chain, CH1-CH3, or Fc polypeptide, and wherein one or more polypeptide or antibody comprises a lysine residue at the C-terminal end of the heavy chain, CH1-CH3, or Fc polypeptide.

### Polynucleotides, Vectors, and Host Cells

In another aspect, the present disclosure provides isolated polynucleotides that encode any of the presently disclosed polypeptides, antibodies, fusion proteins, or a portion thereof (e.g., a CH2-CH3, a CH2, a hinge-CH2, a hinge-CH2-CH3, a CH1-CH3, a heavy chain, or the like). In certain embodiments, the polynucleotide is codon-optimized for expression in a host cell. Once a coding sequence is known or identified, codon optimization can be performed using known techniques and tools, e.g., using the GenScript® OptimiumGene™ tool or Gene Synthesis by GeneArt® (ThermoFisher); see also Scholten et al., Clin. Immunol. 119:135, 2006). Codon-optimized sequences include sequences that are partially codon-optimized (i.e., one or a plurality of codons is optimized for expression in the host cell) and those that are fully codon-optimized.

It will also be appreciated that polynucleotides encoding polypeptides (e.g. antibodies) of the present disclosure may possess different nucleotide sequences while still encoding a same polypeptide or antibody due to, for example, the degeneracy of the genetic code, splicing, and the like.

It will be appreciated that in certain embodiments, a polynucleotide encoding a polypeptide or antibody is comprised in a polynucleotide that includes other sequences and/or features for, e.g., expression of the polypeptide or antibody in a host cell. Exemplary features include a promoter sequence, a polyadenylation sequence, a sequence that encodes a signal peptide (e.g., located at the N-terminus of an expressed antibody heavy chain or light chain), or the like.

In any of the presently disclosed embodiments, the polynucleotide can comprise deoxyribonucleic acid (DNA) or ribonucleic acid (RNA). In some embodiments, the RNA comprises messenger RNA (mRNA).

In some embodiments, the polynucleotide comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine.

Vectors are also provided, wherein the vectors comprise or contain a polynucleotide as disclosed herein (e.g., a polynucleotide that encodes a polypeptide or an antibody, or a portion thereof). A vector can comprise any one or more of the vectors disclosed herein. In particular embodiments, a vector is provided that comprises a DNA plasmid construct encoding the polypeptide or antibody, or a portion thereof (e.g., so-called “DMAb”; see, e.g., Muthumani et al., J Infect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the polypeptide or antibody, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the polypeptide or antibody are encoded by a polynucleotide comprised in a single plasmid. In other embodiments, the substituent components of polypeptide or antibody are encoded by a polynucleotide comprised in two or more plasmids (e.g., a first plasmid comprises a polynucleotide encoding a heavy chain, VH, or VH+CH, and a second plasmid comprises a polynucleotide encoding the cognate light chain, VL, or VL+CL). In certain embodiments, a single plasmid comprises a polynucleotide encoding a heavy chain and/or a light chain from two or more polypeptides or antibodies of the present disclosure. An exemplary expression vector is pVaxl, available from Invitrogen®. A DNA plasmid of the present disclosure can be delivered to a subject by, for example, electroporation (e.g., intramuscular electroporation), or with an appropriate formulation (e.g., hyaluronidase).In some embodiments, a vector of the present disclosure comprises a nucleotide sequence encoding a signal peptide. The signal peptide may or may not be present (e.g., can be enzymatically cleaved from) on the mature polypeptide or antibody. In some embodiments, a vector of the present disclosure comprises a polyadenylation signal sequence.

In some embodiments, a vector of the present disclosure comprises a CMV promoter.

In some embodiments, a method is provided that comprises administering to a subject a first polynucleotide (e.g., mRNA) encoding an antibody heavy chain or Fc-containing polypeptide, and administering to the subject a second polynucleotide (e.g., mRNA) encoding the cognate antibody light chain or Fc-containing polypeptide.

In some embodiments, a polynucleotide (e.g., mRNA) is provided that encodes a heavy chain and a light chain of an antibody or antigen binding fragment thereof. In some embodiments, a polynucleotide (e.g., mRNA) is provided that encodes two heavy chains and two light chains of an antibody or antigen binding fragment thereof. See, e.g. Li, JQ., Zhang, ZR., Zhang, H Q. et al. Intranasal delivery of replicating mRNA encoding neutralizing antibody against SARS-CoV-2 infection in mice. Sig Transduct Target Ther 6, 369 (2021). https://doi.org/10.1038/s41392-021-00783-1, the antibody-encoding mRNA constructs, vectors, and related techniques of which are incorporated herein by reference. In some embodiments, a polynucleotide is delivered to a subject via an alphavirus replicon particle (VRP) delivery system. In some embodiments, a replicon comprises a modified VEEV replicon comprising two subgenomic promoters. In some embodiments, a polynucleotide or replicon can translate simultaneously the heavy chain (or VH, or VH+1) and the light chain (or VL, or VL+CL) of an antibody or antigen binding fragment thereof. In some embodiments, a method is provided that comprises delivering to a subject such a polynucleotide or replicon.

In a further aspect, the present disclosure also provides a host cell expressing a polypeptide or antibody according to the present disclosure; or comprising or containing a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NSO cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for polypeptide or antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio Technology 9:545-551 (1991). For example, polypeptides or antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g. for expressing a polypeptide or antibody according to the present disclosure. In such embodiments, the cells may be stably transfected with the vector as described herein. Alternatively, cells may be transiently transfected with a vector according to the present disclosure encoding a polypeptide or antibody as disclosed herein. In any of the presently disclosed embodiments, a polynucleotide may be heterologous to the host cell.

Accordingly, the present disclosure also provides recombinant host cells that heterologously express a polypeptide or antibody of the present disclosure. For example, the cell may be of a species that is different to the species from which the polypeptide or antibody was fully or partially obtained (e.g., CHO cells expressing a human antibody or an engineered human antibody). Moreover, the host cell may impart a post-translational modification (PTM; e.g., glysocylation or fucosylation) on the polypeptide or antibody that is not present in a native (e.g., wild-type) state of the polypeptide or antibody (or in a native state of a parent antibody from which the polypeptide or antibody was engineered or derived). Such a PTM may result in a functional difference (e.g., reduced immunogenicity). Accordingly, a polypeptide or antibody of the present disclosure that is produced by a host cell as disclosed herein may include one or more post-translational modification that is distinct from a reference polypeptide or antibody in its native state (e.g., a wild-type human IgG1 Fc or antibody produced by a CHO cell can comprise a more post-translational modification that is distinct from the Fc or antibody when isolated from the human and/or produced by the native human B cell or plasma cell).

Insect cells useful expressing a polypeptide or antibody of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of a polypeptide or antibody with a partially or fully human glycosylation pattern. See Gemgross, Nat. Biotech. 22:1409-1414 (2004); Li et al., Nat. Biotech. 24:210-215 (2006).

Plant cells can also be utilized as hosts for expressing a polypeptide protein of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER.C6 cell, a YO cell, a Sp2/0 cell, a NSO cell, a human liver cell, a myeloma cell, or a hybridoma cell.

In a related aspect, the present disclosure provides methods for producing a polypeptide or antibody, wherein the methods comprise culturing a host cell of the present disclosure under conditions and for a time sufficient to produce the polypeptide or antibody. Methods useful for isolating and purifying recombinantly produced polypeptides or antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide or antibody. These purification methods may also be employed when isolating an immunogen from its natural environment. Methods for large scale production of one or more of the isolated/recombinant polypeptide or antibody described herein include batch cell culture, which is monitored and controlled to maintain appropriate culture conditions. Purification of soluble polypeptides and antibodies may be performed according to methods described herein and known in the art and that comport with laws and guidelines of domestic and foreign regulatory agencies.

### Compositions

Also provided herein are compositions that comprise any one or more of the presently disclosed polypeptides, antibodies, polynucleotides, vectors, or host cells, singly or in any combination, and can further comprise a pharmaceutically acceptable carrier, excipient, or diluent. Carriers, excipients, and diluents are discussed in further detail herein.

In certain embodiments, a composition comprises a plurality of a polypeptide and/or an antibody of the present disclosure, wherein one or more polypeptide or antibody does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

In certain embodiments, a composition comprises two or more different polypeptides or antibodies according to the present disclosure.

In certain embodiments, a composition comprises afucosylated antibodies or polypeptides.

In certain embodiments, a composition comprises a first vector comprising a first plasmid, and a second vector comprising a second plasmid, wherein the first plasmid comprises a polynucleotide encoding a heavy chain, VH, or VH+CH, and a second plasmid comprises a polynucleotide encoding the cognate light chain, VL, or VL+CL of an antibody. In certain embodiments, a composition comprises a polynucleotide (e.g., mRNA) coupled to a suitable delivery vehicle or carrier. Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanomed Nanobiotechnol. 11(2):e1530 (2019)). Principles, reagents, and techniques for designing appropriate mRNA and and formulating mRNA-LNP and delivering the same are described in, for example, Pardi et al. (J Control Release 217345-351 (2015)); Thess et al. (Mol Ther 23: 1456-1464 (2015)); Thran et al. (EMBO Mol Med 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol:polyethylene glycol lipid), and subcutaneous, intramuscular, intradermal, intravenous, intraperitoneal, and intratracheal administration of the same, are incorporated herein by reference.

### Methods and Uses

Also provided herein are methods of treating a subject using a polypeptide of the present disclosure (e.g., as a fusion protein or carrier molecule), an antibody of the present disclosure (e.g., as a disease-targeting agent or carrier molecule), or a composition comprising the same, wherein the subject has, is believed to have, or is at risk for having a disease or disorder. “Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of a disease or disorder (i.e., in a statistically significant manner). In certain embodiments, therapeutic or prophylactic/preventive benefit includes a reduced duration of hospitalization for treatment of a disease or disorder (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

A “therapeutically effective amount” or “effective amount” of a polypeptide, antibody, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

In some embodiments, the disease or disorder comprises an infectious disease (optionally caused by a viral, bacterial, fungal, or parasitic infection), a cancer, a proliferative disorder, a neurodegenerative disease, an autoimmune disease, or any combination thereof. In further embodiments, an infectious disease comprises: a coronavirus infection, a betacoronavirus infection, a sarbecovirus infection, an embecovirus infection, a nobecovirus infection, a merbecovirus infection, a metapneumovirus infection, a hibecovirus infection, a SARS-CoV-2 infection, a hepatitis B virus infection, a hepatitis D virus infection, an influenza A virus infection, an influenza B virus infection, a human immunodeficiency virus infection, a respiratory virus infection, a respiratory syncytial virus infection, a zika virus infection, a rabies virus infection, a dengue virus infection, a flavivirus infection, an ebolavirus infection, or any combination thereof.

In some embodiments, the disease or disorder comprises a cancer. In certain embodiments, a cancer comprises a solid cancer or a hematological malignancy. In certain embodiments, a cancer comprises a carcinoma, a sarcoma, a glioma, a lymphoma, a leukemia, a myeloma, or any combination thereof. In certain embodiments, cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

In certain embodiments, a cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenstrom's macroglobulinemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

In certain embodiments, the cancer comprises a solid tumor. In some embodiments, the solid tumor is a sarcoma or a carcinoma. In certain embodiments, the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma. In certain embodiments, the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma). In certain embodiments, the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In certain embodiments, treatment is administered as peri-exposure prophylaxis. In certain embodiments, treatment is administered to a subject with mild-to-moderate disease, which may be in an outpatient setting. In certain embodiments, treatment is administered to a subject with moderate-to-severe disease, such as requiring hospitalization.

In some embodiments, a cancer or proliferative disorder comprises a solid tumor. In some embodiments, a cancer or proliferative disorder comprises a hematological malignancy.

Typical routes of administering the presently disclosed compositions thus include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the polypeptide, antibody, polynucleotide, vector, host cell, or composition to the subject.

Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described polypeptide or antibody or antigen-binding fragment in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of a polypeptide, antibody, polynucleotide, vector, host cell, or composition as herein disclosed such that a suitable dosage will be obtained. Typically, this amount is at least 0.01% of the polypeptide or antibody in the composition. When intended for oral administration, this amount may be varied to be between 0.1 and about 70% of the weight of the composition. Certain oral pharmaceutical compositions contain between about 4% and about 75% of the polypeptide or antibody. In certain embodiments, pharmaceutical compositions and preparations according to the present invention are prepared so that a parenteral dosage unit contains between 0.01 to 10% by weight of polypeptide or antibody prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises a polypeptide or antibody as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment thereof in the aqueous delivery system.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount, which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

For example, generally, a therapeutically effective daily dose of an antibody is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polypeptides, polynucleotides, vectors, host cells, and related compositions of the present disclosure, a therapeutically effective dose may be different than for an antibody.

In certain embodiments, a method comprises administering the polypeptide, antibody, polynucleotide, vector, host cell, or composition to the subject at 2, 3, 4, 5, 6, 7, 8, 9, 10 times, or more.

In certain embodiments, a method comprises administering the polypeptide, antibody, polynucleotide, vector, host cell, or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the polypeptide, antibody, polynucleotide, vector, host cell, or composition to the subject a plurality of times, wherein a second or successive administration is performed at about 1 week, about 2 weeks, about 1 month, about 2 months, about 3 months, about 4 months, about 5 months, about 6 months, about 7 months, about 8 months, about 9 months, about 10 months, about 11 months, about 12 months, or more, following a first or prior administration, respectively.

In certain embodiments, a method comprises administering the antibody, antigen-binding fragment, polynucleotide, vector, host cell, or composition at least one time prior to the subject being infected by a pathogen, such as a virus.

Compositions comprising a polypeptide, antibody, polynucleotide, vector, host cell, or composition of the present disclosure may also be administered simultaneously with, prior to, or after administration of one or more other therapeutic agents. Such combination therapy may include administration of a single pharmaceutical dosage formulation which contains a compound of the invention and one or more additional active agents, as well as administration of compositions comprising a polypeptide or antibody of the disclosure and each active agent in its own separate dosage formulation. For example, a polypeptide or antibody as described herein and the other active agent can be administered to the patient together in a single oral dosage composition such as a tablet or capsule, or each agent administered in separate oral dosage formulations. Similarly, a polypeptide or antibody as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising a polypeptide or antibody and one or more additional active agents can be administered at essentially the same time, i.e., concurrently, or at separately staggered times, i.e., sequentially and in any order; combination therapy is understood to include all these regimens.

In certain embodiments, a combination therapy is provided that comprises one or more polypeptide or antibody (or one or more nucleic acid, host cell, vector, or composition) of the present disclosure and one or more anti-inflammatory agent and/or one or more anti-viral agent. In particular embodiments, the one or more anti-inflammatory agent comprises a corticosteroid such as, for example, dexamethasone, prednisone, or the like. In some embodiments, the one or more anti-inflammatory agents comprise a cytokine antagonist such as, for example, an antibody that binds to IL6 (such as siltuximab), or to IL-6R (such as tocilizumab), or to IL-1β, IL-7, IL-8, IL-9, IL-10, FGF, G-CSF, GM-CSF, IFN-γ, IP-10, MCP-1, MIP-1A, MIP1-B, PDGR, TNF-α, or VEGF. In some embodiments, anti-inflammatory agents such as ruxolitinib and/or anakinra are used. In some embodiments, the one or more anti-viral agents comprise nucleotide analogs or nucleotide analog prodrugs such as, for example, remdesivir, sofosbuvir, acyclovir, and zidovudine. In particular embodiments, an anti-viral agent comprises lopinavir, ritonavir, favipiravir, or any combination thereof. In some embodiments, a combination therapy comprises leronlimab. Anti-inflammatory agents for use in a combination therapy of the present disclosure also include non-steroidal anti-inflammatory drugs (NSAIDS). It will be appreciated that in such a combination therapy, the one or more polypeptide or antibody (or one or more nucleic acid, host cell, vector, or composition) and the one or more anti-inflammatory agent and/or one or the more antiviral agent can be administered in any order and any sequence, or together.

In some embodiments, a polypeptide or antibody (or one or more nucleic acid, host cell, vector, or composition) is administered to a subject who has previously received one or more anti-inflammatory agent and/or one or more antiviral agent. In some embodiments, one or more anti-inflammatory agent and/or one or more antiviral agent is administered to a subject who has previously received an antibody (or one or more nucleic acid, host cell, vector, or composition).

In a related aspect, uses of the presently disclosed polypeptides, antibodies, polynucleotides, vectors, host cells, and compositions are provided.

In certain embodiments, a polypeptide, antibody, polynucleotide, vector, host cell, or composition is provided for use in a method of treating a disease or disorder in a subject.

In certain embodiments, a polypeptide, antibody, polynucleotide, vector, host cell, or composition is provided for use in a method of manufacturing or preparing a medicament for treating a disease or disorder in a subject.

Also provided herein are methods for use of a polypeptide, antibody, nucleic acid, vector, cell, or composition of the present disclosure in the diagnosis of a disease or disorder (e.g., in a human subject, or in a sample obtained from a human subject). Methods of diagnosis (e.g., in vitro, ex vivo) may include contacting a polypeptide or antibody with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody complex, in particular following the contacting of an antibody or antibody fragment with a sample. Such a detection step can be performed at the bench, i.e. without any contact to the human or animal body. Examples of detection methods are well-known to the person skilled in the art and include, e.g., ELISA (enzyme-linked immunosorbent assay), including direct, indirect, and sandwich ELISA.

The present disclosure also provides the following non-limiting enumerated Embodiments.

Embodiment 1. A polypeptide comprising a variant of (i) an IgG CH2 polypeptide or (ii) an IgG Fc polypeptide or a fragment thereof, wherein the variant comprises an alanine (A) at EU position 236 and a leucine (L) at EU position 300.

Embodiment 2. The polypeptide of Embodiment 1, wherein the variant, and optionally the polypeptide, has increased binding to a human FcγRIIa as compared to the binding of a reference polypeptide to the human FcγRIIa, wherein, optionally, binding is as determined using an electrochemiluminescence assay, further optionally Meso Scale Discovery.

Embodiment 3. The polypeptide of Embodiment 2, wherein the increased binding to a human FcγRIIa comprises at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, or at least 18-fold greater binding to the human FcγRIIa as compared to the binding of a reference polypeptide comprising a wild-type human IgG Fc polypeptide or a fragment thereof to the human FcγRIIa.

Embodiment 4. The polypeptide of Embodiment 2 or Embodiment 3, wherein the human FcγRIIa comprises H131 and, optionally, the increased binding to the human FcγRIIa H131 comprises at least 4-fold, at least 5-fold, at least 6-fold, at least 7-fold, at least 8-fold, at least 9-fold, or at least 10-fold, at least 11-fold, at least 12-fold, at least 13-fold, at least 14-fold, at least 15-fold, at least 16-fold, at least 17-fold, or at least 18-fold greater binding to the human FcγRIIa H131 as compared to the binding of a reference polypeptide comprising a wild-type human IgG Fc polypeptide or a fragment thereof to the human FcγRIIa H131.

Embodiment 5. The polypeptide of any one of Embodiments 2-4, wherein the human FcγRIIa comprises R131 and, optionally, the increased binding to the human FcγRIIa R131 comprises at least 4-fold greater binding to the human FcγRIIa R131 as compared to the binding of a reference polypeptide comprising a wild-type human IgG Fc polypeptide or a fragment thereof to the human FcγRIIa R131.

Embodiment 6. The polypeptide of any one of Embodiments 2-5, wherein
(1) a ratio of (i) the binding of the variant or polypeptide to a human FcγRIIa to (ii) the binding of the variant or polypeptide, respectively, to a human FcγRIIb
is greater than
(2) a ratio of (iii) the binding of a reference polypeptide to the human FcγRIIa to (iv) the binding of the reference polypeptide to the human FcγRIIb, wherein the reference polypeptide comprises a wild-type human IgG Fc polypeptide or a fragment thereof.

Embodiment 7. The polypeptide of Embodiment 6, wherein the human FcγRIIa comprises H131.

Embodiment 8. The polypeptide of Embodiment 6 or 7, wherein the human FcγRIIa comprises R131.

Embodiment 9. The polypeptide of any one of Embodiments 6-8, wherein the ratio in (1) is at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, or at least 17-fold greater than the ratio in (2).

Embodiment 10. The polypeptide of any one of Embodiments 1-9, further comprising a proline (P) at EU position 292.

Embodiment 11. A polypeptide comprising a variant of (i) an IgG CH2 polypeptide or (ii) an IgG Fc polypeptide or a fragment thereof,
wherein the variant comprises an alanine (A) at EU position 236, a valine (V) at EU position 328, and a glutamic acid (E) at EU position 295.

Embodiment 12. A polypeptide comprising a variant of (i) an IgG hinge-CH2 polypeptide; or (ii) an IgG hinge-Fc polypeptide or a fragment thereof,
wherein the variant comprises an alanine (A) at EU position 236, an alanine (A) at EU position 230, and a glutamic acid (E) at EU position 295.

Embodiment 13. A polypeptide comprising a variant of: an IgG Fc polypeptide or a fragment thereof,
wherein the variant comprises an alanine (A) at EU position 236, a proline (P) at EU position 292, and an asparagine (N) at EU position 377.

Embodiment 14. A polypeptide comprising a variant of (i) an IgG CH2 polypeptide or (ii) an IgG Fc polypeptide or a fragment thereof,
wherein the variant comprises an alanine (A) at EU position 236, an alanine (A) at EU position 334, and a glutamic acid (E) at EU position 295.

Embodiment 15. A polypeptide comprising a variant of (i) an IgG CH2 polypeptide or (ii) an IgG Fc polypeptide or a fragment thereof,
wherein the variant comprises a serine (S) at EU position 236, a proline (P) at EU position 292, and a leucine (L) at EU position 300.

Embodiment 16. The polypeptide of any one of Embodiments 11-15, wherein the variant, and optionally the polypeptide, has increased binding to a human FcγRIIa and/or has decreased binding to a human FcγRIIb,
as compared to the binding of a reference polypeptide to the human FcγRIIa or the human FcγRIIb, respectively,
wherein, optionally, binding is as determined using an electrochemiluminescence assay, further optionally Meso Scale Discovery.

Embodiment 17. The polypeptide of Embodiment 16, wherein the increased binding to a human FcγRIIa comprises more than 1-fold, at least 2-fold, at least 3-fold, at least 4-fold, at least 5-fold, at least 6-fold, at least 7-fold, at least 8-fold, at least 9-fold, or at least 10-fold greater binding to the human FcγRIIa as compared to the binding of a reference polypeptide comprising a wild-type human IgG Fc polypeptide or a fragment thereof to the human FcγRIIa.

Embodiment 18. The polypeptide of Embodiment 16 or Embodiment 17, wherein the human FcγRIIa comprises H131 and, optionally, the increased binding to the human FcγRIIa H131 comprises at least 4-fold, at least 5-fold, at least 6-fold, at least 7-fold, at least 8-fold, at least 9-fold, or at least 10-fold greater binding to the human FcγRIIa H131 as compared to the binding of a reference polypeptide comprising a wild-type human IgG Fc polypeptide or a fragment thereof to the human FcγRIIa H131.

Embodiment 19. The polypeptide of any one of Embodiments 16-18, wherein the human FcγRIIa comprises R131 and, optionally, the increased binding to the human FcγRIIa R131 comprises more than 1-fold, at least 2-fold, at least 3-fold, at least 4-fold, at least 5-fold, at least 6-fold, at least 7-fold, at least 8-fold, at least 9-fold, or at least 10-fold greater binding to the human FcγRIIa R131 as compared to the binding of a reference polypeptide comprising a wild-type human IgG Fc polypeptide or a fragment thereof to the human FcγRIIa R131.

Embodiment 20. The polypeptide of any one of Embodiments 16-19, wherein the decreased binding to a human FcγRIIb comprises less than 0.9-fold, less than 0.8-fold, less than 0.7-fold, less than 0.6-fold, or between 0.5-fold and 0.9-fold, of the binding of a reference polypeptide comprising a wild-type human IgG Fc polypeptide or a fragment thereof to the human FcγRIIb.

Embodiment 21. The polypeptide of any one of Embodiments 1-20, wherein
(1) a ratio of (i) the binding of the variant or polypeptide to a human FcγRIIa to (ii) the binding of the variant or polypeptide, respectively, to a human FcγRIIb
is greater than
(2) a ratio of (iii) the binding of a reference polypeptide to the human FcγRIIa to (iv) the binding of the reference polypeptide to the human FcγRIIb, wherein the reference polypeptide comprises a wild-type human IgG Fc polypeptide or a fragment thereof,
wherein, optionally, binding is as determined using an electrochemiluminescence assay, further optionally Meso Scale Discovery.

Embodiment 22. The polypeptide of Embodiment 21, wherein the human FcγRIIa comprises H131.

Embodiment 23. The polypeptide of Embodiment 21 or 22, wherein the human FcγRIIa comprises R131.

Embodiment 24. The polypeptide of any one of Embodiments 21-23, wherein the ratio in (1) is more than 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, or at least 14-fold greater than the ratio in (2).

Embodiment 25. A polypeptide comprising a variant of: (i) an IgG CH2 polypeptide or (ii) an IgG Fc polypeptide or a fragment thereof, wherein the variant comprises an alanine (A) at EU position 236, a proline (P) at EU position 292, and a leucine (L) at EU position 300.

Embodiment 26. The polypeptide of Embodiment 25, wherein the variant, and optionally the polypeptide, has increased binding to a human FcγRIIIa,
as compared to the binding of a reference polypeptide to the human FcγRIIIa, wherein, optionally, binding is as determined using an electrochemiluminescence assay, further optionally Meso Scale Discovery.

Embodiment 27. The polypeptide of Embodiment 26, wherein the increased binding to a human FcγRIIa comprises at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, or at least 14-fold greater binding to the human FcγRIIa as compared to the binding of a reference polypeptide comprising a wild-type human IgG Fc polypeptide or a fragment thereof to the human FcγRIIa.

Embodiment 28. The polypeptide of Embodiment 26 or Embodiment 27, wherein the human FcγRIIa comprises H131 and, optionally, the increased binding to the human FcγRIIa H131 comprises at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, or at least 14-fold greater binding to the human FcγRIIa H131 as compared to the binding of a reference polypeptide comprising a wild-type human IgG Fc polypeptide or a fragment thereof to the human FcγRIIa H131.

Embodiment 29. The polypeptide of any one of Embodiments 25-28, wherein the human FcγRIIa comprises R131 and, optionally, the increased binding to the human FcγRIIa H131 comprises at least 2-fold greater binding to the human FcγRIIa R131 as compared to the binding of a reference polypeptide comprising a wild-type human IgG Fc polypeptide or a fragment thereof to the human FcγRIIa R131.

Embodiment 30. The polypeptide of any one of Embodiments 25-29, wherein
(1) a ratio of (i) the binding of the variant or polypeptide to a human FcγRIIa to (ii) the binding of the variant or polypeptide, respectively, to a human FcγRIIb
is greater than
(2) a ratio of (iii) the binding of a reference polypeptide to the human FcγRIIa to (iv) the binding of the reference polypeptide to the human FcγRIIb, wherein the reference polypeptide comprises a wild-type human IgG Fc polypeptide or a fragment thereof,
wherein, optionally, binding is as determined using an electrochemiluminescence assay, further optionally Meso Scale Discovery.

Embodiment 31. The polypeptide of Embodiment 30, wherein the human FcγRIIa comprises H131.

Embodiment 32. The polypeptide of Embodiment 30 or 31, wherein the human FcγRIIa comprises R131.

Embodiment 33. The polypeptide of any one of Embodiments 30-32, wherein the ratio in (1) is at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold greater than the ratio in (2).

Embodiment 34. The polypeptide of any one of Embodiments 25-33, wherein the variant has increased binding to a human FcγRIIIa,
as compared to the binding of a reference polypeptide to the human FcγRIIIa,
wherein, optionally, binding is as determined using an electrochemiluminescence assay, further optionally Meso Scale Discovery.

Embodiment 35. The polypeptide of Embodiment 34, wherein the human FcγRIII comprises V158, F158, or both.

Embodiment 36. The polypeptide of Embodiment 34 or 35, wherein the increased binding to a human FcγRIIIa comprises greater than 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0 fold, at least 3.1-fold, at least 3.2-fold, at least 3.3-fold, at least 3.4-fold, at least 3.5-fold, at least 3.6-fold, or at least 3.7-fold greater binding to the human FcγRIIIa as compared to the binding of a reference polypeptide comprising a wild-type human IgG Fc polypeptide or a fragment thereof to the human FcγRIIIa.

Embodiment 37. The polypeptide of any one of Embodiments 25-36, wherein the variant, and optionally the polypeptide, is capable of binding to a human complement component 1q (C1q),
wherein, optionally, binding is as determined using an electrochemiluminescence assay, further optionally Meso Scale Discovery.

Embodiment 38. A polypeptide comprising a variant of an IgG Fc polypeptide, wherein the variant comprises a serine (S) at EU position 236, a valine (V) at EU position 420, a glutamic acid (E) at EU position 446, and a threonine (T) at EU position 309.

Embodiment 39. A polypeptide comprising a variant of: (i) an IgG CH2 polypeptide or (ii) an IgG Fc polypeptide, wherein the variant comprises an alanine (A) at EU position 236 and a proline (P) at EU position 292.

Embodiment 40. The polypeptide of Embodiment 38 or 39, wherein the variant, and optionally the polypeptide, has decreased binding to a human FcγRIIb
as compared to the binding of a reference polypeptide to the human FcγRIIb,
wherein, optionally, binding is as determined using an electrochemiluminescence assay, further optionally Meso Scale Discovery.

Embodiment 41. The polypeptide of Embodiment 40, wherein the decreased binding to a human FcγRIIb comprises less than 0.9-fold, less than 0.8-fold, less than 0.7-fold, less than 0.6-fold, less than 0.5-fold, or less than 0.4-fold as compared to the binding of a reference polypeptide comprising a wild-type human IgG Fc polypeptide or a fragment thereof to the human FcγRIIb.

Embodiment 42. The polypeptide of any one of Embodiments 38-41, wherein the variant, and optionally the polypeptide, has increased binding to a human FcγRIIa
as compared to the binding of a reference polypeptide to the human FcγRIIa,
wherein, optionally, binding is as determined using an electrochemiluminescence assay, further optionally Meso Scale Discovery.

Embodiment 43. The polypeptide of Embodiment 42, wherein the increased binding to the human FcγRIIa comprises greater than 1-fold, at least 2-fold, at least 3-fold, at least 4-fold, or at least 5-fold greater binding to the human FcγRIIa as compared to the binding of a reference polypeptide comprising a wild-type human IgG Fc polypeptide or a fragment thereof to the human FcγRIIa.

Embodiment 44. The polypeptide of Embodiment 42 or 43, wherein the human FcγRIIa comprises H131.

Embodiment 45. The polypeptide of any one of Embodiments 42-44, wherein the human FcγRIIa comprises R131.

Embodiment 46. The polypeptide of any one of Embodiments 38-45, wherein
(1) a ratio of (i) the binding of the variant or polypeptide to a human FcγRIIa to (ii) the binding of the variant or polypeptide, respectively, to a human FcγRIIb
is greater than
(2) a ratio of (iii) the binding of a reference polypeptide to the human FcγRIIa to (iv) the binding of the reference polypeptide to the human FcγRIIb, wherein the reference polypeptide comprises a wild-type human IgG Fc polypeptide or a fragment thereof,
wherein, optionally, binding is as determined using an electrochemiluminescence assay, further optionally Meso Scale Discovery.

Embodiment 47. The polypeptide of Embodiment 46, wherein the human FcγRIIa comprises H131.

Embodiment 48. The polypeptide of Embodiment 46 or 47, wherein the human FcγRIIa comprises R131.

Embodiment 49. The polypeptide of any one of Embodiments 46-48, wherein the ratio in (1) is at least 2-fold, at least 3-fold, at least 4-fold, at least 5-fold, at least 6-fold, at least 7-fold, at least 8-fold, at least 10-fold, at least 11-fold, or at least 12-fold greater than the ratio in (2).

Embodiment 50. A polypeptide comprising a variant of: (i) an IgG CH2 polypeptide or (ii) an IgG Fc polypeptide,
wherein the variant comprises a proline (P) at EU position 292 and a leucine (L) at EU position 300, and wherein, optionally, the variant and, further optionally, the polypeptide, has increased binding to a human FcγRIIIa with as compared to the binding of a reference polypeptide to the human FcγRIIIa, wherein, optionally, the binding is as determined using an electrochemiluminescence assay, further optionally Meso Scale Discovery.

Embodiment 51. The polypeptide of Embodiment 50, wherein the human FcγRIIIa comprises V158, F158, or both, and wherein the increased binding to the human FcγRIIIa comprises at least 4-fold, at least 4.5-fold, at least 5-fold, at least 5.1-fold, or at least 5.2-fold greater binding as compared to the binding of a reference polypeptide comprising a wild-type human IgG Fc polypeptide or a fragment thereof to the human FcγRIIa.

Embodiment 52. A polypeptide comprising a variant of: (i) an IgG CH2 polypeptide or (ii) an IgG Fc polypeptide or a fragment thereof,
wherein the variant comprises a leucine (L) at EU position 300.

Embodiment 53. A polypeptide comprising a variant of: an IgG Fc polypeptide or a fragment thereof,
wherein the variant comprises a lysine (K) at EU position 345, a serine (S) at EU position 236, tyrosine (Y) at EU position 235, and a glutamic acid (E) at EU position 267.

Embodiment 54. A polypeptide comprising a variant of (i) an IgG hinge-CH2 polypeptide or (ii) an IgG hinge-Fc polypeptide or a fragment thereof,
wherein the variant comprises an arginine (R) at EU position 272, a threonine (T) at EU position 309, a tyrosine (Y) at EU position 219, and a glutamic acid (E) at EU position 267.

Embodiment 55. A polypeptide comprising a variant of: (i) an IgG CH2 polypeptide or (ii) an IgG Fc polypeptide or a fragment thereof,
wherein the variant comprises a tyrosine (Y) or a tryptophan (W) at EU position 236.

Embodiment 56. A polypeptide comprising a variant of: (i) an IgG CH2 polypeptide or (ii) an IgG Fc polypeptide or a fragment thereof, wherein the variant comprises an alanine (A) at EU position 236, wherein the IgG CH2 polypeptide of (i) or the IgG Fc polypeptide or fragment thereof of (ii), and optionally the polypeptide, is afucosylated,
wherein, further optionally, the variant comprises a leucine (L) at EU position 330 and a glutamic acid (E) at EU positon 332, wherein, still further optionally, the variant does not comprise an aspartic acid (D) at EU position 239, and, even further optionally, comprises a serine (S) at EU position 239.

Embodiment 57. A polypeptide comprising a variant of: an IgG Fc polypeptide or a fragment thereof,
wherein the variant comprises
(1) a leucine (L) at EU position 243, a glutamic acid (E) at EU position 446, a leucine (L) at EU position 396, and a glutamic acid (E) at EU position 267; or
(2) an alanine (A) at EU position 236, an aspartic acid (D) at EU position 239, a glutamic acid (E) and EU position 332, a leucine (L) at EU position 428, and a serine (S) or an alanine (A) at EU position 434.

Embodiment 58. The polypeptide of any one of Embodiments 50-57, wherein the polypeptide has increased binding to a human C1q
as compared to the binding of a reference polypeptide to the human C1q, wherein, optionally, binding is as determined using an electrochemiluminescence assay, further optionally Meso Scale Discovery.

Embodiment 59. The polypeptide of Embodiment 58, wherein the increased binding to a human C1q comprises more than 1-fold, at least 1.5-fold, at least 1.75-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0 fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4.0-fold, at least 4.1-fold, or at least 4.15-fold greater binding to the human C1q as compared to the binding of a reference polypeptide comprising a wild-type human IgG Fc polypeptide or a fragment thereof to the human C1q.

Embodiment 60. The polypeptide of any one of Embodiments 1-59, which:
(i) is capable of binding to a human FcγRIIIa, wherein the human FcγRIIIa comprises a V158, a F158, or both;
(ii) is capable of binding to a human FcγRIIIb;
(iii) is capable of binding to a human FcRn, optionally at pH 6;
(iv) is capable of binding to a human complement component 1q (C1q);
(v) has a higher Tm and/or can be produced at a higher titer as compared to
   (1) a reference polypeptide comprising a human IgG1 Fc polypeptide comprising the amino acid substitutions G236A, S239D, A330L, and I330E (EU numbering), and optionally not comprising any other amino acid substitutions relative to a wild-type human IgG1 Fc polypeptide,
   (2) a reference polypeptide comprising a human IgG1 Fc polypeptide comprising the amino acid substitutions G236A, A330L, and I330E (EU numbering), and optionally further comprising M428L and N434S mutations or M428L and N434A mutations and/or not comprising any other amino acid substitutions and/or not comprising S239D, relative to a wild-type human IgG1 Fc polypeptide,
   (3) a reference polypeptide comprising a human IgG1 Fc polypeptide comprising the amino acid substitution G236A or G236S (EU numbering), and optionally not comprising any other amino acid substitutions relative to a wild-type human IgG1 Fc polypeptide, and/or
   (4) a reference polypeptide comprising a human IgG1 Fc polypeptide comprising the amino acid substitutions A330L and I332E (EU numbering), and optionally not comprising any other amino acid substitutions relative to a wild-type human IgG1 Fc polypeptide;
(vi) is capable of promoting signaling through a FcγRa in a host cell, wherein, optionally, (a) signaling is optionally increased as compared to the signaling promoted by a reference polypeptide and/or (b) the FcγRa comprises FcγRIIa H131, FcγRIIa R131, FcγRIIIa V158, FcγRIIIa F158, or any combination thereof;
(vii) at least when comprised in an antibody, is capable of promoting antibody-dependent cellular cytotoxicity (ADCC);
(viii) at least when comprised in an antibody, is capable of promoting antibody-dependent phagocytosis (ADCP);
(ix) at least when comprised in an antibody, is capable of promoting complement-dependent cytotoxicity (CDC);
(x) at least when comprised in an antibody, is capable of forming an immune complex; or
(xi) any combination of (i)-(x).

Embodiment 61. The polypeptide of any one of Embodiments 1-60, wherein the polypeptide comprises an antibody and the antibody is capable of any one or more of the following:
(i) increasing specific lysis (e.g. via ADCC) by natural killer cells and/or PBMCs (e.g. expressing F158/V158 or V158/V158 FcγRIIIA) against antigen-expressing target cells, as compared to the antibody comprising a reference Fc polypeptide not comprising the mutation(s) and/or fucosylation state (e.g. the antibody comprising a human IgG1 Fc comprising the mutations G236A, A330L, and I332E);
(ii) increasing ADCP by monocytes (e.g. CD14+ monocytes, optionally expressing F158/V158 FcγRIIA and R131/H131 FcγRIIA or F158/F158 FcγRIIA and R131/H131 FcγRIIA) against antigen-expressing target cells, as compared to the antibody comprising a reference Fc polypeptide not comprising the mutation(s) and/or fucosylation state;
(iii) increasing the percentage of CD83+ cells (e.g. moDCs) and/or increasing expression of CD83 by moDCs in a sample when provided in combination with the antigen, as compared to the antibody comprising a reference Fc polypeptide not comprising the mutation(s) and/or fucosylation state, when provided in combination with the antigen;
(iv) increasing production of one or more cytokine (optionally selected from the group consisting of IL-1β, IFN-γ, IL-6, and TNF-α) by moDCs in a sample when provided in combination with the antigen, as compared to the antibody comprising a reference Fc polypeptide not comprising the mutation(s) and/or fucosylation state, when provided in combination with the antigen; and
(v) increasing the ability of moDCs to stimulate antigen-specific CD4+ T cells when provided to the moDCs in combination with the antigen, as compared to the antibody comprising a reference Fc polypeptide not comprising the mutation(s) and/or fucosylation state, when provided to the moDCs in combination with the antigen, wherein, optionally, (1) the moDCs and the CD4+ T cells are from the same (optionally antigen-vaccinated) subject and/or (2) stimulation of antigen-specific CD4+ T cells is determined by an increase in CD25 expression and/or an increase in proliferation (e.g. as determined by a reduction in CFSE staining over time) and/or an increase in expression of CD69 and/or an increase in expression of NFAT and/or an increase in expression of CD44, by the antigen-specific CD4+ T cells.

Embodiment 62. The polypeptide of any one of Embodiments 1-61, wherein the variant further comprises one or more modification that enhances or further enhances binding to a human FcRn
as compared to (1) a reference polypeptide that comprises a wild-type human IgG1 Fc polypeptide and/or to (2) the polypeptide of any one of Embodiments 1-61 without the one or more modification.

Embodiment 63. The polypeptide of Embodiment 62, wherein the one or more modification that enhances binding to the human FcRn comprises the amino acid substitutions:
(i) M428L/N434S;
(ii) M252Y/S254T/T256E;
(iii) T250Q/M428L;
(iv) P257I/Q311I;
(v) P257I/N434H;
(vi) D376V/N434H;
(vii) T307A/E380A/N434A;
(viii) N434A;
(ix) M428L/N434A; or
(x) any combination of (i)-(ix).

Embodiment 64. The polypeptide of any one of Embodiments 1-63, wherein the variant does not comprise any additional mutations as compared to the reference IgG Fc polypeptide or fragment thereof, the IgG CH2 polypeptide, the IgG hinge-CH2 polypeptide, or the IgG hinge-Fc polypeptide or fragment thereof, respectively.

Embodiment 65. The polypeptide of any one of Embodiments 1-64, which comprises a Fc polypeptide.

Embodiment 66. The polypeptide of any one of Embodiments 1-65, which is a monomer comprised in a polypeptide dimer (e.g., a Fc dimer).

Embodiment 67. The polypeptide of any one of Embodiments 1-66, which is a monomer comprised in a polypeptide homodimer (e.g., a Fc homodimer).

Embodiment 68. The polypeptide of any one of Embodiments 1-66, which is a monomer comprised in a polypeptide heterodimer (e.g., a Fc heterodimer, optionally comprising a protuberance in a first Fc of the heterodimer and a corresponding cavity in a second Fc of the heterodimer, and/or comprising one or more mutations that provide or contribute to an opposite charge in each of the two Fc monomers (e.g., a positive charge in a region of a first monomer and a negative charge in a corresponding region of a second monomer), and/or comprising a heterologous amino acid sequence in one or both monomers, to promote dimerization of the two Fc monomers).

Embodiment 69. The polypeptide of any one of Embodiments 1-68, which is comprised in an antibody.

Embodiment 70. An antibody comprising the polypeptide of any one of Embodiments 1-69.

Embodiment 71. An antibody comprising a variant of an IgG Fc, wherein the variant comprises an alanine (A) at EU position 236 and a leucine (L) at EU position 300.

Embodiment 72. An antibody comprising a variant of an IgG Fc, wherein the variant comprises an alanine (A) at EU position 236, a valine (V) at EU position 328, and a glutamic acid (E) at EU position 295.

Embodiment 73. An antibody comprising a variant of an IgG hinge-Fc, wherein the variant comprises an alanine (A) at EU position 236, an alanine (A) at EU position 230, and a glutamic acid (E) at EU position 295.

Embodiment 74. An antibody comprising a variant of an IgG Fc, wherein the variant comprises an alanine (A) at EU position 236, a proline (P) at EU position 292, and an asparagine (N) at EU position 377.

Embodiment 75. An antibody comprising a variant of an IgG Fc, wherein the variant comprises an alanine (A) at EU position 236, an alanine (A) at EU position 334, and a glutamic acid (E) at EU position 295.

Embodiment 76. An antibody comprising a variant of an IgG Fc, wherein the variant comprises a serine (S) at EU position 236, a proline (P) at EU position 292, and a leucine (L) at EU position 300.

Embodiment 77. An antibody comprising a variant of an IgG Fc, wherein the variant comprises an alanine (A) at EU position 236, a proline (P) at EU position 292, and a leucine (L) at EU position 300.

Embodiment 78. An antibody comprising a variant of an IgG Fc, wherein the variant comprises a serine (S) at EU position 236, a valine (V) at EU position 420, a glutamic acid (E) at EU position 446, and a threonine (T) at EU position 309.

Embodiment 79. An antibody comprising a variant of an IgG Fc, wherein the variant comprises a alanine (A) at EU position 236 and a leucine (L) at EU position 300.

Embodiment 80. An antibody comprising a variant of an IgG Fc, wherein the variant comprises a proline (P) at EU position 292 and a leucine (L) at EU position 300.

Embodiment 81. An antibody comprising a variant of an IgG Fc, wherein the variant comprises a leucine (L) at EU position 300.

Embodiment 82. An antibody comprising a variant of an IgG Fc, wherein the variant comprises a lysine (K) at EU position 345, a serine (S) at EU position 236, tyrosine (Y) at EU position 235, and a glutamic acid (E) at EU position 267.

Embodiment 83. An antibody comprising a variant of an IgG hinge-Fc, wherein the variant comprises an arginine (R) at EU position 272, a threonine (T) at EU position 309, a tyrosine (Y) at EU position 219, and a glutamic acid (E) at EU position 267.

Embodiment 84. An antibody comprising a variant of an IgG Fc, wherein the variant comprises a tyrosine (Y) or a tryptophan (W) at EU position 236.

Embodiment 85. An antibody comprising a variant of an IgG Fc, wherein the variant comprises an alanine (A) at EU position 236, wherein the IgG Fc polypeptide or fragment thereof, and optionally the antibody, is afucosylated,
and wherein, further optionally, the variant comprises a leucine (L) at EU position 330 and a glutamic acid (E) at EU positon 332, wherein, still further optionally, the variant does not comprise an aspartic acid (D) at EU position 239, and, even further optionally, comprises a serine (S) at EU position 239.

Embodiment 86. An antibody comprising a variant of an IgG Fc, wherein the variant comprises:
(1) a leucine (L) at EU position 243, a glutamic acid (E) at EU position 446, a leucine (L) at EU position 396, and a glutamic acid (E) at EU position 267; or
(2) an alanine (A) at EU position 236, an aspartic acid (D) at EU position 239, a glutamic acid (E) and EU position 332, a leucine (L) at EU position 428, and a serine (S) or an alanine (A) at EU position 434.

Embodiment 87. The polypeptide of any one of Embodiments 1-69 or the antibody of any one of Embodiments 70-86, wherein the variant is derived from or comprises an IgG1 isotype, an IgG2 isotype, an IgG3 isotype, or an IgG4 isotype.

Embodiment 88. The polypeptide of any one of Embodiments 1-68 and 87, or the antibody of any one of Embodiments 70-87, wherein the variant is derived from or comprises a human Fc or a fragment thereof, or from a human antibody heavy chain or a fragment thereof.

Embodiment 89. The polypeptide of any one of Embodiments 1-69, 87, and 88, or the antibody of any one of Embodiments 70-88, wherein the variant is derived or comprises from a human IgG1 isotype, a human IgG2 isotype, a human IgG3 isotype, or a human IgG4 isotype.

Embodiment 90. The polypeptide of any one of Embodiments 1-69 and 87-89, or the antibody of any one of Embodiments 70-89, wherein the variant is derived from or comprises a human IgG1 isotype, optionally comprising a Glm3 allotype, a Glm17 allotype, a Glm3,1 allotype, or a Glm17,1 allotype.

Embodiment 91. The antibody of any one of Embodiments 70-90, which:
(i) is capable of binding to a human FcγRIIIa, wherein the human FcγRIIIa comprises a V158, a F158, or both;
(ii) is capable of binding to a human FcγRIIIb;
(iii) is capable of binding to a human FcRn, optionally at pH 6;
(iv) is capable of binding to a human complement component 1q (C1q), optionally with binding that is increased by more than 1-fold, at least 2-fold, at least 3-fold, or at least 4-fold relative to the binding of an antibody comprising a reference Fc polypeptide; (v) has a higher Tm, and/or can be produced at a higher titer, and/or is capable of binding to a human FcγRIIa (optionally, H131 and/or R131) with a higher affinity and/or avidity, and/or is capable of binding to a human FcγRIIb with a lower affinity and/or avidity,
   as compared to
   (1) a reference antibody that comprises a human IgG1 Fc comprising the amino acid substitutions G236A, S239D, A330L, and I330E (EU numbering), wherein the reference antibody optionally does not comprise any other amino acid substitutions relative in the Fc relative to the wild-type human IgG1 Fc,
   (2) a reference antibody that comprises a human IgG1 Fc comprising the amino acid substitutions G236A, A330L, and I330E (EU numbering), wherein the reference antibody optionally (a) further comprises M428L and N434S mutations or M428L and N434A mutations and/or (b) does not comprise any other amino acid substitutions in the Fc relative to the wild-type human IgG1 Fc and/or (c) does not comprise a S239D mutation,
   (3) a reference antibody comprising a human IgG1 Fc that comprises the amino acid substitution G236A or G236S (EU numbering), and optionally does not comprising any other amino acid substitutions om the Fc relative to a wild-type human IgG1 Fc,
   (4) a reference antibody that comprises a human IgG1 Fc comprising the amino acid substitutions A330L and I332E (EU numbering), wherein the reference antibody optionally does not comprise any other amino acid substitutions in the Fc relative to the wild-type human IgG1 Fc; and/or
   (5) a reference antibody comprising a wild-type human IgG1 Fc;
(vi) is capable of promoting signaling through a FcγRa in a host cell, wherein, optionally, (a) signaling is increased as compared to the signaling promoted by a reference antibody and/or (b) wherein the FcγRa comprises FcγRIIa H131, FcγRIIa R131, FcγRIIIa V158, FcγRIIIa F158, or any combination thereof;
(vii) is capable of promoting antibody-dependent cellular cytotoxicity (ADCC);
(viii) is capable of promoting antibody-dependent phagocytosis (ADCP);
(ix) is capable of promoting complement-dependent cytotoxicity (CDC);
(x) is capable of forming an immune complex; or
(xi) any combination of (i)-(x).

Embodiment 92. The antibody of any one of Embodiments 70-91, wherein the antibody is capable of any one or more of the following:
(i) increasing specific lysis (e.g. via ADCC) by natural killer cells and/or PBMCs (e.g. expressing F158/V158 or V158/V158 FcγRIIIA) against antigen-expressing target cells, as compared to the antibody comprising a reference Fc polypeptide not comprising the mutation(s) and/or fucosylation state (e.g. the antibody comprising a human IgG1 Fc comprising the mutations G236A, A330L, and I332E);
(ii) increasing ADCP by monocytes (e.g. CD14+ monocytes, optionally expressing F158/V158 FcγRIIA and R131/H131 FcγRIIA or F158/F158 FcγRIIA and R131/H131 FcγRIIA) against antigen-expressing target cells, as compared to the antibody comprising a reference Fc polypeptide not comprising the mutation(s) and/or fucosylation state;
(iii) increasing the percentage of CD83+ cells (e.g. moDCs) and/or increasing expression of CD83 by moDCs in a sample when provided in combination with the antigen, as compared to the antibody comprising a reference Fc polypeptide not comprising the mutation(s) and/or fucosylation state, when provided in combination with the antigen;
(iv) increasing production of one or more cytokine (optionally selected from the group consisting of IL-1β, IFN-γ, IL-6, and TNF-α) by moDCs in a sample when provided in combination with the antigen, as compared to the antibody comprising a reference Fc polypeptide not comprising the mutation(s) and/or fucosylation state, when provided in combination with the antigen; and
(v) increasing the ability of moDCs to stimulate antigen-specific CD4+ T cells when provided to the moDCs in combination with the antigen, as compared to the antibody comprising a reference Fc polypeptide not comprising the mutation(s) and/or fucosylation state, when provided to the moDCs in combination with the antigen, wherein, optionally, (1) the moDCs and the CD4+ T cells are from the same (optionally antigen-vaccinated) subject and/or (2) stimulation of antigen-specific CD4+ T cells is determined by an increase in CD25 expression and/or an increase in proliferation (e.g. as determined by a reduction in CFSE staining over time) and/or an increase in expression of CD69 and/or an increase in expression of NFAT and/or an increase in expression of CD44, by the antigen-specific CD4+ T cells.

Embodiment 93. The antibody of any one of Embodiments 70-92, wherein the variant further comprises one or more modification that enhances binding to a human FcRn
as compared to (1) a reference antibody comprises a wild-type human IgG1 Fc polypeptide and/or to (2) the antibody of any one of Embodiments 70-92 without the one or more modification.

Embodiment 94. The antibody of Embodiment 93, wherein the one or more modification that enhances binding to the human FcRn comprises the amino acid substitutions:
(i) M428L/N434S;
(ii) M252Y/S254T/T256E;
(iii) T250Q/M428L;
(i) P257I/Q311I;
(ii) P257I/N434H;
(iii) D376V/N434H;
(iv) T307A/E380A/N434A;
(v) M428L/N434A; or
(vi) any combination of (i)-(x).

Embodiment 95. The antibody of any one of Embodiments 70-94, wherein the variant does not comprise any additional mutations as compared to a reference wild-type IgG Fc.

Embodiment 96. The polypeptide of Embodiment 69, or the antibody of any one of Embodiments 70-95, wherein the antibody is capable of specifically binding to:
(i) a target (e.g., an antigen) that is expressed or produced by a pathogen (e.g. virus, bacterium, parasite, fungus) or by a cell infected with the pathogen, wherein, optionally, the pathogen comprises a virus and the virus comprises: a coronavirus; a betacoronavirus; a sarbecovirus; an embecovirus; a nobecovirus; a merbecovirus; a metapneumovirus; a hibecovirus; a SARS-CoV-2; a hepatitis B virus; a hepatitis D virus; an influenza A virus; a cytomegalovirus; a rhinovirus; a hepatitis C virus; an influenza B virus; a human immunodeficiency virus; a respiratory virus; a respiratory syncytial virus; a zika virus; a rabies virus; a dengue virus; a flavivirus; an ebolavirus; or any combination thereof;
(ii) a target (e.g., an antigen) that is expressed by, and/or is expressed on a cell surface of, a tumor cell, optionally a cancer cell or a cell of a proliferative or hyperproliferative disorder;
(iii) a target (e.g., an antigen) that is associated with an autoimmune disease;
(iv) a target (e.g., an antigen) that is associated with a neurodegenerative disease
(v) an immune system signaling molecule, such as a cytokine;
(vi) a target (e.g., an antigen) that is associated with inflammation;
(vii) a target (e.g., an antigen) that is associated with a non-infectious disease; or
(viii) any combination of (i)-(vii).

97. The polypeptide of any one of Embodiments 69, 87-90, and 96, or the antibody of any one of Embodiments 70-96, which comprises a chimeric antibody, a humanized antibody, a neutralizing antibody, a human antibody, an IgNAR, a camelid nanobody, or any combination thereof.

Embodiment 98. The polypeptide of any one of Embodiments 69, 87-90, 96, and 97, or the antibody of any one of Embodiments 70-96, wherein the antibody is a multispecific antibody, such as a bispecific antibody, a trispecific antibody, or a tetraspecific antibody.

Embodiment 99. The polypeptide of any one of Embodiments 69, 87-90, and 96-98, or the antibody of any one of Embodiments 70-98, wherein the antibody is comprised in an antibody conjugate.

Embodiment 100. The polypeptide of any one of Embodiments 1-69, 87-90, and 96-99, or the antibody of any one of Embodiments 70-99, the wherein the polypeptide or the Fc polypeptide: (1) comprises a Fc fusion protein; and/or (2) comprises an Fcab.

Embodiment 101. The polypeptide or antibody of Embodiment 100, wherein the Fc fusion protein further comprises:
(i) a receptor domain (e.g. an ectodomain of a receptor protein, or a ligand-binding portion thereof);
(ii) a ligand;
(iii) a replacement protein; or
(iv) any combination of (i)-(iii).

Embodiment 102. The polypeptide of any one of Embodiments 1-69, 87-90, and 96-101, or the antibody of any one of Embodiments 70-101, which is conjugated, linked, or fused to a payload moiety.

Embodiment 103. The polypeptide or antibody of Embodiment 102, wherein the payload moiety comprises: an antibody or an antigen-binding fragment thereof; a cytotoxic agent (e.g., a chemotherapeutic agent); a detectable compound or detectable label; an oligonucleotide (e.g., an antisense oligonucleotide, a siRNA, or the like); a vector; an agent that stimulates an immune response; a growth factor; or any combination thereof.

Embodiment 104. The polypeptide of any one of Embodiments 1-69, 87-90, and 96-103, or the antibody of any one of Embodiments 70-103, which: is afucosylated; has been produced in a host cell that is incapable of fucosylation or that is inhibited in its ability to fucosylate a polypeptide; has been produced under conditions that inhibit fucosylation thereof by a host cell; or any combination thereof.

Embodiment 105. The polypeptide of any one of Embodiments 1-69, 87-90, and 96-104, or the antibody of any one of Embodiments 70-104, comprising an amino acid mutation that (1) inhibits fucosylation as compared to a reference polypeptide or antibody, respectively, and/or (2) that abrogates a fucosylation site that is present in the reference polypeptide or antibody, respectively.

Embodiment 106. A polynucleotide encoding the polypeptide of any one of Embodiments 1-69, 87-90, and 96-105, or the antibody of any one of Embodiments 70-105.

Embodiment 107. The polynucleotide of Embodiment 106, wherein the polynucleotide is codon optimized for expression by a host cell.

Embodiment 108. A(n e.g. expression) vector comprising the polynucleotide of Embodiment 106 or 107.

Embodiment 109. A host cell comprising the polynucleotide of Embodiment 106 or 107.

Embodiment 110. A host cell comprising the vector of Embodiment 108.

Embodiment 111. A host cell expressing: the polypeptide of any one of Embodiments 1-69, 87-90, and 96-105; and/or the antibody of any one of Embodiments 70-105.

Embodiment 112. A composition comprising:
(i) the polypeptide of any one of Embodiments 1-69, 87-90, and 96-105; and/or
(ii) the antibody of any one of Embodiments 70-105; and/or
(iii) the polynucleotide of Embodiment 106 or 107; and/or
(iv) the vector of Embodiment 108; and/or
(v) the host cell of any one of Embodiments 109-111, and a pharmaceutically acceptable carrier, excipient, or diluent.

Embodiment 113. A method of treating or preventing a disease or disorder in a subject, the method comprising administering to the subject an effective amount of:
(i) the polypeptide of any one of Embodiments 1-69, 87-90, and 96-105;
(ii) the antibody of any one of Embodiments 70-105;
(iii) the polynucleotide of Embodiment 106 or 107;
(iv) the vector of Embodiment 108;
(v) the host cell of any one of Embodiments 109-111; and/or
(vi) the composition of Embodiment 112.

Embodiment 114. The polypeptide of any one of Embodiments 1-69, 87-90, and 96-105, the antibody of any one of Embodiments 70-105, the polynucleotide of Embodiment 106 or 107, the vector of Embodiment 108, the host cell of any one of Embodiments 109-111, and/or the composition of Embodiment 112, for use in treating or preventing a disease or disorder in a subject.

Embodiment 115. The polypeptide of any one of Embodiments 1-69, 87-90, and 96-105, the antibody of any one of Embodiments 70-105, the polynucleotide of Embodiment 106 or 107, the vector of Embodiment 108, the host cell of any one of Embodiments 109-111, and/or the composition of Embodiment 112, for use in the manufacture of a medicament for treating or preventing a disease or disorder in a subject.

Embodiment 116. The method of Embodiment 113 or the polypeptide, antibody, polynucleotide, vector, host cell, and/or composition for use of Embodiment 114 or 115, wherein the disease comprises an infectious disease (optionally caused by a viral, bacterial, fungal, or parasitic infection), a cancer, a proliferative disorder, a neurodegenerative disease, an autoimmune disease, or any combination thereof.

Embodiment 117. The method of Embodiment 116 or the polypeptide, antibody, polynucleotide, vector, host cell, and/or composition for use of Embodiment 116, wherein the infectious disease comprises: a coronavirus infection, a betacoronavirus infection, a sarbecovirus infection, an embecovirus infection, a nobecovirus infection, a merbecovirus infection, a metapneumovirus infection, a hibecovirus infection, a SARS-CoV-2 infection, a hepatitis B virus infection, a hepatitis D virus infection, a hepatitis C virus infection, a cytomegalovirus infection, an influenza A virus infection, an influenza B virus infection, a human immunodeficiency virus infection, a respiratory virus infection, a respiratory syncytial virus infection, a zika virus infection, a rabies virus infection, a dengue virus infection, a flavivirus infection, an ebolavirus infection, or any combination thereof.

TABLE 2
Table of Sequences

| | SEQ | |
|---|---|---|
| Description | ID NO. | Sequence |
| Wild-type human | 1 | ASTKGPSVFPLAPSSKSTSGGTAALGCLVKDYFPEPV |
| IgG1 CH1-CH3 | | TVSWNSGALTSGVHTFPAVLQSSGLYSLSSVVTVPSS |
| (UniProtKB P01857); | | SLGTQTYICNVNHKPSNTKVDKKVEPKSCDKTHTCPP |
| hinge underlined, CH2 | | CPAPELLGGPSVFLFPPKPKDTLMISRTPEVTCVVVDV |
| italicized | | SHEDPEVKFNWYVDGVEVHNAKTKPREEQYNSTYRV |
| | | VSVLTVLHQDWLNGKEYKCKVSNKALPAPIEKTISKA |
| | | KGQPREPQVYTLPPSRDELTKNQVSLTCLVKGFYPSD |
| | | IAVEWESNGQPENNYKTTPPVLDSDGSFFLYSKLTVD |
| | | KSRWQQGNVFSCSVMHEALHNHYTQKSLSLSPGK |
| Wild-type human | 2 | EPKSCDKTHTCPPCPAPELLGGPSVFLFPPKPKDTLMISR |
| | | TPEVTCVVVDVSHEDPEVKFNWYVDGVEVHNAKTKPRE |
| | | EQYNSTYRVVSVLTVLHQDWLNGKEYKCKVSNKALPAPIE |
| | | KTISKAKGQPREPQVYTLPPSRDELTKNQVSLTCLVKG |
| IgG1 hinge-CH2-CH3; | | FYPSDIAVEWESNGQPENNYKTTPPVLDSDGSFFLYSK |
| hinge underlined, CH2 | | LTVDKSRWQQGNVFSCSVMHEALHNHYTQKSLSLSP |
| italicized | | GK |
| Wild-type human | 3 | APELLGGPSVFLFPPKPKDTLMISRTPEVTCVVVDVSH |
| IgG1 CH2 | | EDPEVKFNWYVDGVEVHNAKTKPREEQYNSTYRVVS |
| | | VLTVLHQDWLNGKEYKCKVSNKALPAPIEKTISKAK |
| Wild-type human | 4 | GQPREPQVYTLPPSRDELTKNQVSLTCLVKGFYPSDIA |
| IgG1 CH3 | | VEWESNGQPENNYKTTPPVLDSDGSFFLYSKLTVDKS |
| | | RWQQGNVFSCSVMHEALHNHYTQKSLSLSPGK |
| Wild-type human | 5 | EPKSCDKTHTCPPCPAPELLGGPSVFLFPPKPKDTLMISR |
| IgG1 hinge-CH2; | | TPEVTCVVVDVSHEDPEVKFNWYVDGVEVHNAKTKPRE |
| hinge underlined, CH2 | | EQYNSTYRVVSVLTVLHQDWLNGKEYKCKVSNKALPAPIE |
| italicized | | KTISKAK |
| Human IgG1 CH2- | 6 | APELLAGPSVFLFPPKPKDTLMISRTPEVTCVVVDVSH |
| CH3 amino acid | | EDPEVKFNWYVDGVEVHNAKTKPREEEYNSTYRVVS |
| sequence with G236A, | | VLTVLHQDWLNGKEYKCKVSNKAVPAPIEKTISKAKG |
| L328V, and Q295E | | QPREPQVYTLPPSRDELTKNQVSLTCLVKGFYPSDIAV |
| mutations | | EWESNGQPENNYKTTPPVLDSDGSFFLYSKLTVDKSR |
| | | WQQGNVFSCSVMHEALHNHYTQKSLSLSPGK |
| Human IgG1 hinge- | 7 | EPKSCDKTHTCPPCAAPELLAGPSVFLFPPKPKDTLMIS |
| CH2-CH3 amino acid | | RTPEVTCVVVDVSHEDPEVKFNWYVDGVEVHNAKTK |
| sequence with G236A, | | PREEEYNSTYRVVSVLTVLHQDWLNGKEYKCKVSNK |
| P230A, and Q295E | | ALPAPIEKTISKAKGQPREPQVYTLPPSRDELTKNQVSL |
| mutations | | TCLVKGFYPSDIAVEWESNGQPENNYKTTPPVLDSDG |
| | | SFFLYSKLTVDKSRWQQGNVFSCSVMHEALHNHYTQ |
| | | KSLSLSPGK |
| Human IgG1 CH2- | 8 | APELLAGPSVFLFPPKPKDTLMISRTPEVTCVVVDVSH |
| CH3 amino acid | | EDPEVKFNWYVDGVEVHNAKTKPPEEQYNSTYRVVS |
| sequence with G236A, | | VLTVLHQDWLNGKEYKCKVSNKALPAPIEKTISKAKG |
| R292P, and I377N | | QPREPQVYTLPPSRDELTKNQVSLTCLVKGFYPSDNA |
| mutations | | VEWESNGQPENNYKTTPPVLDSDGSFFLYSKLTVDKS |
| | | RWQQGNVFSCSVMHEALHNHYTQKSLSLSPGK |
| Human IgG1 CH2- | 9 | APELLAGPSVFLFPPKPKDTLMISRTPEVTCVVVDVSH |
| CH3 amino acid | | EDPEVKFNWYVDGVEVHNAKTKPREEEYNSTYRVVS |
| sequence with G236A, | | VLTVLHQDWLNGKEYKCKVSNKALPAPIEATISKAKG |
| K334A, and Q295E | | QPREPQVYTLPPSRDELTKNQVSLTCLVKGFYPSDIAV |
| mutations | | EWESNGQPENNYKTTPPVLDSDGSFFLYSKLTVDKSR |
| | | WQQGNVFSCSVMHEALHNHYTQKSLSLSPGK |
| Human IgG1 CH2- | 10 | APELLSGPSVFLFPPKPKDTLMISRTPEVTCVVVDVSH |
| CH3 amino acid | | EDPEVKFNWYVDGVEVHNAKTKPPEEQYNSTLRVVS |
| sequence with G236S, | | VLTVLHQDWLNGKEYKCKVSNKALPAPIEKTISKAKG |
| R292P, and Y300L | | QPREPQVYTLPPSRDELTKNQVSLTCLVKGFYPSDIAV |
| mutations | | EWESNGQPENNYKTTPPVLDSDGSFFLYSKLTVDKSR |
| | | WQQGNVFSCSVMHEALHNHYTQKSLSLSPGK |
| Human IgG1 CH2- | 11 | APELLAGPSVFLFPPKPKDTLMISRTPEVTCVVVDVSH |
| CH3 amino acid | | EDPEVKFNWYVDGVEVHNAKTKPREEQYNSTLRVVS |
| sequence with G236A | | VLTVLHQDWLNGKEYKCKVSNKALPAPIEKTISKAKG |
| and Y300L mutations | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| Human IgG1 CH2- | 12 | APELLAGPSVFLFPPKPKDTLMISRTPEVTCVVVDVSH |
| CH3 amino acid | | EDPEVKFNWYVDGVEVHNAKTKPPEEQYNSTLRVVS |
| sequence with G236A, | | VLTVLHQDWLNGKEYKCKVSNKALPAPIEKTISKAKG |
| R292P, and Y300L | | QPREPQVYTLPPSRDELTKNQVSLTCLVKGFYPSDIAV |
| mutations | | EWESNGQPENNYKTTPPVLDSDGSFFLYSKLTVDKSR |
| | | WQQGNVFSCSVMHEALHNHYTQKSLSLSPGK |
| Human IgG1 CH2- | 13 | APELLSGPSVFLFPPKPKDTLMISRTPEVTCVVVDVSH |
| CH3 amino acid | | EDPEVKFNWYVDGVEVHNAKTKPREEQYNSTYRVVS |
| sequence with G236S, | | VLTVTHQDWLNGKEYKCKVSNKALPAPIEKTISKAKG |
| G420V, G446E, and | | QPREPQVYTLPPSRDELTKNQVSLTCLVKGFYPSDIAV |
| L309T mutations | | EWESNGQPENNYKTTPPVLDSDGSFFLYSKLTVDKSR |
| | | WQQVNVFSCSVMHEALHNHYTQKSLSLSPEK |
| Human IgG1 CH2- | 14 | APELLAGPSVFLFPPKPKDTLMISRTPEVTCVVVDVSH |
| CH3 amino acid | | EDPEVKFNWYVDGVEVHNAKTKPPEEQYNSTYRVVS |
| sequence with G236A | | VLTVLHQDWLNGKEYKCKVSNKALPAPIEKTISKAKG |
| and R292P mutations | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| Human IgG1 CH2- | 15 | APELLGGPSVFLFPPKPKDTLMISRTPEVTCVVVDVSH |
| CH3 amino acid | | EDPEVKFNWYVDGVEVHNAKTKPPEEQYNSTLRVVS |
| sequence with R292P | | VLTVLHQDWLNGKEYKCKVSNKALPAPIEKTISKAKG |
| and Y300L mutations | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| Human IgG1 CH2- | 16 | APELLAGPSVFLFPPKPKDTLMISRTPEVTCVVVDVSH |
| CH3 amino acid | | EDPEVKFNWYVDGVEVHNAKTKPPEEQYNSTYRVVS |
| sequence with G236A | | VLTVLHQDWLNGKEYKCKVSNKALPAPIEKTISKAKG |
| and R292P mutations | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| Human IgG1 CH2- | 17 | APELLGGPSVFLFPPKPKDTLMISRTPEVTCVVVDVSH |
| CH3 amino acid | | EDPEVKFNWYVDGVEVHNAKTKPREEQYNSTLRVVS |
| sequence with Y300L | | VLTVLHQDWLNGKEYKCKVSNKALPAPIEKTISKAKG |
| mutation | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| Human IgG1 CH2- | 18 | APELYSGPSVFLFPPKPKDTLMISRTPEVTCVVVDVEH |
| CH3 amino acid | | EDPEVKFNWYVDGVEVHNAKTKPREEQYNSTYRVVS |
| sequence with E345K, | | VLTVLHQDWLNGKEYKCKVSNKALPAPIEKTISKAKG |
| G236S, L235Y, and | | QPRKPQVYTLPPSRDELTKNQVSLTCLVKGFYPSDIAV |
| S267E mutations | | EWESNGQPENNYKTTPPVLDSDGSFFLYSKLTVDKSR |
| | | WQQGNVFSCSVMHEALHNHYTQKSLSLSPGK |
| Human IgG1 hinge- | 19 | EPKYCDKTHTCPPCPAPELLGGPSVFLFPPKPKDTLMIS |
| CH2-CH3 amino acid | | RTPEVTCVVVDVEHEDPRVKFNWYVDGVEVHNAKT |
| sequence with E272R, | | KPREEQYNSTYRVVSVLTVTHQDWLNGKEYKCKVSN |
| L309T, S219Y, and | | KALPAPIEKTISKAKGQPREPQVYTLPPSRDELTKNQV |
| S267E mutations | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGK |
| Human IgG1 CH2- | 20 | APELLYGPSVFLFPPKPKDTLMISRTPEVTCVVVDVSH |
| CH3 amino acid | | EDPEVKFNWYVDGVEVHNAKTKPREEQYNSTYRVVS |
| sequence with G236Y | | VLTVLHQDWLNGKEYKCKVSNKALPAPIEKTISKAKG |
| mutation | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| Human IgG1 CH2- | 21 | APELLWGPSVFLFPPKPKDTLMISRTPEVTCVVVDVSH |
| CH3 amino acid | | EDPEVKFNWYVDGVEVHNAKTKPREEQYNSTYRVVS |
| sequence with G236W | | VLTVLHQDWLNGKEYKCKVSNKALPAPIEKTISKAKG |
| mutation | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| Human IgG1 CH2- | 22 | APELLGGPSVFLLPPKPKDTLMISRTPEVTCVVVDVEH |
| CH3 amino acid | | EDPEVKFNWYVDGVEVHNAKTKPREEQYNSTYRVVS |
| sequence with F243L, | | VLTVLHQDWLNGKEYKCKVSNKALPAPIEKTISKAKG |
| G446E, P396L, and | | QPREPQVYTLPPSRDELTKNQVSLTCLVKGFYPSDIAV |
| S267E mutations | | EWESNGQPENNYKTTPLVLDSDGSFFLYSKLTVDKSR |
| | | WQQGNVFSCSVMHEALHNHYTQKSLSLSPEK |
| Human IgG1 CH2- | 23 | APELLAGPSVFLFPPKPKDTLMISRTPEVTCVVVDVSH |
| CH3 amino acid | | EDPEVKFNWYVDGVEVHNAKTKPREEQYNSTYRVVS |
| sequence with G236A | | VLTVLHQDWLNGKEYKCKVSNKALPAPIEKTISKAKG |
| mutation | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| [reserved] | 24 | |
| [reserved] | 25 | |
| HBC34-v35 VH | 26 | ELQLVESGGGWVQPGGSQRLSCAASGRIFRSFYMSWV |
| amino acid sequence | | RQAPGKGLEWVATINQDGSEKLYVDSVKGRFTISRDN |
| | | AKNSLFLQMNNLRVEDTAVYYCAAWSGNSGGMDV |
| | | WGQGTTVSVSS |
| HBC34-v35 VL amino | 27 | SYELTQPPSVSVSPGQTVSIPCSGDKLGNKNVAWFQH |
| acid sequence | | KPGQSPVLVIYEVKYRPSGIPERFSGSNSGNTATLTISG |
| | | TQAMDEAAYFCQTFDSTTVVFGGGTRLTVL |
| S309 VH amino acid | 28 | QVQLVQSGAEVKKPGASVKVSCKASGYPFTSYGISWV |
| sequence | | RQAPGQGLEWMGWISTYNGNTNYAQKFQGRVTMTT |
| | | DTSTTTGYMELRRLRSDDTAVYYCARDYTRGAWFGE |
| | | SLIGGFDNWGQGTLVTVSS |
| S309 VL amino acid | 29 | EIVLTQSPGTLSLSPGERATLSCRASQTVSSTSLAWYQ |
| sequence | | QKPGQAPRLLIYGASSRATGIPDRFSGSGSGTDFTLTIS |
| | | RLEPEDFAVYYCQQHDTSLTFGGGTKVEIK |
| S2X259 VH amino | 30 | QVQLVQSGAEVKKPGSSVKVSCKASGGIFNTYTISWV |
| acid sequence | | RQAPGQGLEWMGRIILMSGMANYAQKIQGRVTITAD |
| | | KSTSTAYMELTSLRSDDTAVYYCARGFNGNYYGWGD |
| | | DDAFDISGQGTLVTVYS |
| S2X259 VL amino | 31 | QTVLTQPPSVSGAPGQRVTISCTGSNSNIGAGYDVHW |
| acid sequence | | YQQLPGTAPKLLICGNSNRPSGVPDRFSGSKSGTSASL |
| | | AITGLQAEDEADYYCQSYDSSLSGPNWVFGGGTKLTV |
| | | L |
| S2X259-v5 VH amino | 32 | QVQLVQSGAEVKKPGSSVKVSCKASGGIFNTYTISWV |
| acid sequence | | RQAPGQGLEWMGRIILMSGMANYAQKIQGRVTITAD |
| | | KSTSTAYMELTSLRSDDTAVYYCARGFNGNYYGWGD |
| | | DDAFDIWGQGTLVTVSS |
| S2X259-variant VL | 33 | QTVLTQPPSVSGAPGQRVTISCTGSNSNIGAGYDVHW |
| amino acid sequence | | YQQLPGTAPKLLIYGNSNRPSGVPDRFSGSKSGTSASL |
| | | AITGLQAEDEADYYCQSYDSSLSGPNWVFGGGTKLTV |
| | | L |
| FM08 VH amino acid | 34 | QVQLQQSGPGLVKPSQTLSLTCAISGDSVSSYNAVWN |
| sequence | | WIRQSPSRGLEWLGRTYYRSGWYNDYAESVKSRITIN |
| | | PDTSKNQFSLQLNSVTPEDTAVYYCARSGHITVFGVN |
| | | VDAFDMWGQGTMVTVSS |
| FM08 VL amino acid | 35 | DIQMTQSPSSLSASVGDRVTITCRTSQSLSSYTHWYQQ |
| sequence | | KPGKAPKLLIYAASSRGSGVPSRFSGSGSGTDFTLTISS |
| | | LQPEDFATYYCQQSRTFGQGTKVEIK |
| IgG1m3 CH1-CH3 | 36 | ASTKGPSVFPLAPSSKSTSGGTAALGCLVKDYFPEPVT |
| | | VSWNSGALTSGVHTFPAVLQSSGLYSLSSVVTVPSSSL |
| | | GTQTYICNVNHKPSNTKVDKRVEPKSCDKTHTCPPCP |
| | | APELLGGPSVFLFPPKPKDTLMISRTPEVTCVVVDVSH |
| | | EDPEVKFNWYVDGVEVHNAKTKPREEQYNSTYRVVS |
| | | VLTVLHQDWLNGKEYKCKVSNKALPAPIEKTISKAKG |
| | | QPREPQVYTLPPSREEMTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| IgG1m17,1 CH1-CH3 | 37 | ASTKGPSVFPLAPSSKSTSGGTAALGCLVKDYFPEPVT |
| | | VSWNSGALTSGVHTFPAVLQSSGLYSLSSVVTVPSSSL |
| | | GTQTYICNVNHKPSNTKVDKKVEPKSCDKTHTCPPCP |
| | | APELLGGPSVFLFPPKPKDTLMISRTPEVTCVVVDVSH |
| | | EDPEVKFNWYVDGVEVHNAKTKPREEQYNSTYRVVS |
| | | VLTVLHQDWLNGKEYKCKVSNKALPAPIEKTISKAKG |
| | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| IgG1m3,1 CH1-CH3 | 38 | ASTKGPSVFPLAPSSKSTSGGTAALGCLVKDYFPEPVT |
| | | VSWNSGALTSGVHTFPAVLQSSGLYSLSSVVTVPSSSL |
| | | GTQTYICNVNHKPSNTKVDKRVEPKSCDKTHTCPPCP |
| | | APELLGGPSVFLFPPKPKDTLMISRTPEVTCVVVDVSH |
| | | EDPEVKFNWYVDGVEVHNAKTKPREEQYNSTYRVVS |
| | | VLTVLHQDWLNGKEYKCKVSNKALPAPIEKTISKAKG |
| | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| HBC34-v40 VH | 39 | ELQLVESGGGWVQPGGSQRLSCAASGRIFRSFYMSWV |
| amino acid sequence | | RQAPGKGLEWVATINQDGSEKLYVDSVKGRFTISRDN |
| | | AKNSLFLQMNNLRVEDTAVYYCAAWSGNSGGMDV |
| | | WGQGTTVSVSS |
| HBC34-v40 VL amino | 40 | SYELTQPPSVSVSPGQTVSIPCSGDKLGNKNVAWFQH |
| acid sequence | | KPGQSPVLVIYQDSYRPSGIPERFSGSNSGNTATLTISG |
| | | TQAMDEAAYFCQTFDSTTVVFGGGTRLTVL |
| S2X259.1-v3.2 VH | 41 | QVQLVQSGAEVKKPGSSVKVSCKASGGIDQTYTISWV |
| amino acid sequence | | RQAPGQGLEWMGRIILISGRADYAQKIQGRVTITADKS |
| | | TSTAYMELTSLRSDDTAVYYCARGFNANYYGWGDD |
| | | DAFDIWGQGTLVTVSS |
| S2X259.1-v3.2 VL | 42 | QTVLTQPPSVSGAPGQRVTISCTGSNSNIGAGYDVHW |
| amino acid sequence | | YQQLPGTAPKLLIVGQSNRPSGVPDRFSGSKSGTSASL |
| | | AITGLQAEDEADYYCQSYDSSGSAPNWVFGGGTKLT |
| | | VL |
| Sotrovimab VH amino | 43 | QVQLVQSGAEVKKPGASVKVSCKASGYPFTSYGISWV |
| acid sequence | | RQAPGQGLEWMGWISTYQGNTNYAQKFQGRVTMTT |
| | | DTSTTTGYMELRRLRSDDTAVYYCARDYTRGAWFGE |
| | | SLIGGFDNWGQGTLVTVSS |
| Sotrovimab VL amino | 44 | EIVLTQSPGTLSLSPGERATLSCRASQTVSSTSLAWYQ |
| acid sequence | | QKPGQAPRLLIYGASSRATGIPDRFSGSGSGTDFTLTIS |
| | | RLEPEDFAVYYCQQHDTSLTFGGGTKVEIK |
| Human IgG1 CH2- | 45 | APELLAGPDVFLFPPKPKDTLMISRTPEVTCVVVDVSE |
| CH3 amino acid | | EDPEVKFNWYVDGVEVHNAKTKPREEQYNSTYRVVS |
| sequence with G236A, | | VLTVLHQDWLNGKEYKCKVSNKALPAPIEKTISKAKG |
| S239D, and H268E | | QPREPQVYTLPPSRDELTKNQVSLTCLVKGFYPSDIAV |
| mutations | | EWESNGQPENNYKTTPPVLDSDGSFFLYSKLTVDKSR |
| | | WQQGNVFSCSVMHEALHNHYTQKSLSLSPGK |
| S2X259-v5 VL amino | 46 | QTVLTQPPSVSGAPGQRVTISCTGSNSNIGAGYDVHW |
| acid sequence | | YQQLPGTAPKLLIVGNSNRPSGVPDRFSGSKSGTSASL |
| | | AITGLQAEDEADYYCQSYDSSLSGPNWVFGGGTKLTV |
| | | L |
| S2X259.1 VH amino | 47 | QVQLVQSGAEVKKPGSSVKVSCKASGGIDNTYTISWV |
| acid sequence | | RQAPGQGLEWMGRIILISGRADYAQKIQGRVTITADKS |
| | | TSTAYMELTSLRSDDTAVYYCARGFNGNYYGWGDD |
| | | DAFDIWGQGTLVTVSS |
| S2X259.1 VL amino | 48 | QTVLTQPPSVSGAPGQRVTISCTGSNSNIGAGYDVHW |
| acid sequence | | YQQLPGTAPKLLIVGNSNRPSGVPDRFSGSKSGTSASL |
| | | AITGLQAEDEADYYCQSYDSSGSAPNWVFGGGTKLT |
| | | VL |

### EXAMPLES

### Example 1

### Increased FcgRIIa Binding Improves Prophylaxis and Enhances Efficacy of Antibodies in a Mouse Model of Influenza a Infection

Anti-influenza A monoclonal antibodies bearing a wild-type Fc domain or a Fc domain comprising a known mutation or mutations were tested in a murine model of infection. Mice expressing human FcγRs were intravenously administered anti-influenza A monoclonal antibodies two days prior to intranasal infection with a lethal dose of H1N1 PR8 (FIG. 1A). Serum IgG levels were evaluated at the time of infection (day 0) and mice were evaluated for body weight and survival over fourteen days.

Mice treated with anti-FluA IgG1 antibody “F18” bearing G236A/A330L/I332E/M428L/N434S Fc mutations retained weight more effectively as compared with mice treated with F18 antibody bearing only M428L/N434S mutations in the Fc (FIG. 1B). Mice treated with antibody bearing G236A (“GA”) or G236A/A330L/I332E (“GAALIE”) Fc mutations maintained weight and had improved survival as compared to mice treated with antibody bearing wild-type Fc or Fc with other modifications (FIGS. 1C and 1D).

### Example 2

### Design of Novel Fc Variants

Human IgG1 Fc regions were engineered for improved function, such as to potentially promote prophylactic, therapeutic, or vaccinal effects by activating certain FcγRs (e.g. FcγRIIA, FcγRIIIA, FcγRIIB). Enhancing activation of FcγRIIA in early infection may promote antibody-dependent cellular phagocytosis (ADCP) and viral neutralization. Enhancing activation of FcγRIIA and/or FcγRIIIA in late or established infection may promote ADCP and/or antibody-dependent cellular cytotoxicity (ADCC), facilitate clearance of virally infected cells, and block viral spread. Enhancing activation of FcγRIIA and/or FcγRIIIA at any time during infection may provide a vaccinal effect by promoting antigen presentation and adaptive immunity.

Fc variants were assessed and new variants were developed using an iterative discovery workflow. An initial set of approximately 2500 Fc point mutations was generated, and functional data was collected and analyzed. Functional data included binding interactions (e.g. to FcγRI, FcγRIIA (R131), FcγRIIB, FcγRIIC, FcγRIIIA (V158), FcRn, and C1q), signaling via FcγRs, thermostability, expressibility, polyreactivity, and half-life extendibility. A machine learning and multi-factor prediction-based algorithm was developed to assist in designing further variants. Fc variants were expressed as anti-influenza A IgG1 antibodies (with FY1 Fabs; Kallewaard et al. Cell 166(3):596-608 (2016)) in CHO cells, titered using high-performance liquid chromatography (HPLC), and purified using protein A columns. A first plate (2×96, with or without 2-deoxy-2-fluoro-L-fucose (2FF), which inhibits fucosylation) contained wells for measuring effects of known mutations (as reference) and wells for measuring effects of novel mutations (single or combination).

Fc variants were analyzed using various assays to evaluate biophysical, biochemical, and biological properties. These included aggregation (e.g. by size-exclusion chromatography), thermostability, glycosylation, structure, signaling, and binding (e.g. using surface plasmon resonance or meso scale discovery-based assays). Effector functions were also tested, including antibody-dependent cellular cytotoxicity (ADCC) and antibody-dependent cellular phagocytosis (ADCP). Binding characteristics of single Fc mutations were evaluated, combinations of up to three mutations were identified that had the highest effect on increasing the IIA/IIB ratio, and additional variations included. The resulting further variants were analyzed. Characteristics of interest included increased affinity for FcγRIIa with reduced affinity for FcγRIIb, or vice versa. Using unbiased cluster analysis and radar plotting with manual analysis, nine clusters of Fc variants having strongly increased, increased, similar or the same, decreased, or strongly decreased affinity for various FcγRs and FcRn were identified.

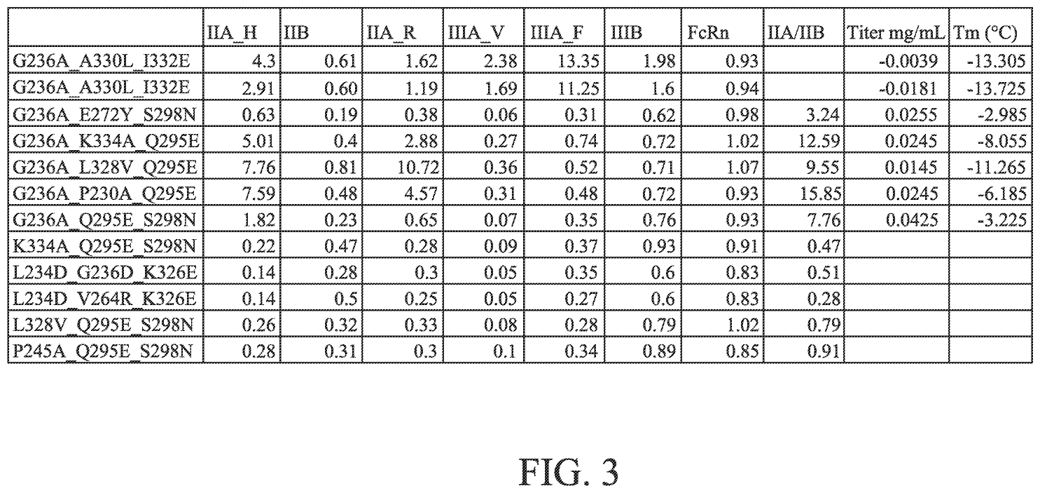

Binding affinities (measure by MSD), Tm, and production titers for certain Fc variants are shown in FIG. 3. Of ten variants that were initially predicted to increase the ratio of FcγRIIA/FcγRIIB binding, five of these (with IIA/IIB values shown in bold in FIG. 3) increased the ratio, as measured by MSD.

### Example 3

### Further Testing of Fc Variants

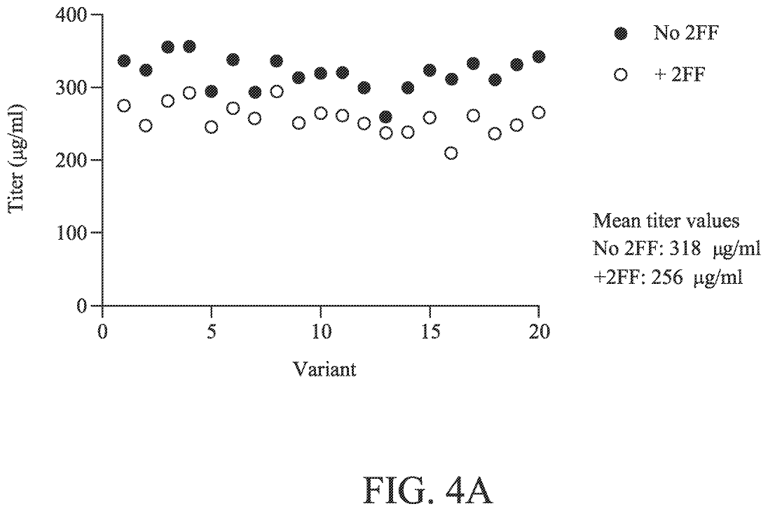

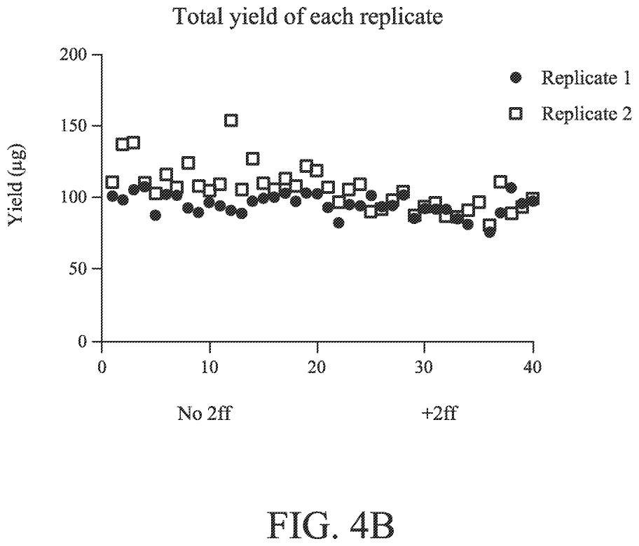

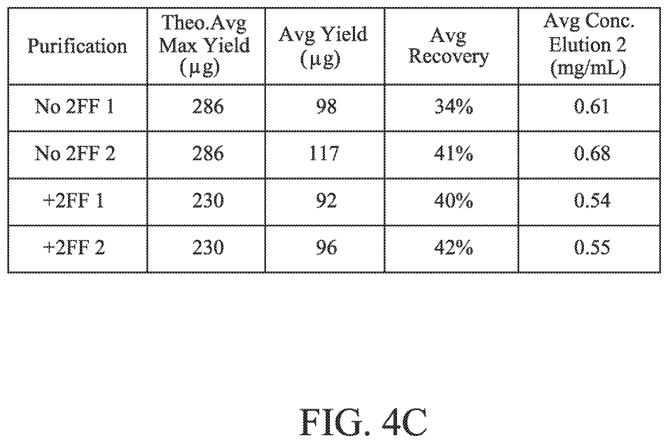

Based on results obtained from the first plate of variants described in Example 2, a second plate (2×20, with and without 2FF) of variants was generated. The twenty Fc variant antibodies were expressed and purified to evaluate titer and yield. Variants were expressed without or with 2FF to determine the effect of fucosylation on titer and yield (FIG. 4A-4C). FIG. 4A shows antibody titers as determined using a Protein A column. Mean titer was higher for variants expressed in cells without 2FF. FIG. 4B shows yields resulting from two replicate purifications (input volume of 900 μL), with two elutions per purification. FIG. 4C summarizes the theoretical maximum yield, average yield, average recovery and protein concentration of the second elution (measured in μg/ml). Fc variants were purified using two elutions and combined prior to determining yield. Average yield was higher in purified Fc variants expressed without 2FF.

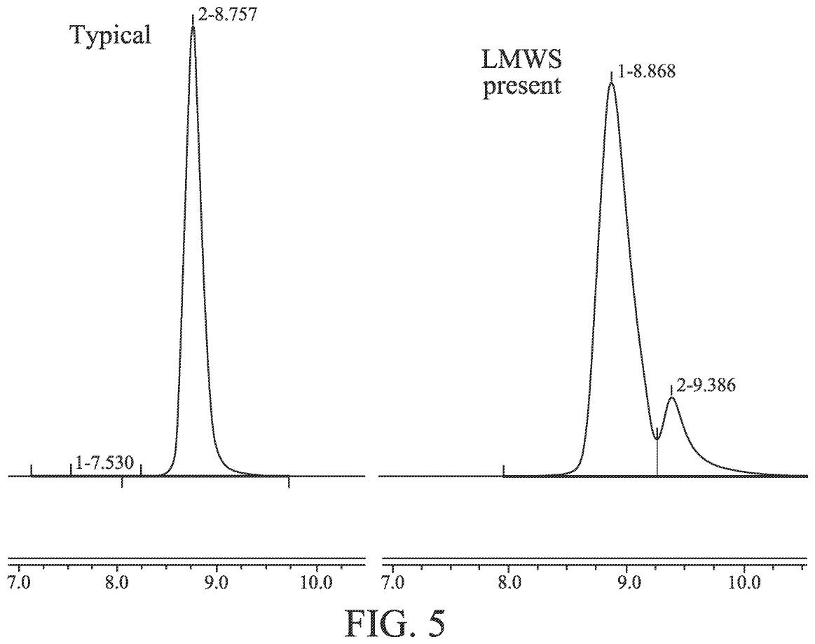

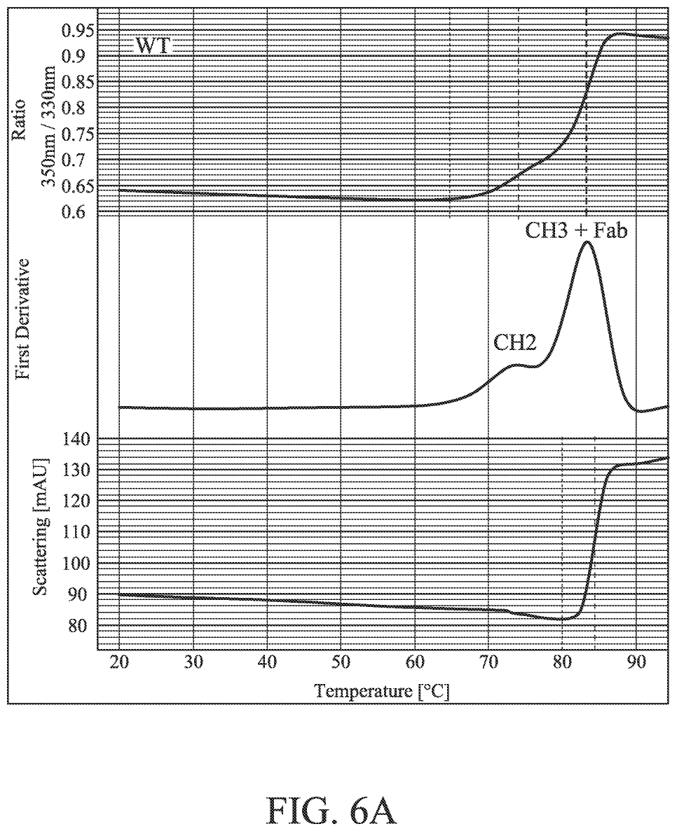

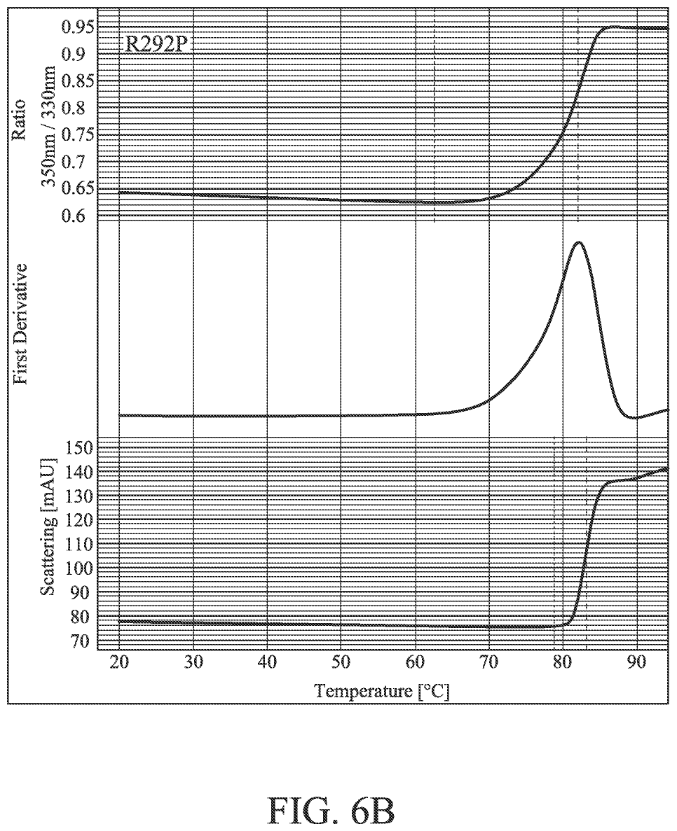

Purified antibodies (+2FF and no 2FF) were analyzed for potential to dimerize using size-exclusion chromatography (FIG. 5) and Tm were evaluated (FIGS. 6A-6B). Low molecular weight species were observed for C220P and R292P_Y300L variants (+2FF samples only). Tm values for certain Fc variants were within 4.2° C. of WT; by comparison, Tm of the GAALIE Fc variant was approximately 14° C. lower relative to WT (tested on two plates). CH2 unfolding, represented by the absence of a second peak in the lower graph of the R292P sample in FIG. 6B, could not be resolved for variants including R292P except for the combination mutant G236A_R292P_I377N.

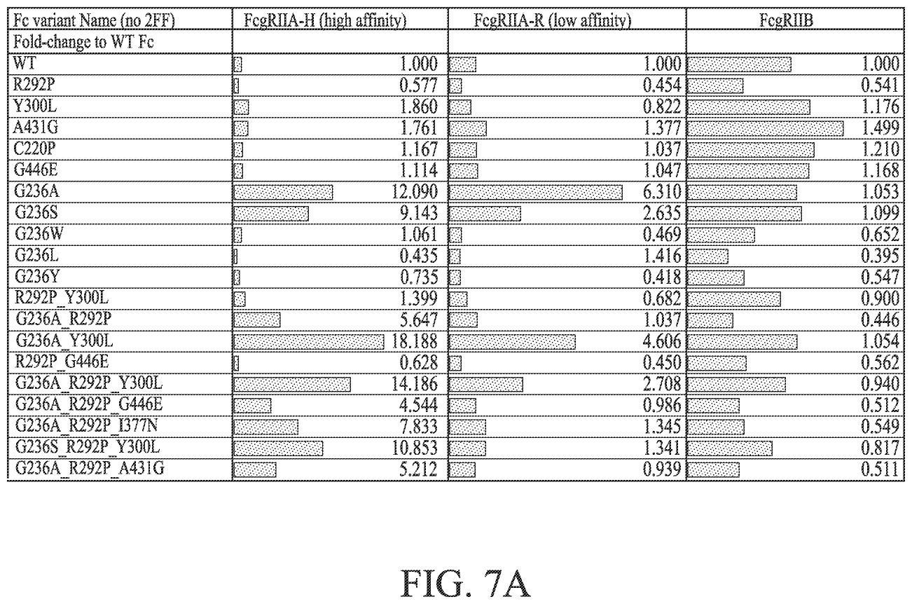

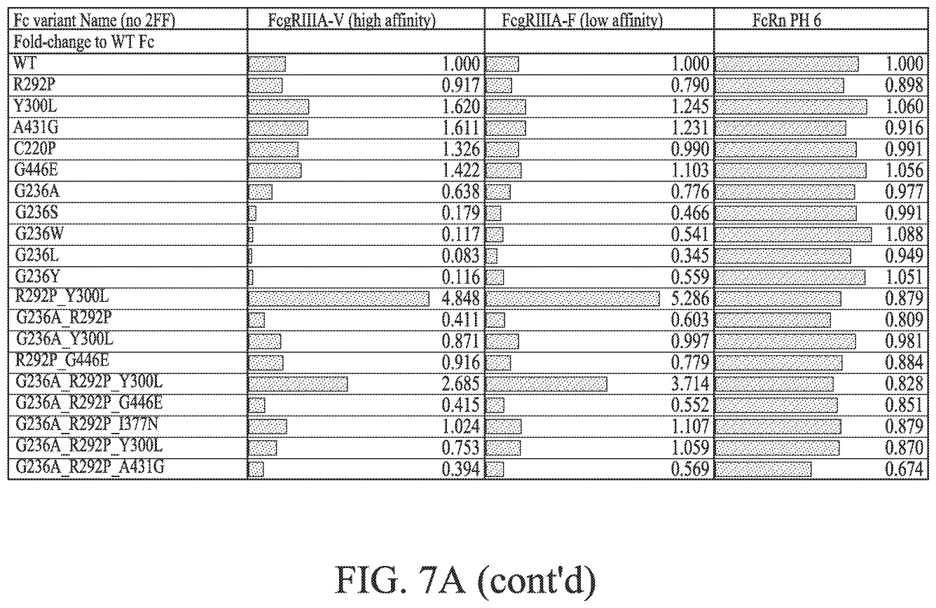

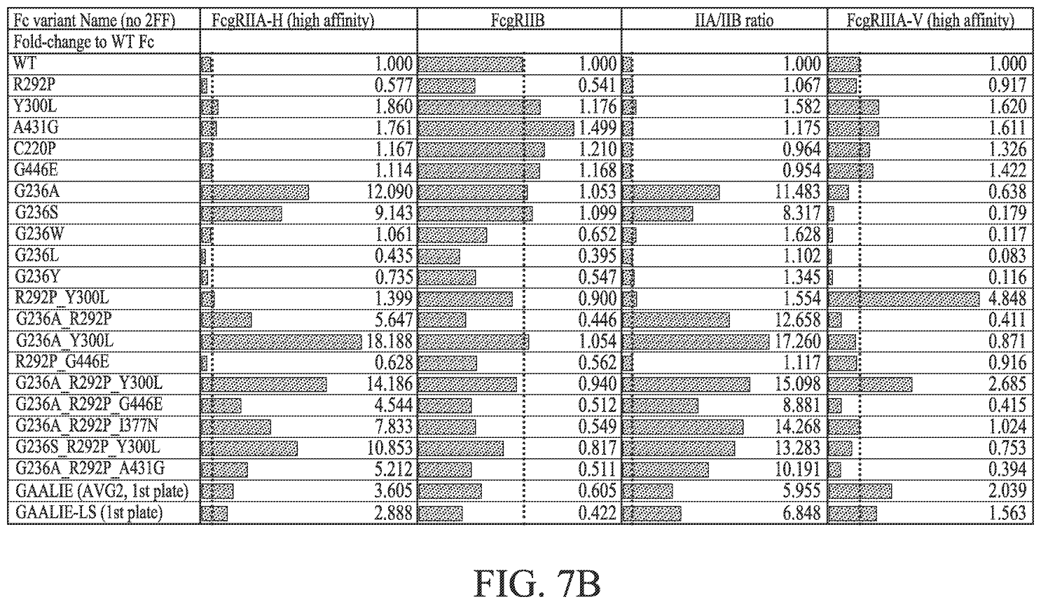

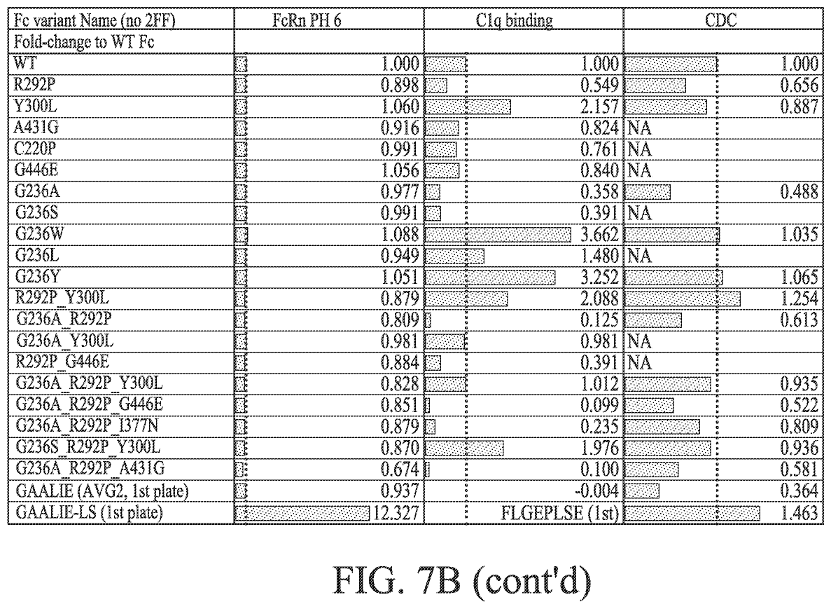

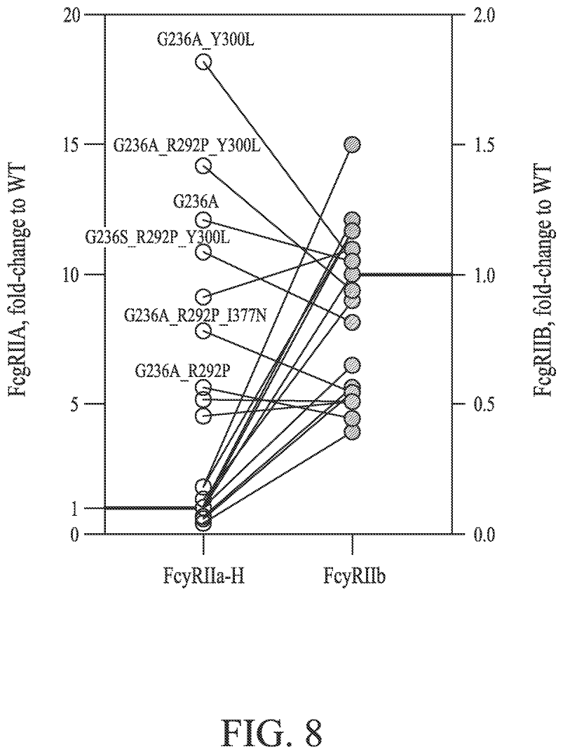

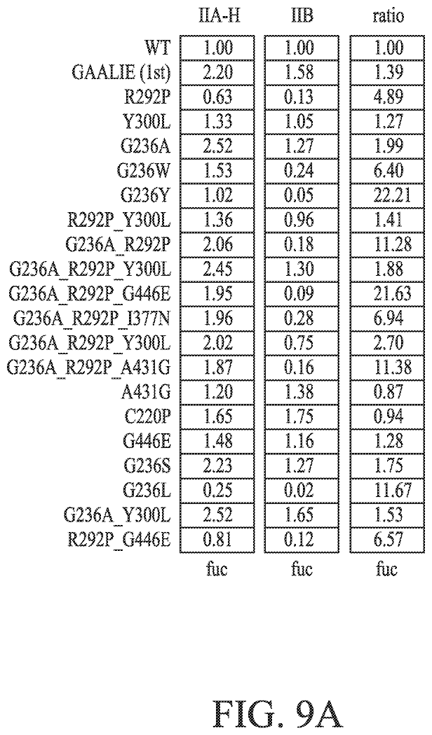

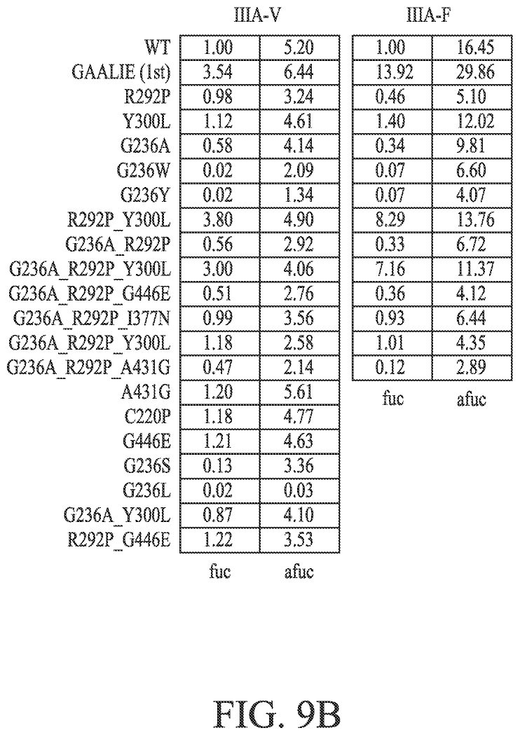

Binding of (No 2FF) Fc variants to FcγRIIA-H (high affinity), FcγRIIA-R (low affinity), FcγRIIB, FcγRIIIA-V (high affinity), FcγRIIIA-F (low affinity), and FcRn (at pH 6) was tested and expressed as fold-change relative to wild-type Fc (FIG. 7A). The ratio of FcγRIIA-H/FcγRIIB binding, as well C1q binding and complement-dependent cytotoxicity (CDC) data, are also shown in FIG. 7B. Variants shown in FIG. 8 were not treated with 2FF. Antibody signaling through different FcγRs was measured using a reporter assay (Promega™ luciferase reporter cells; average of 3 experiments). Fucosylated Fc variants were tested for signalling through all four FcγR receptors shown (FIG. 9A), while afucosylated variants were tested for signalling through FcγRIIIA-V and FcγRIIIA-F (FIG. 9B). A number of variant Fcs were selected for further characterization.

A summary of characteristics of these variants (both fucosylated and afucosylated), as well as of comparator variant Fcs comprising known mutations (e.g., G236A_S239D_A330L_I332E (“GASDALIE”); G236A_A330L_I332E (“GAALIE”) is shown in FIGS. 10A-10C.

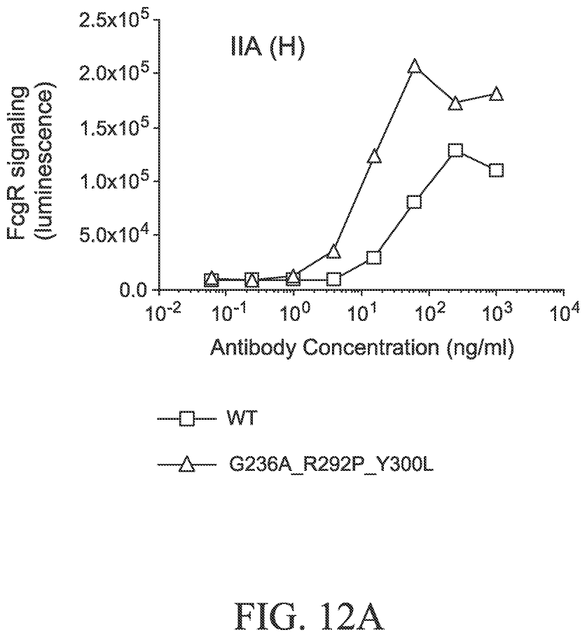

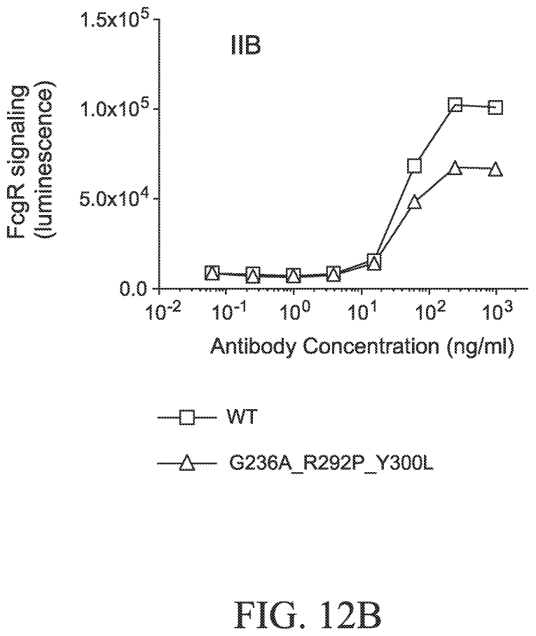

Several variants demonstrated improved attributes over the GAALIE variant, including increased IIA/IIB ratio, increased stability (Tm), balanced binding to FcγR alleles, and increased C1q binding and complement activation. Dose-dependent FcγR signaling through FcγRIIA-H (high affinity, FIG. 12A) and FcγRIIB (FIG. 12B) for one such Fc variant, “G236A_R292P_Y300L” was measured using a reporter cell assay.

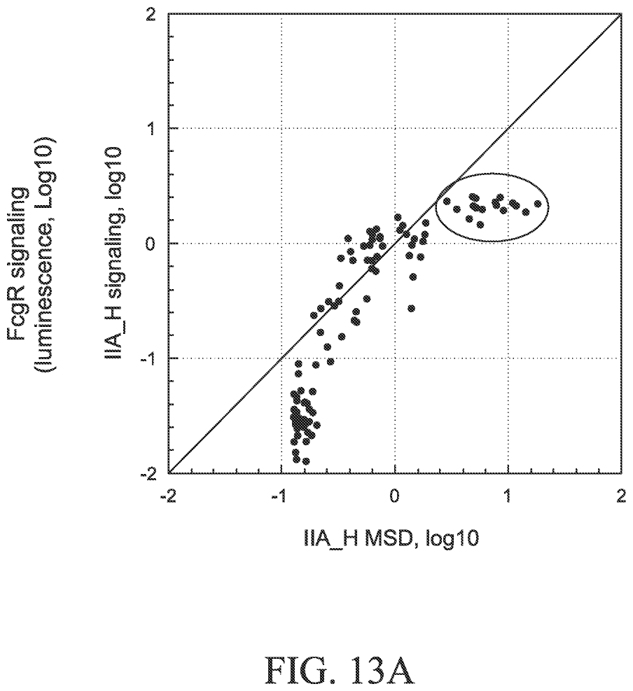

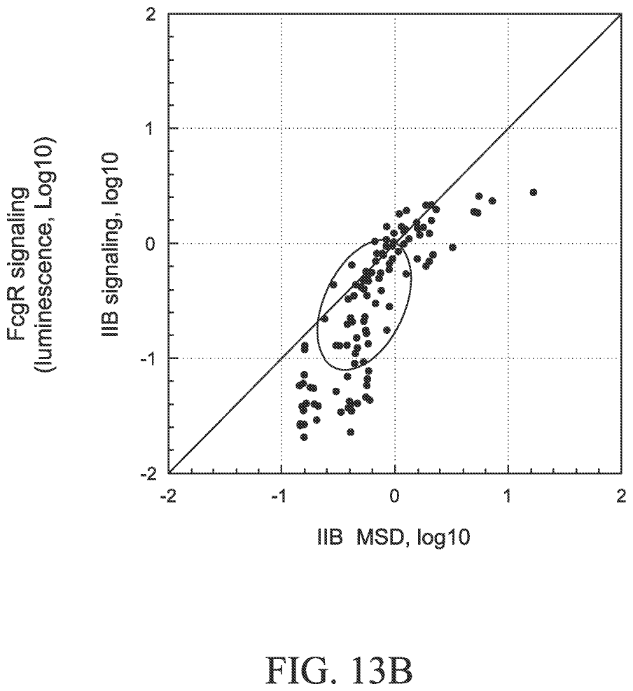

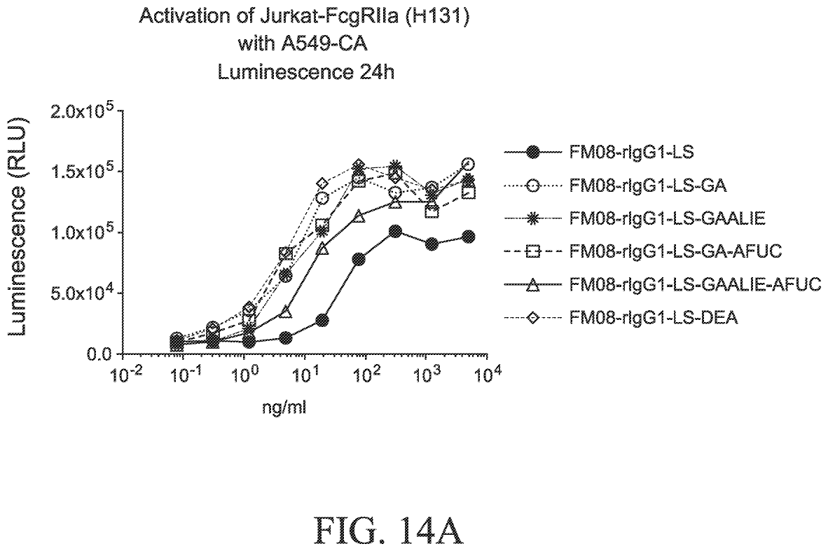

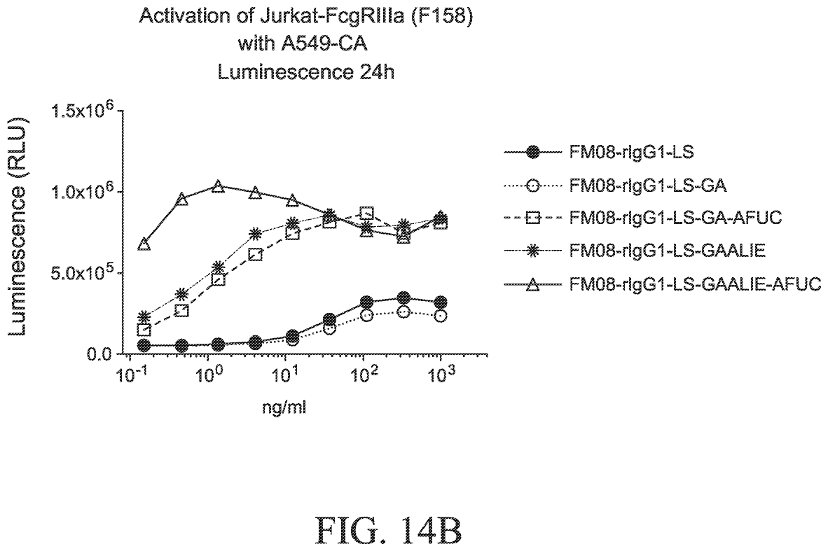

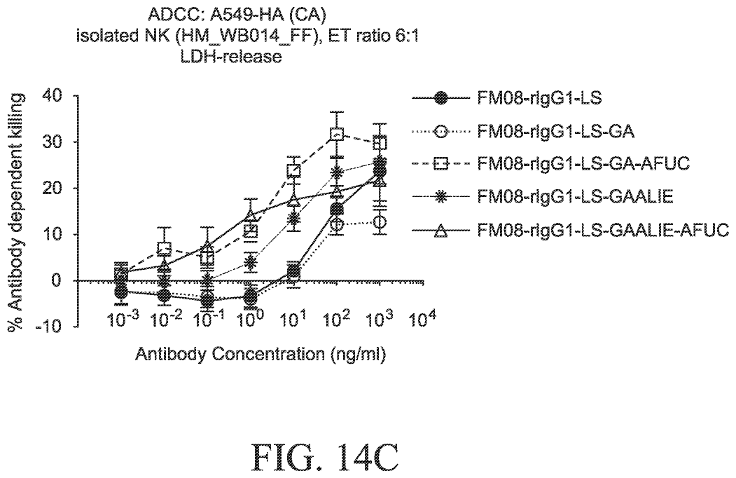

FcγR binding versus signaling through FcγRIIA-H (high affinity, FIG. 13A) and FcγRIIB (FIG. 13B) by Fc variants is shown in FIGS. 13A and 13B. FcγR binding was measured using a Meso Scale Discovery binding assay and FcγR signaling was measured using a reporter cell assay.

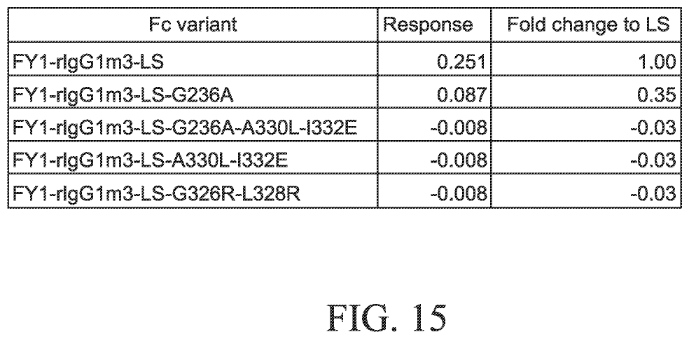

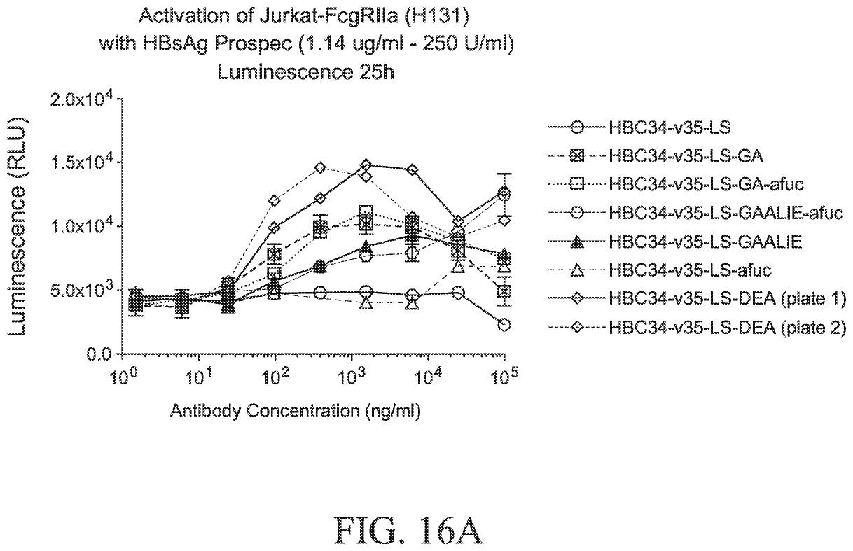

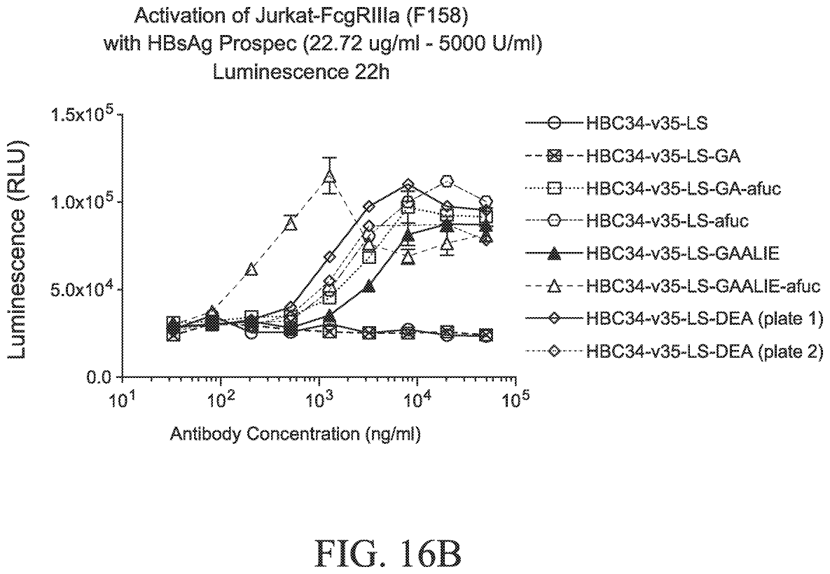

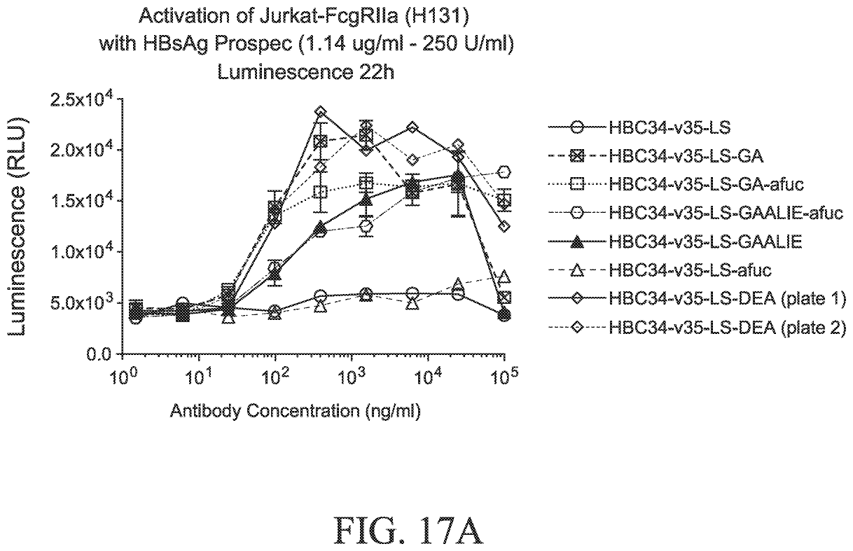

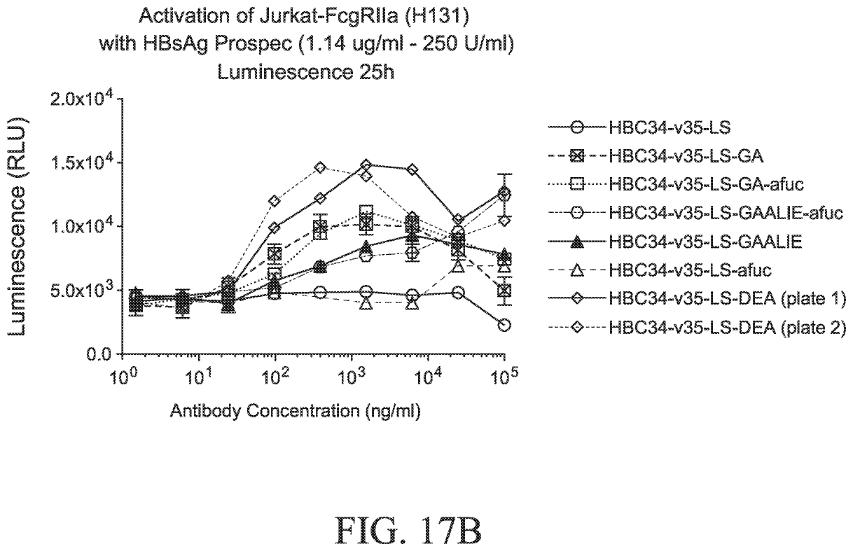

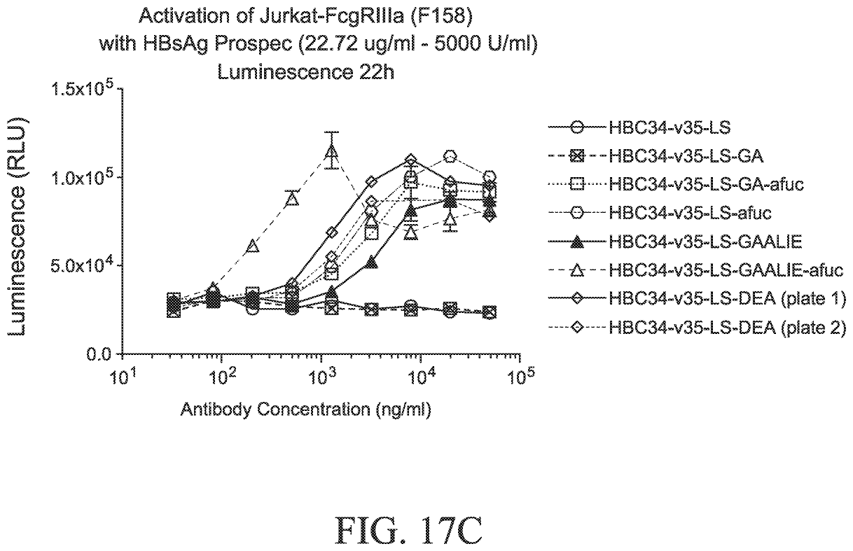

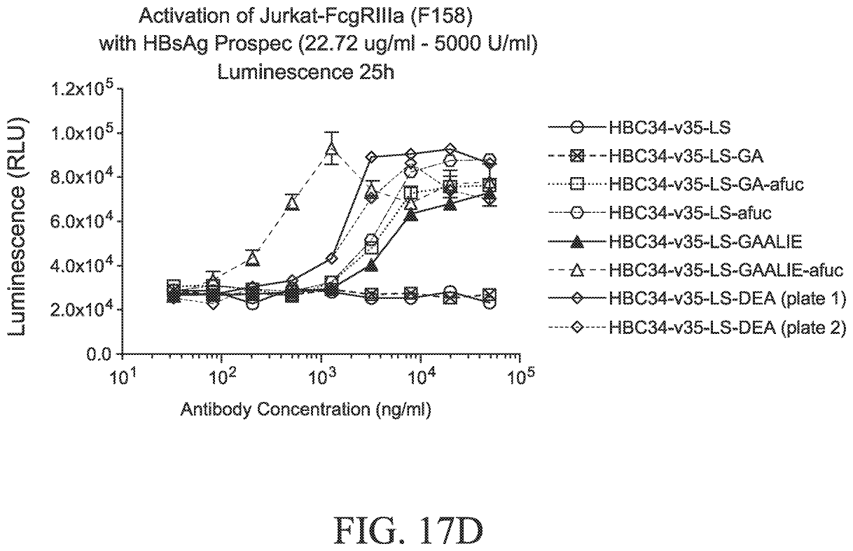

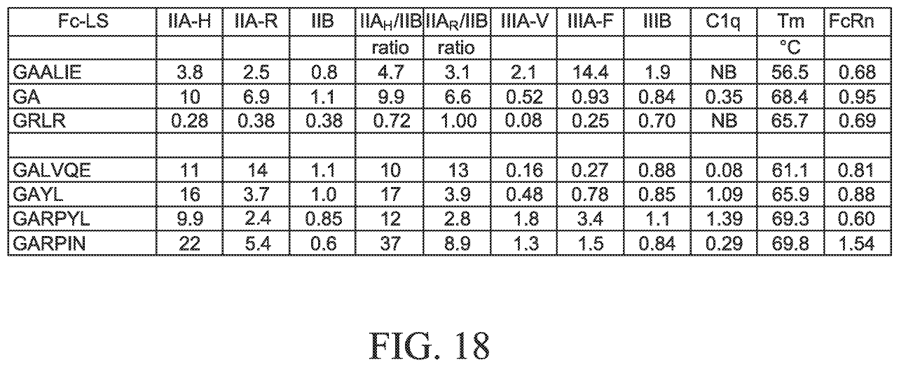

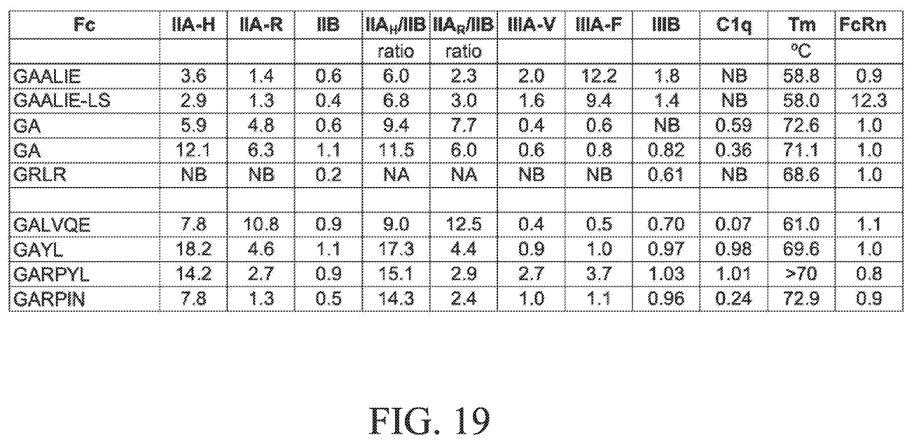

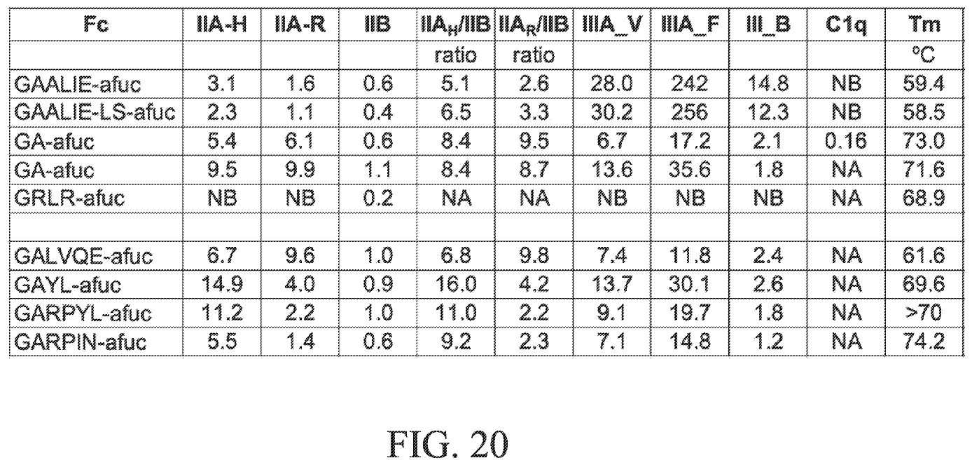

Additional experiments were conducted using two anti-flu HA antibodies (FY1 and FM08) and an anti-HBsAg antibody (HBC34-v35), with results shown/summarized in FIGS. 15-17D. These results showed, inter alia, that afucosylated Fc variant antibodies bearing the G236A (“GA”) mutation have stronger binding to FcγRs IIA and IIIA as compared to fucosylated Fc variant antibodies bearing G236A_A330L_I332E (“GAALIE”), and induce comparable signalling via FcγRIIIA, and potentially stronger signalling via FcγRIIA, as compared to fucosylated GAALIE-bearing Fc variant antibodies. Additionally, GA-afucosylated Fc variant antibodies had improved melting temperature versus fucosylated GAALIE-bearing Fc variant antibodies. GA-afucosylated Fc variant antibodies induced NK cell-mediated ADCC against target cells. GA-afucosylated Fc variant antibodies retained partial C1q binding (0.3× compared to reference IgG1 antibody bearing M428L and N434S mutations only), while GAALIE mutations caused abrogation of C1q binding.

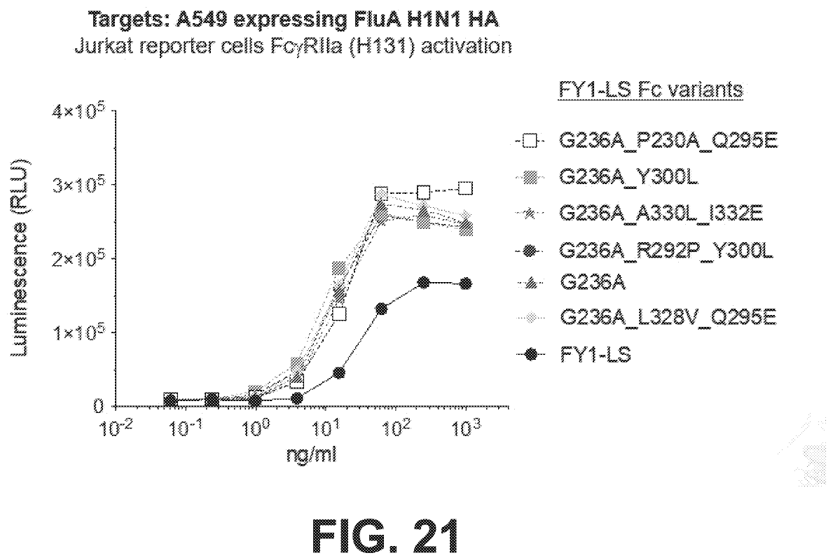

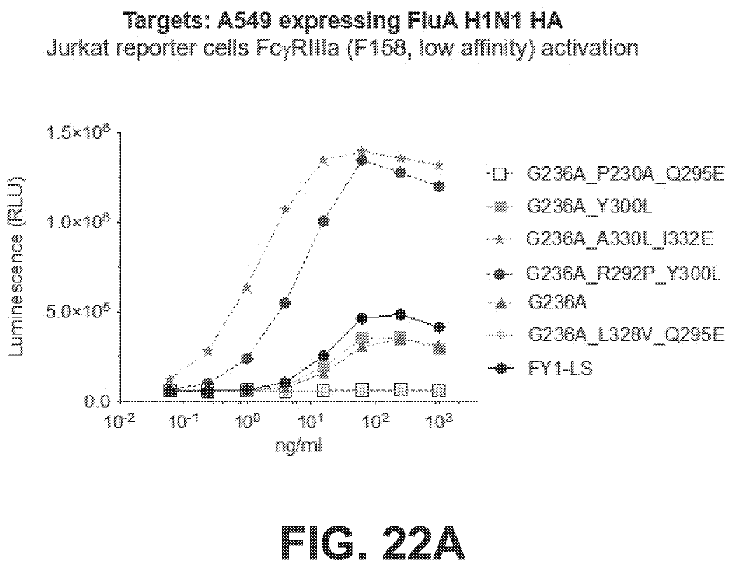

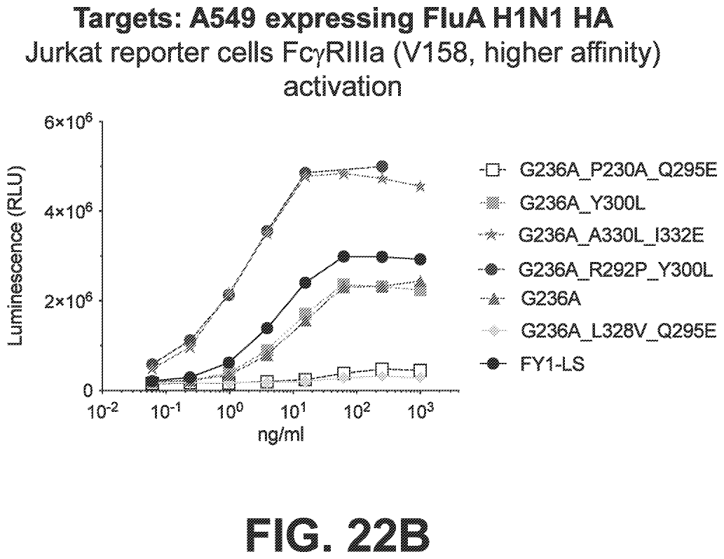

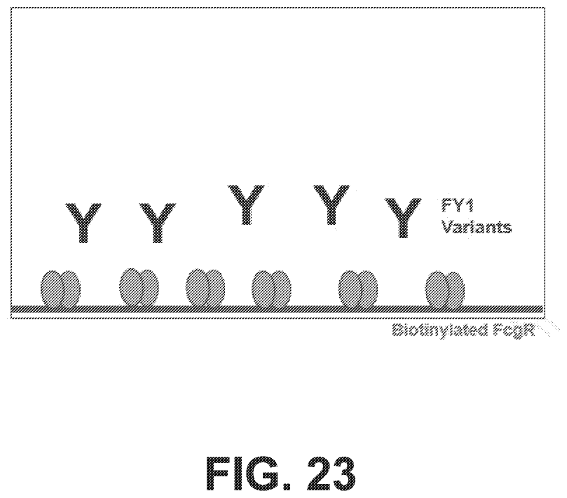

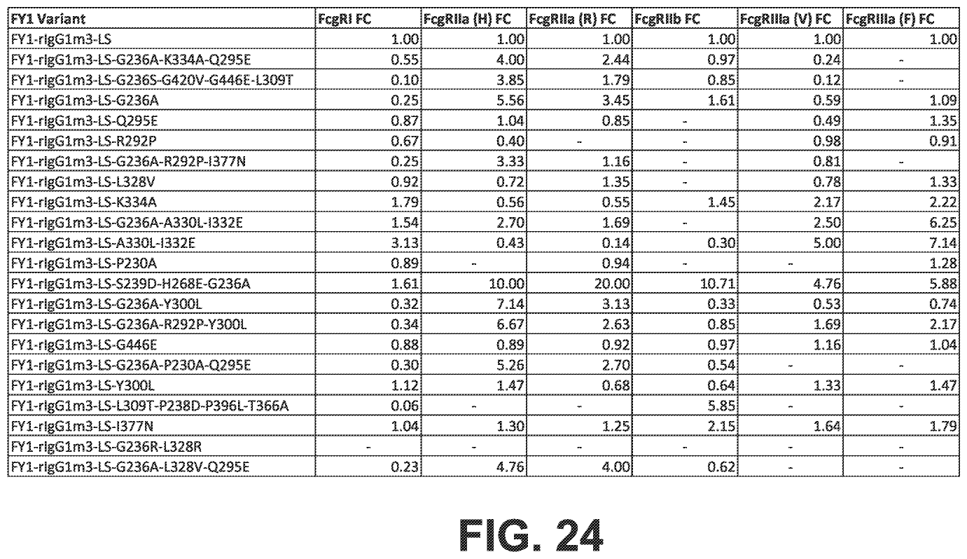

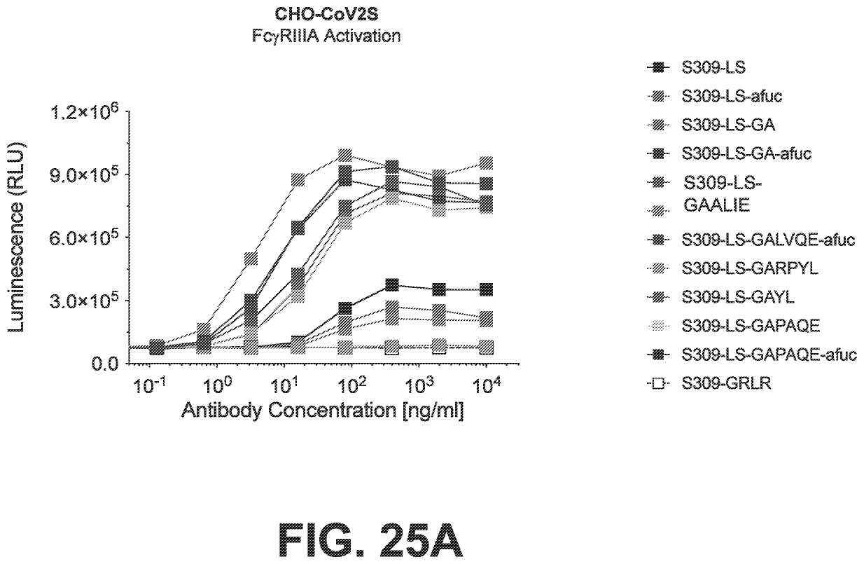

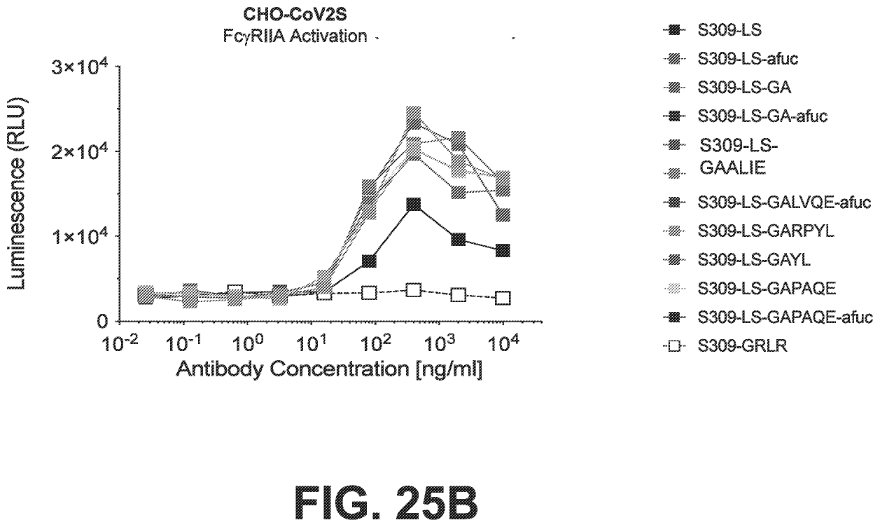

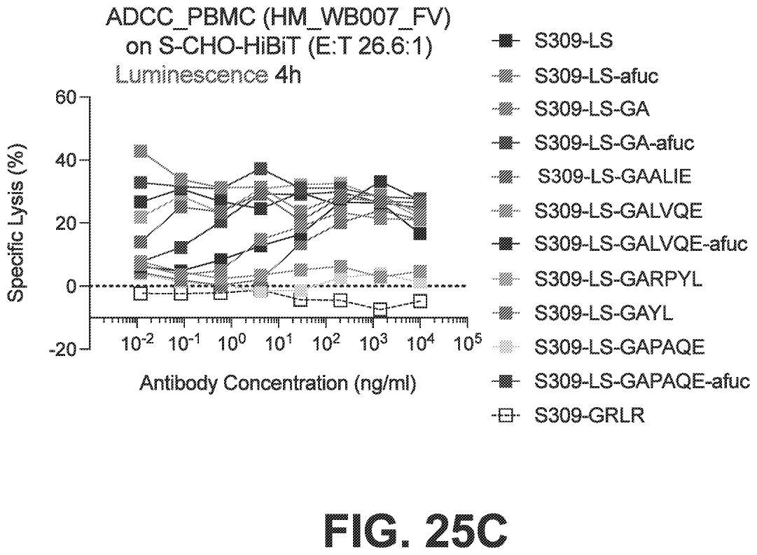

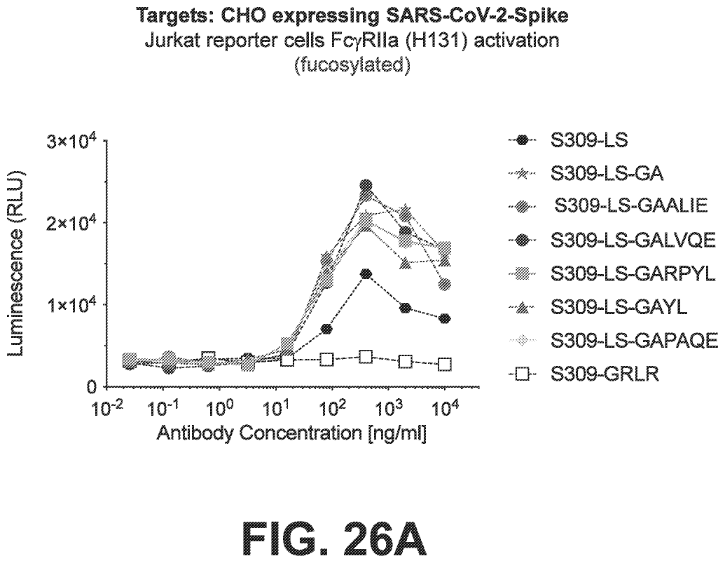

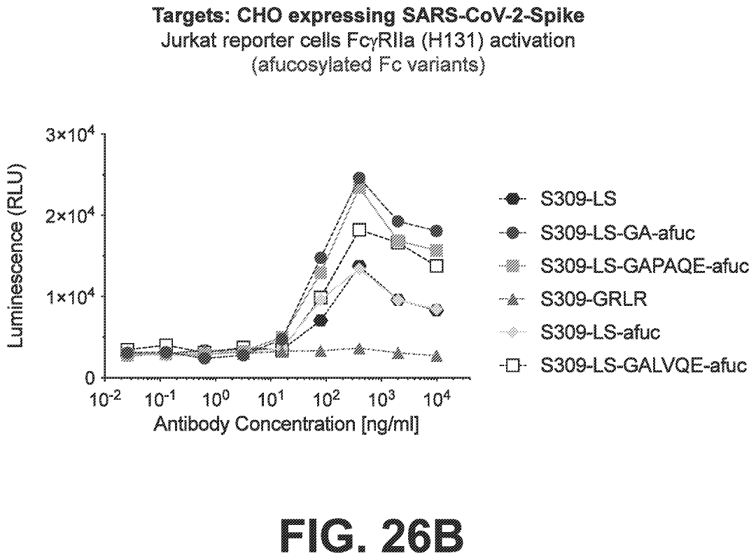

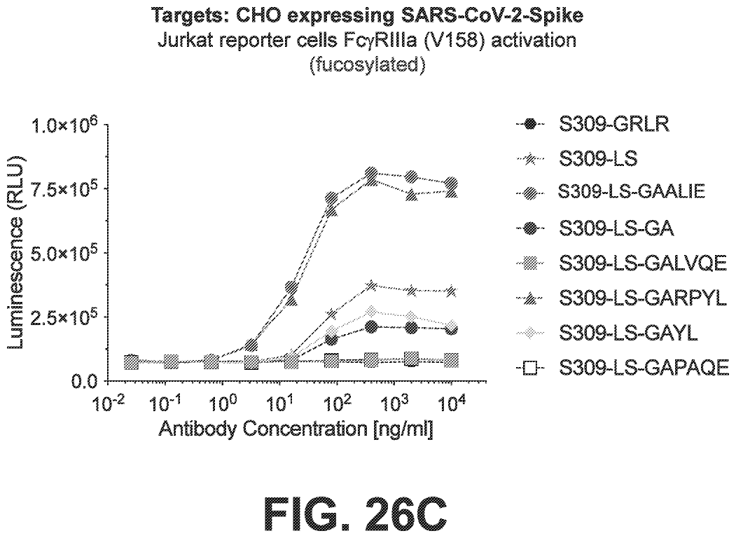

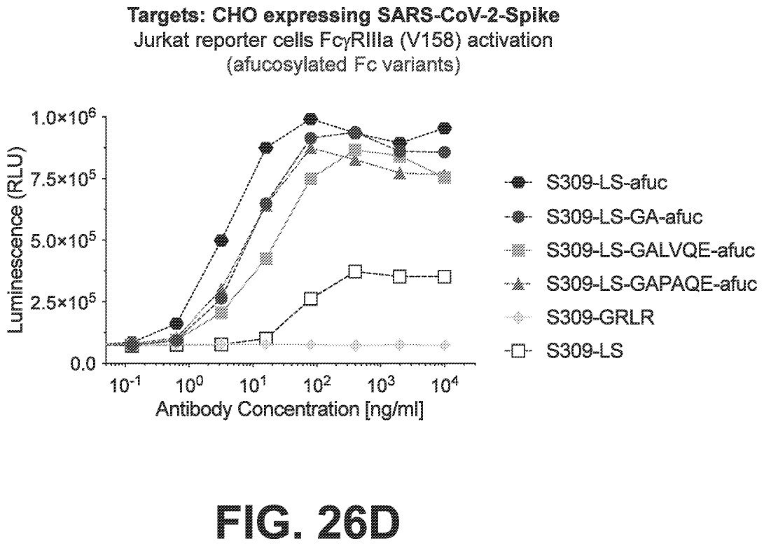

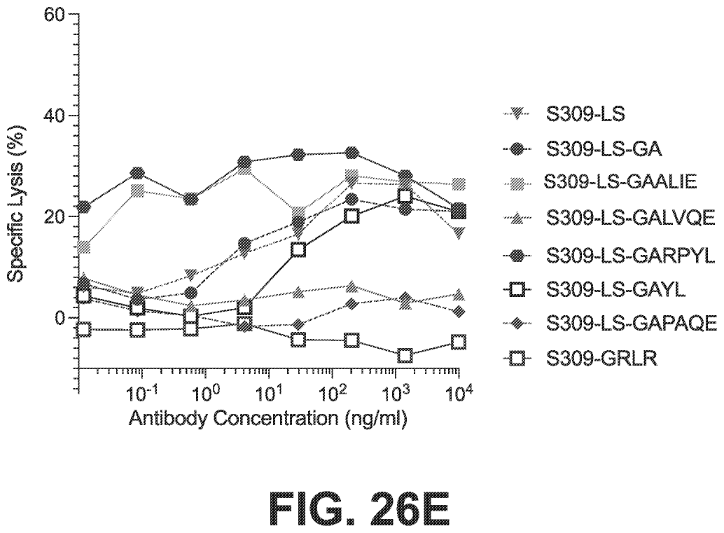

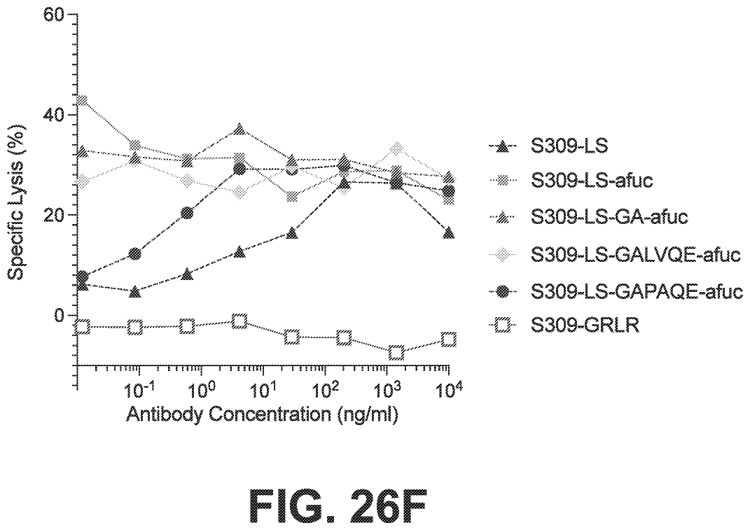

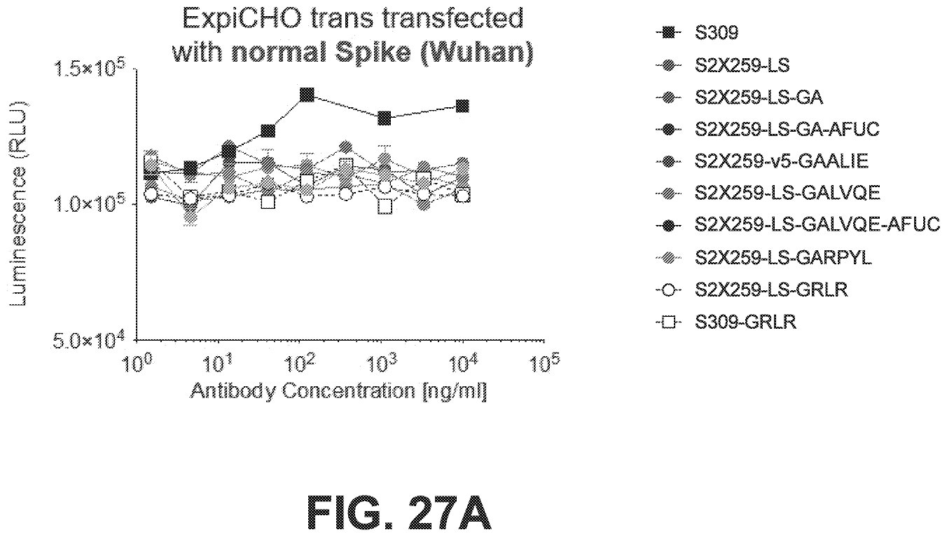

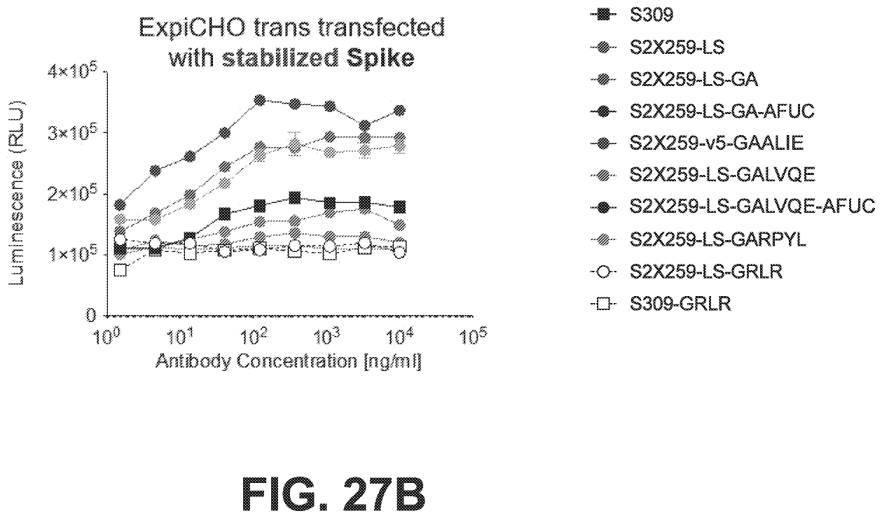

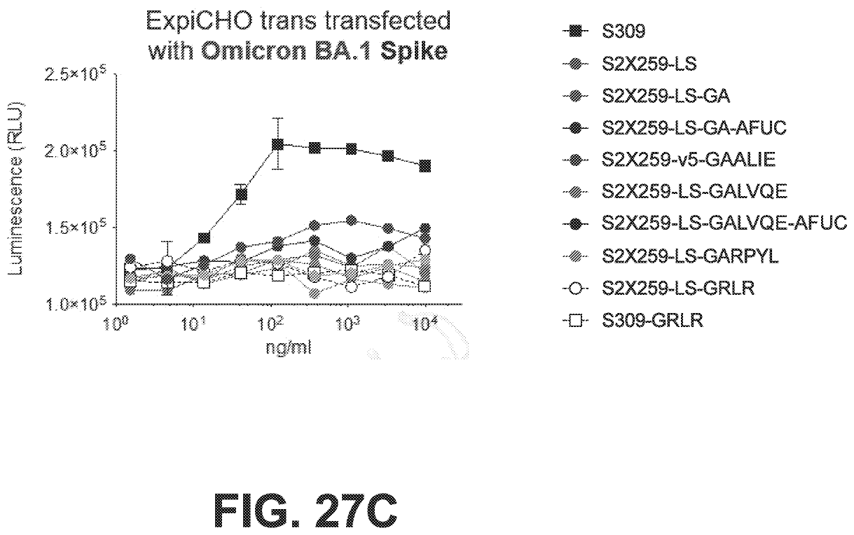

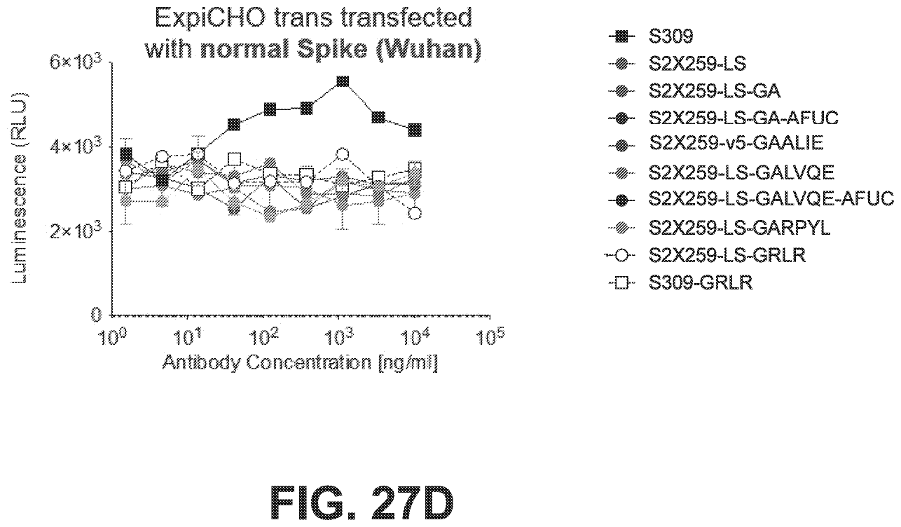

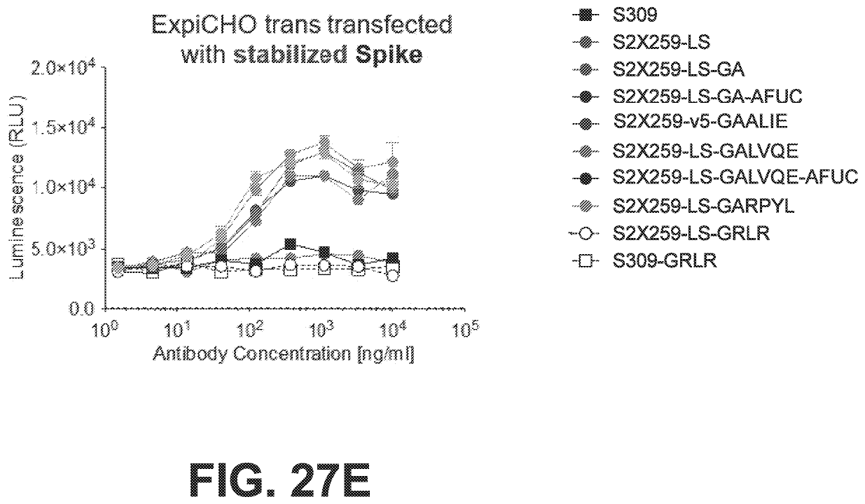

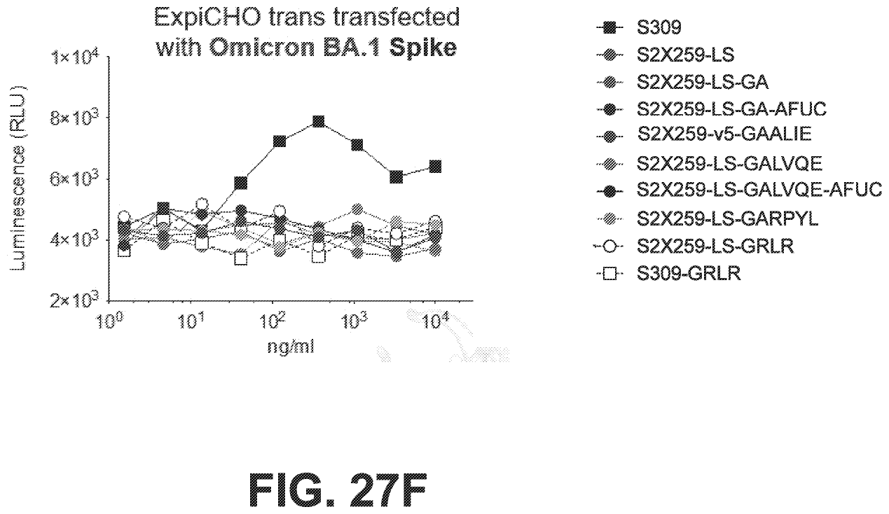

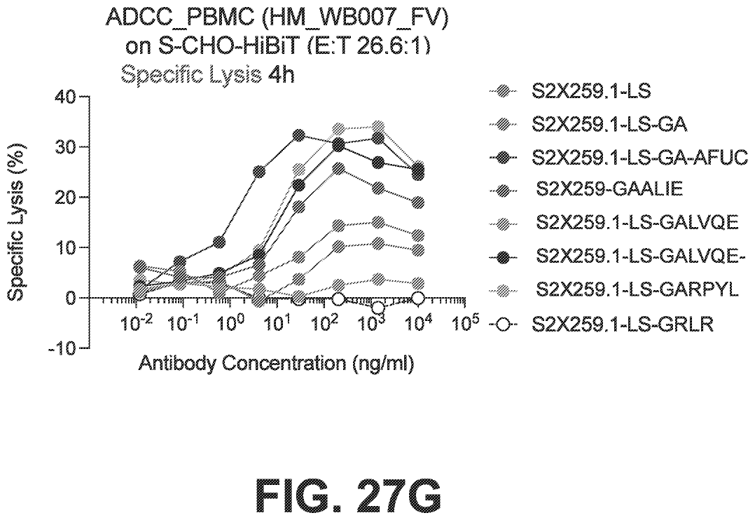

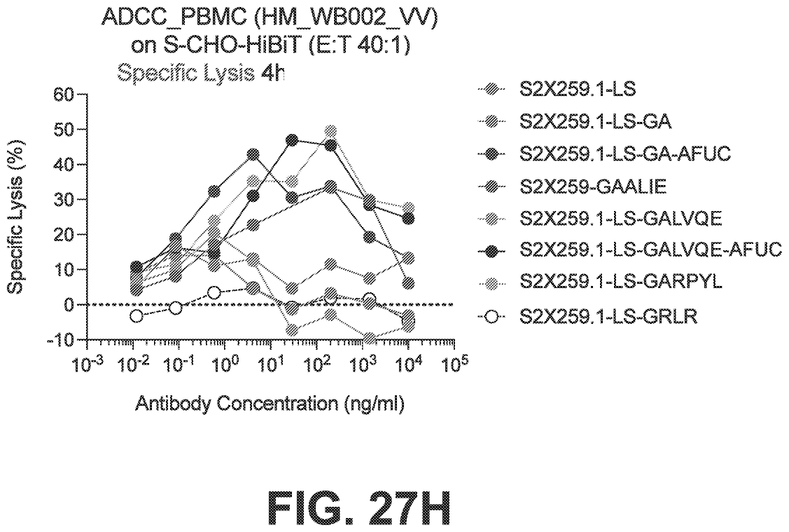

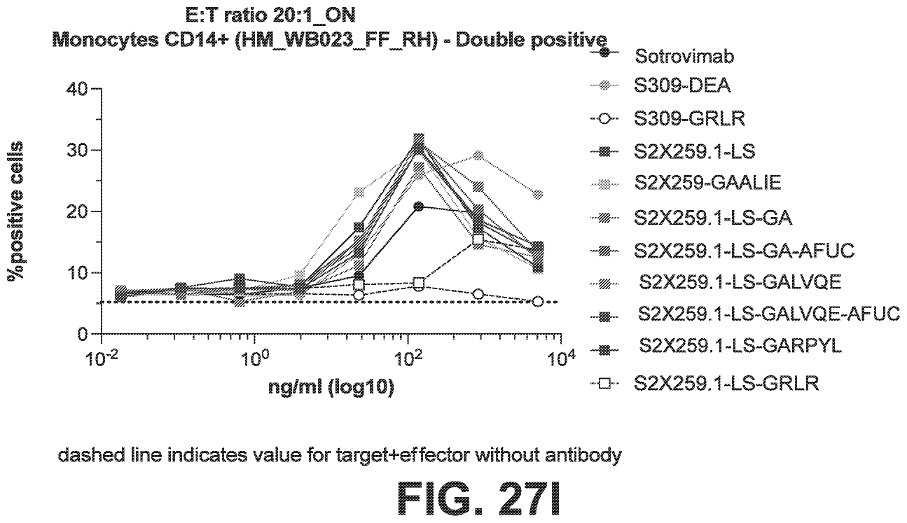

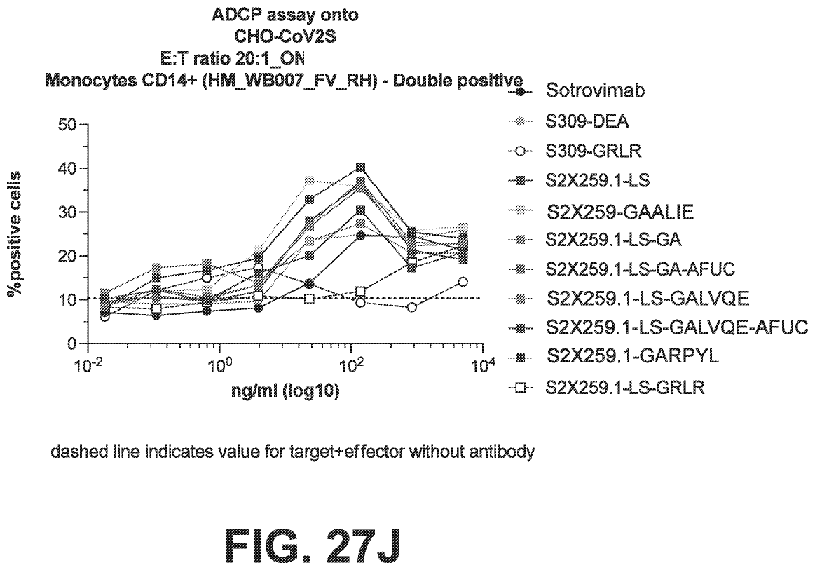

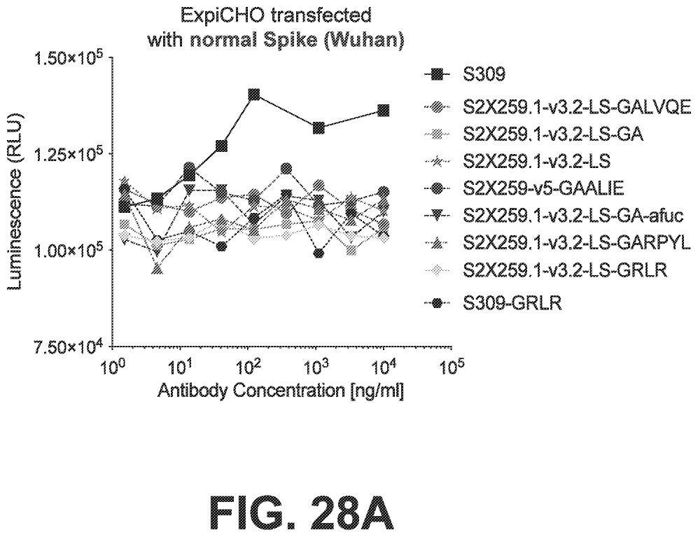

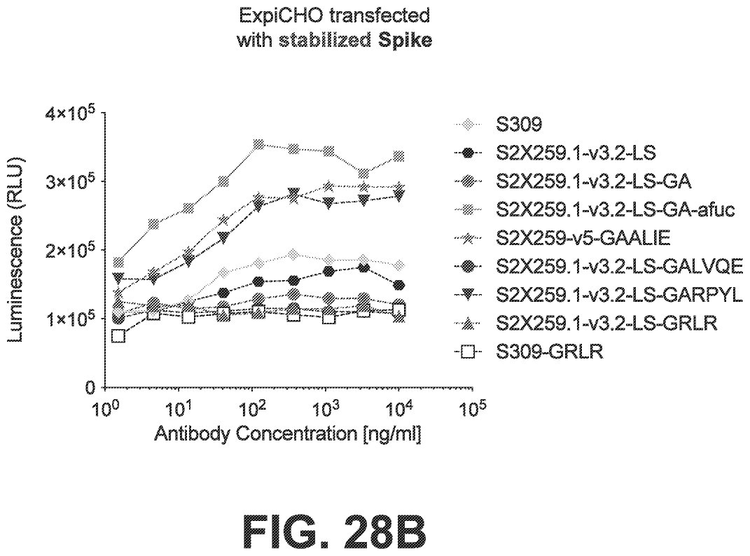

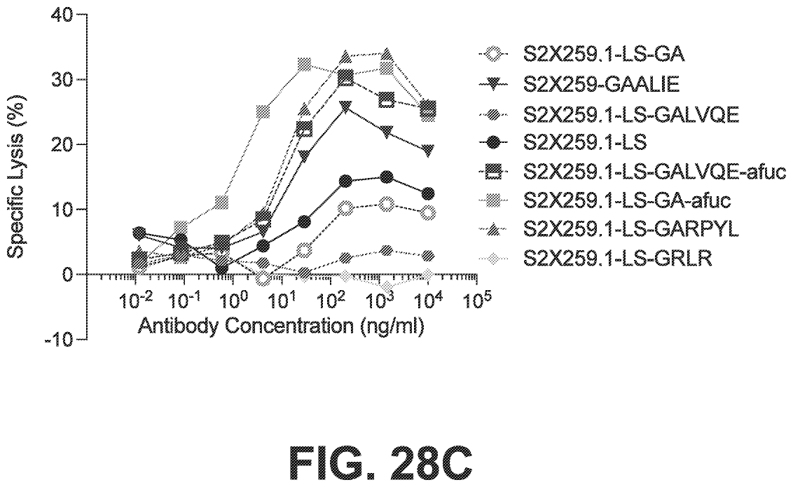

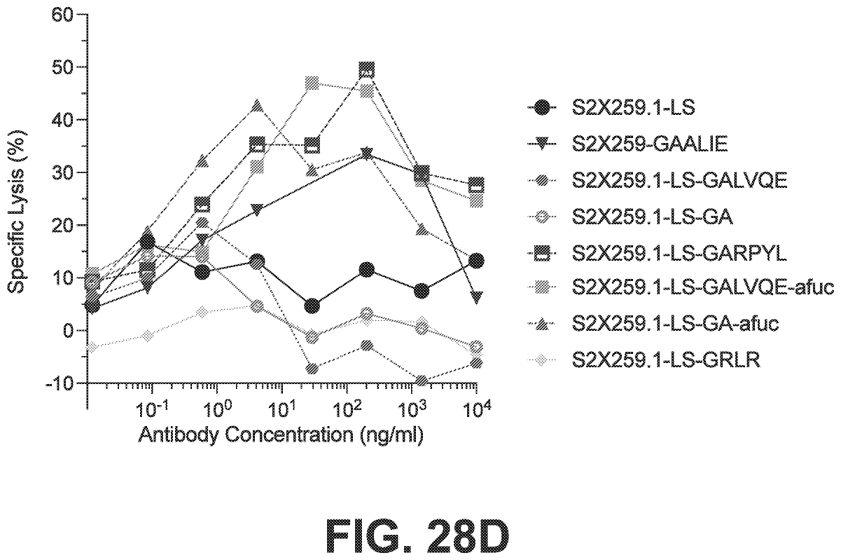

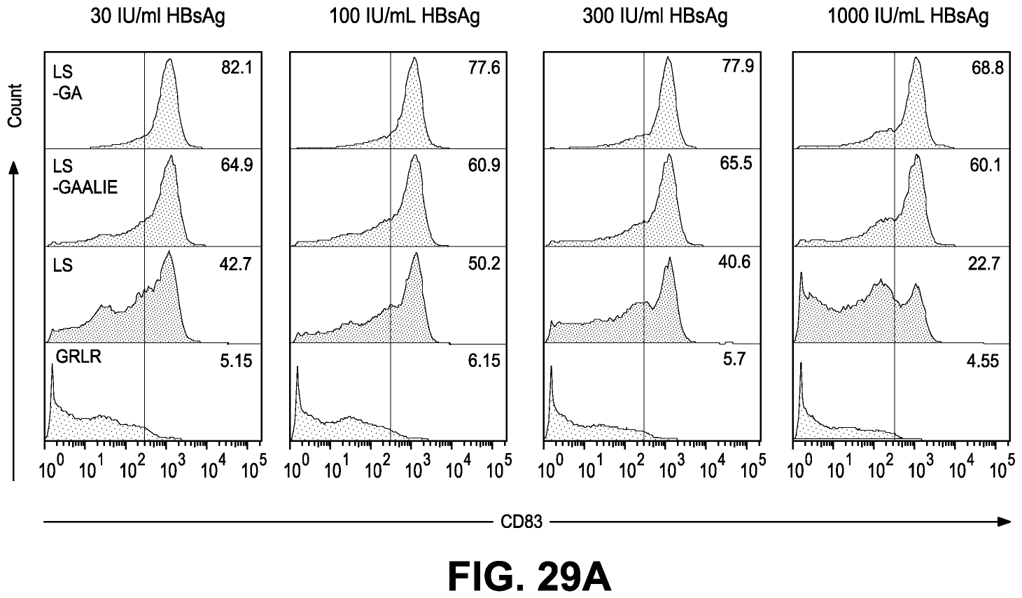

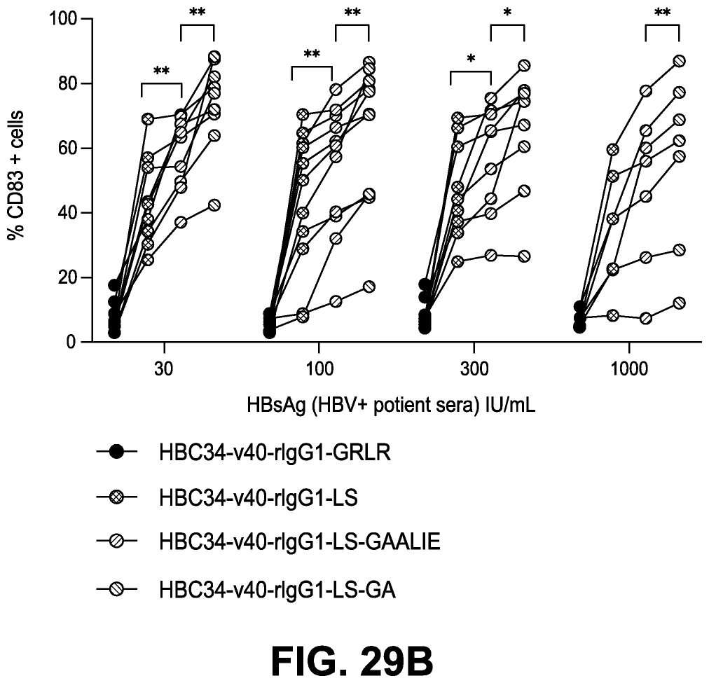

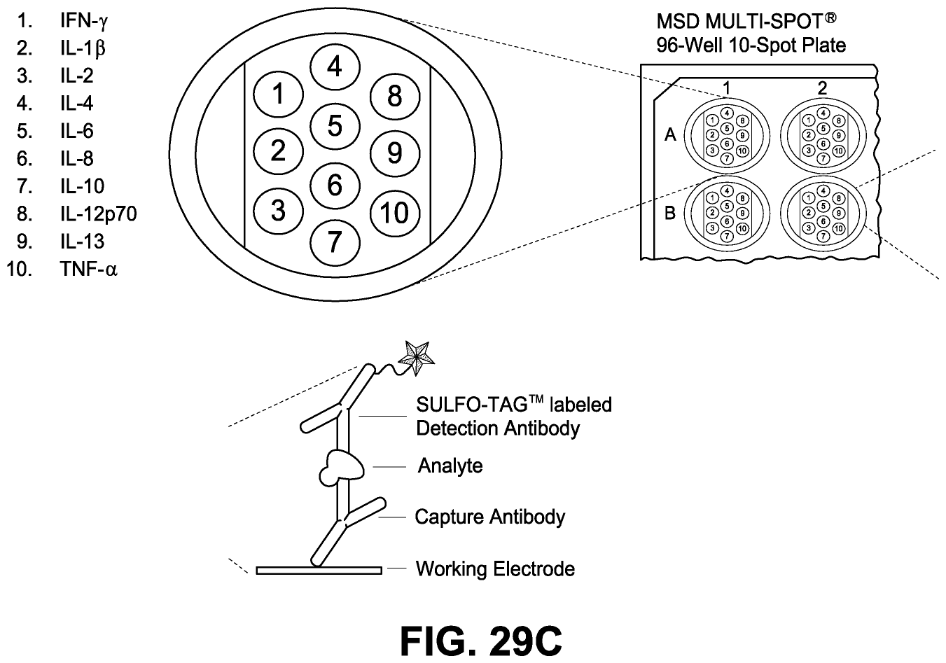

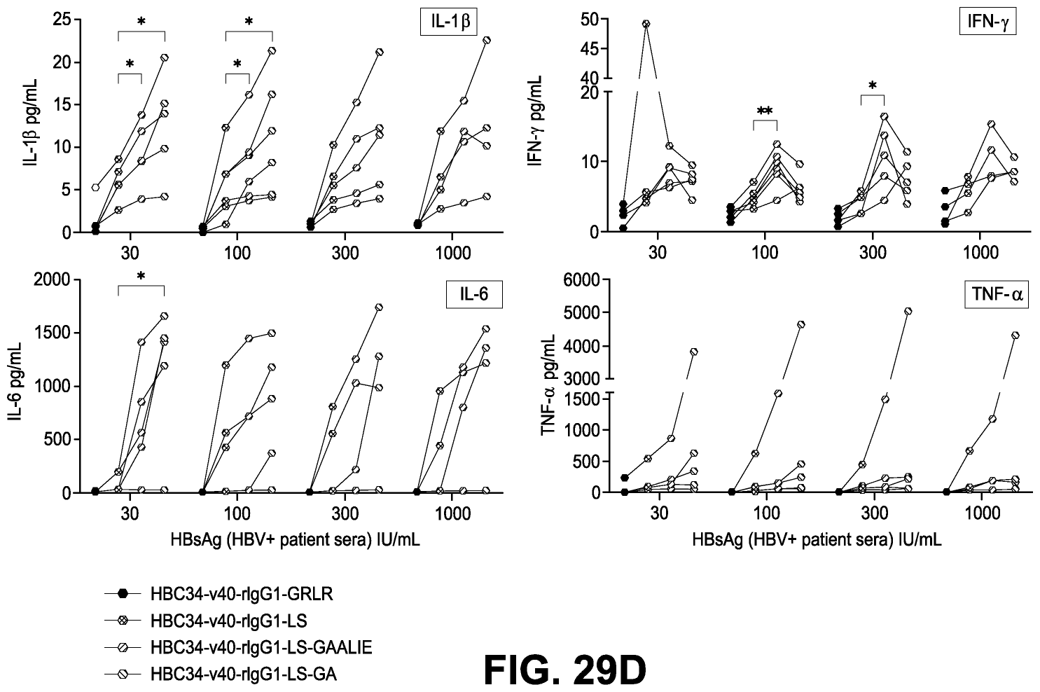

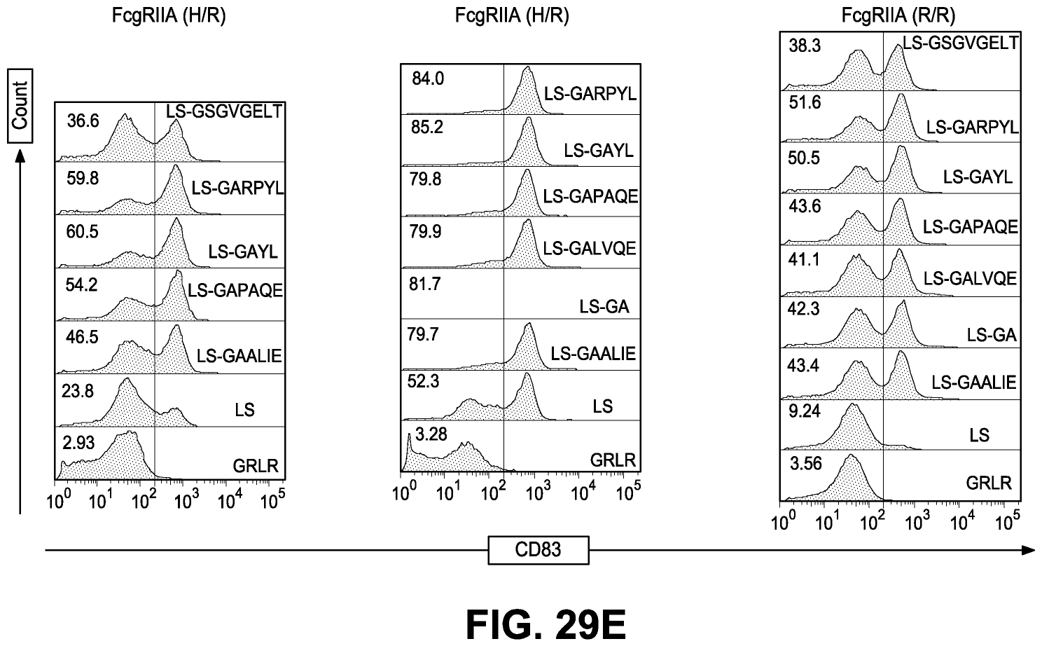

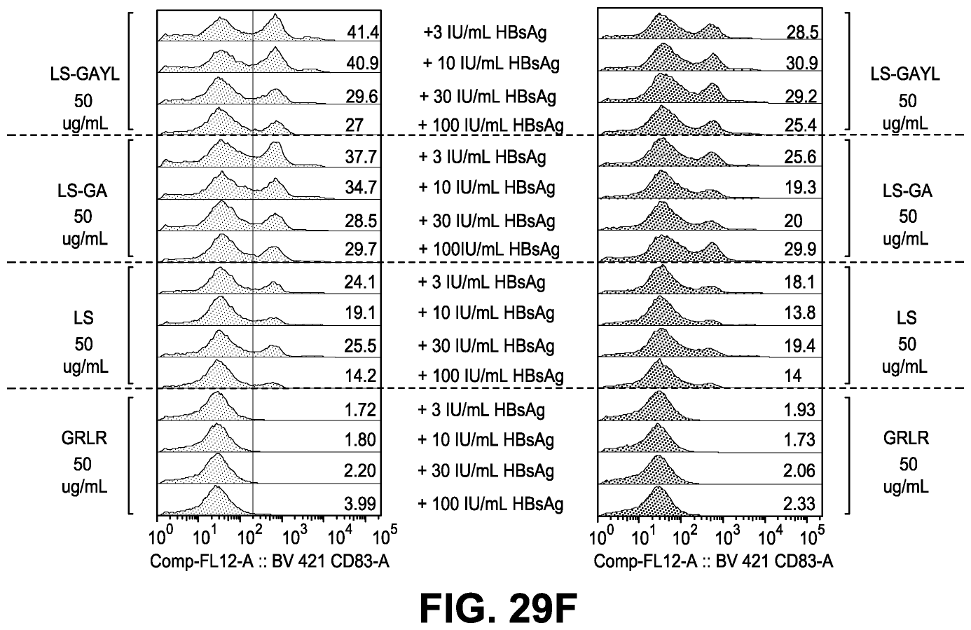

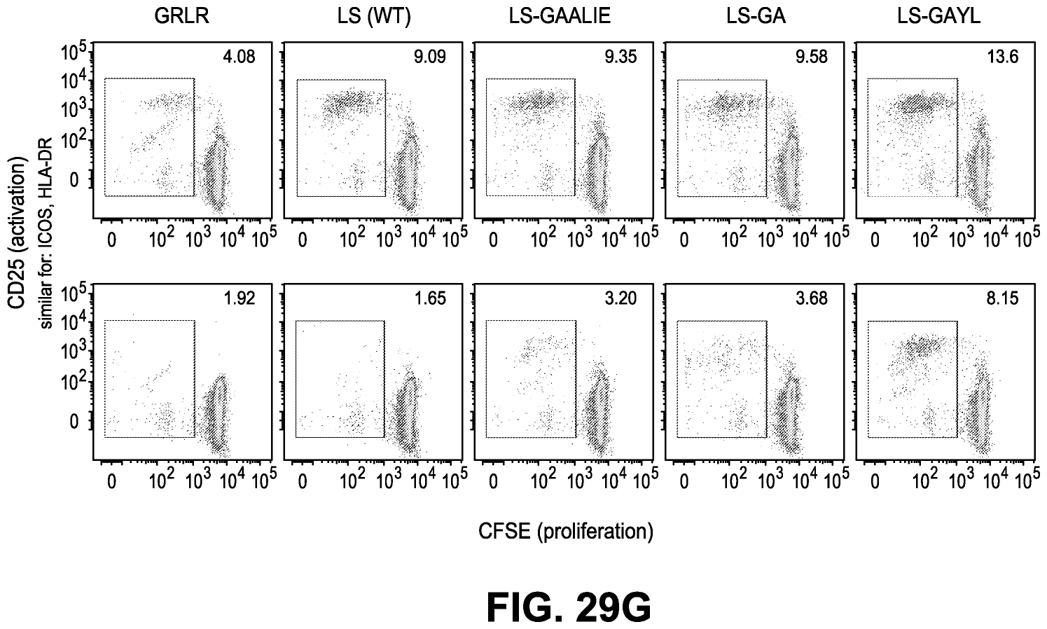

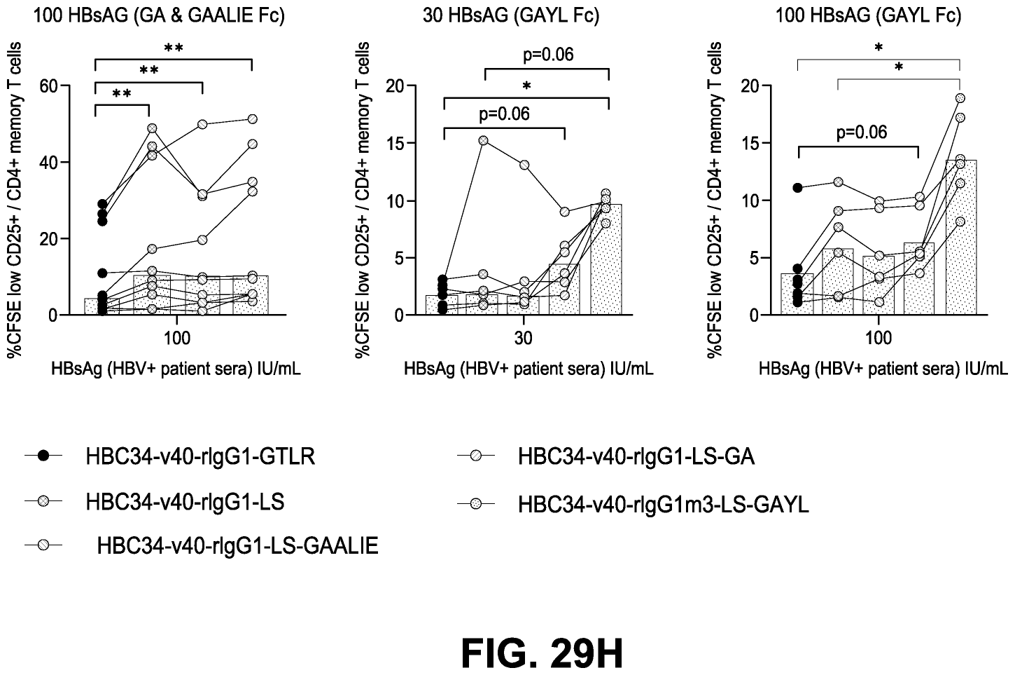

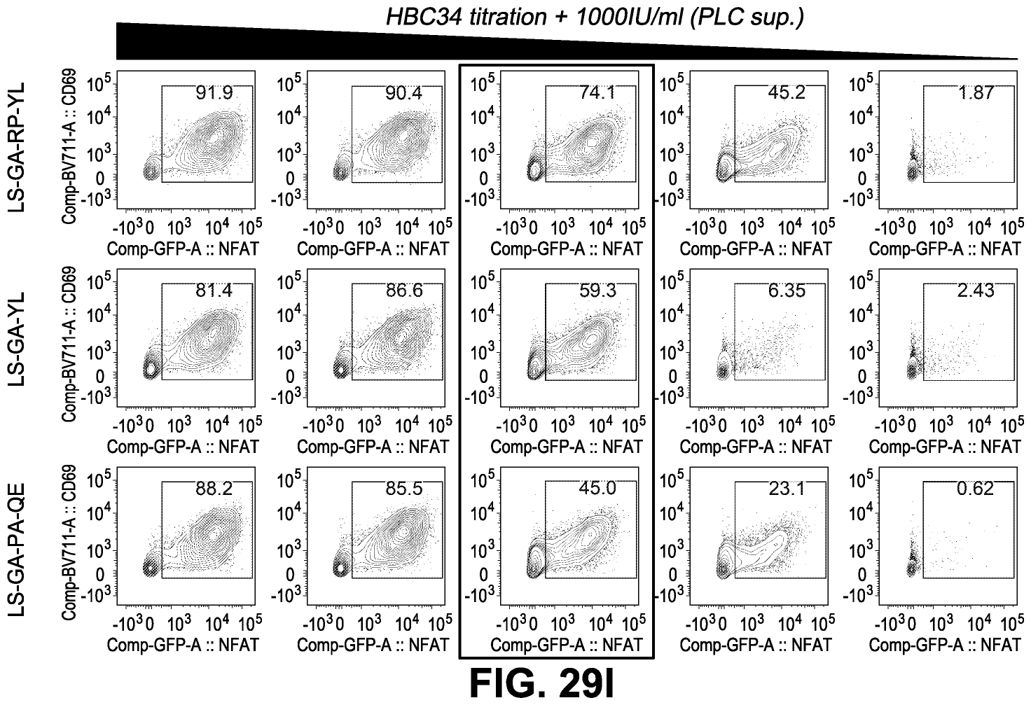

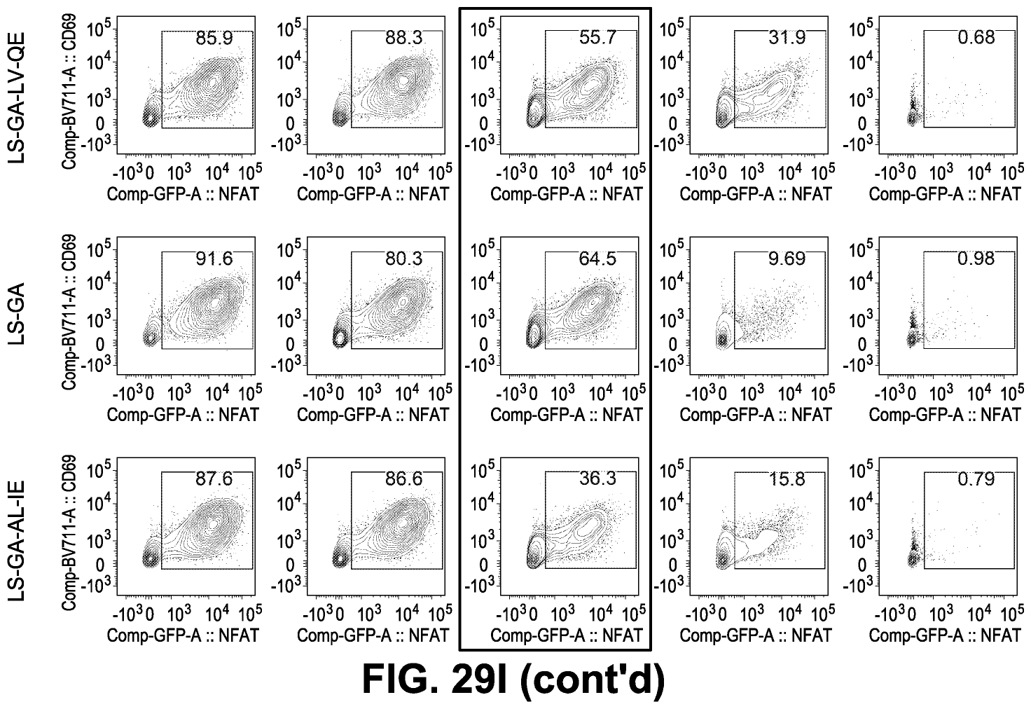

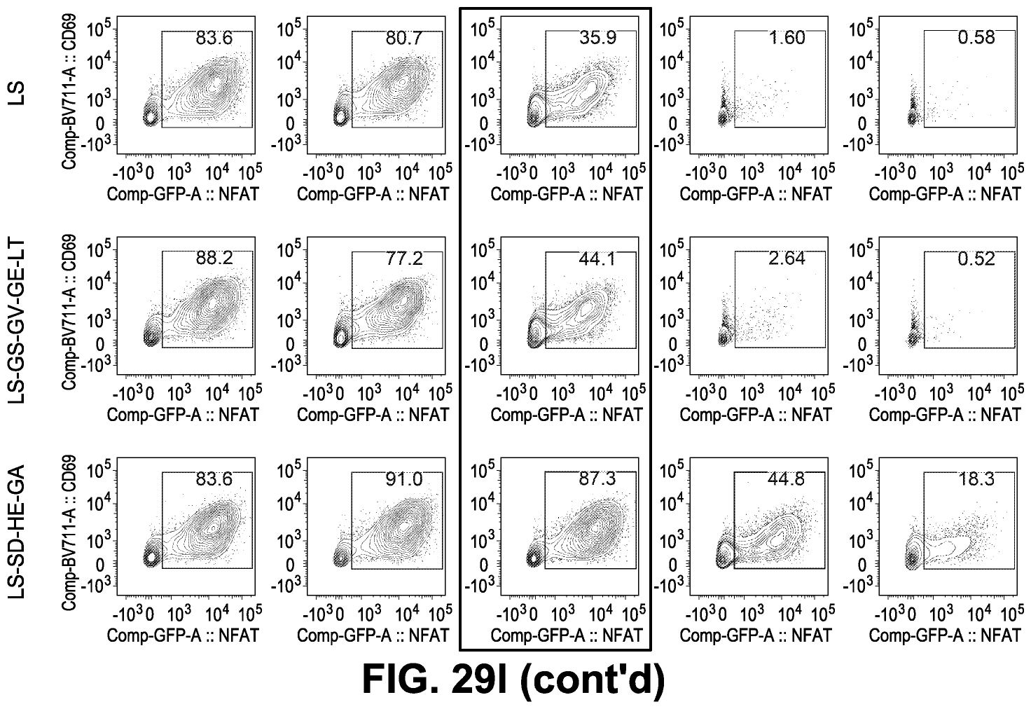

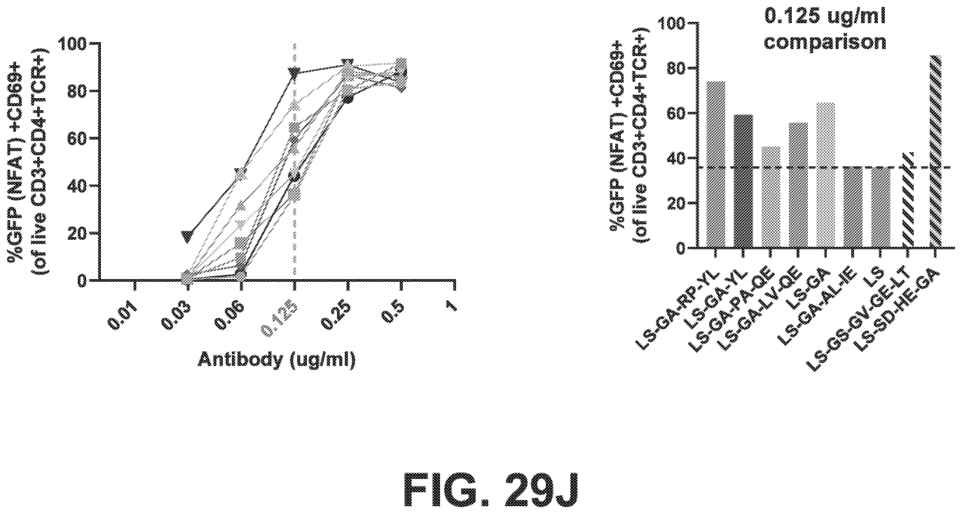

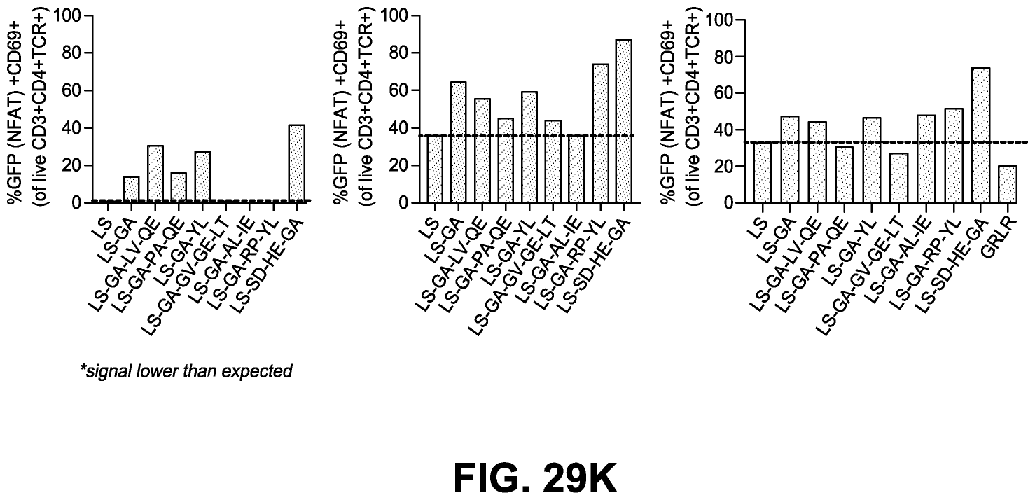

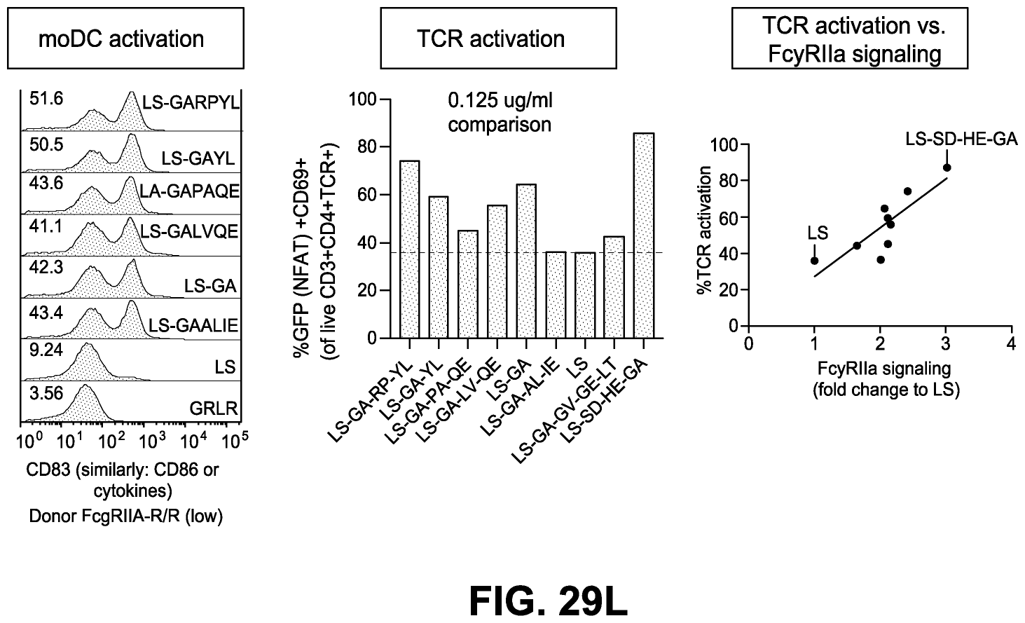

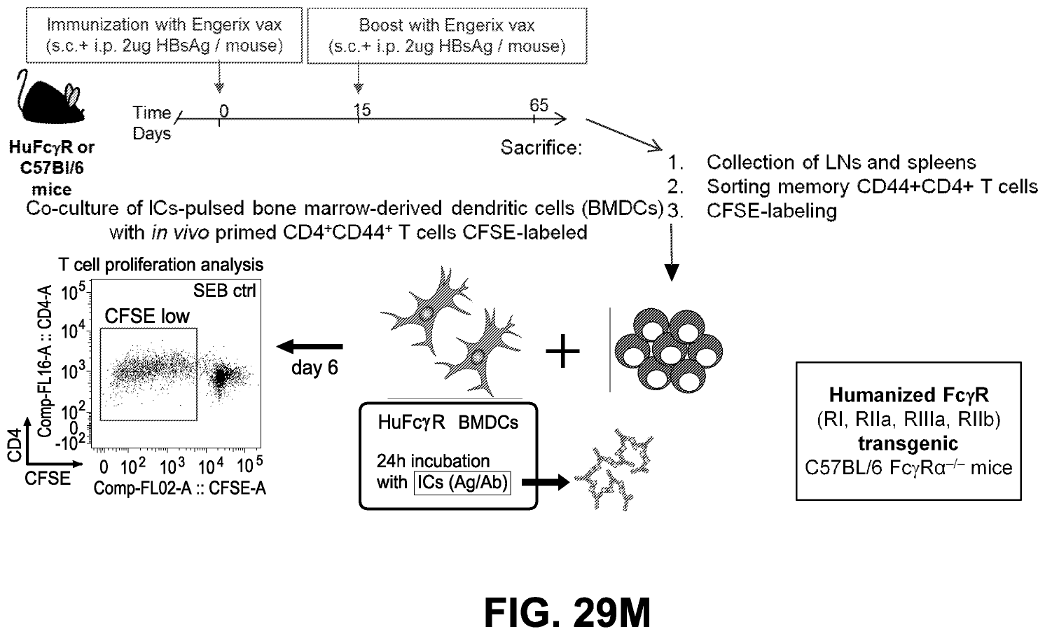

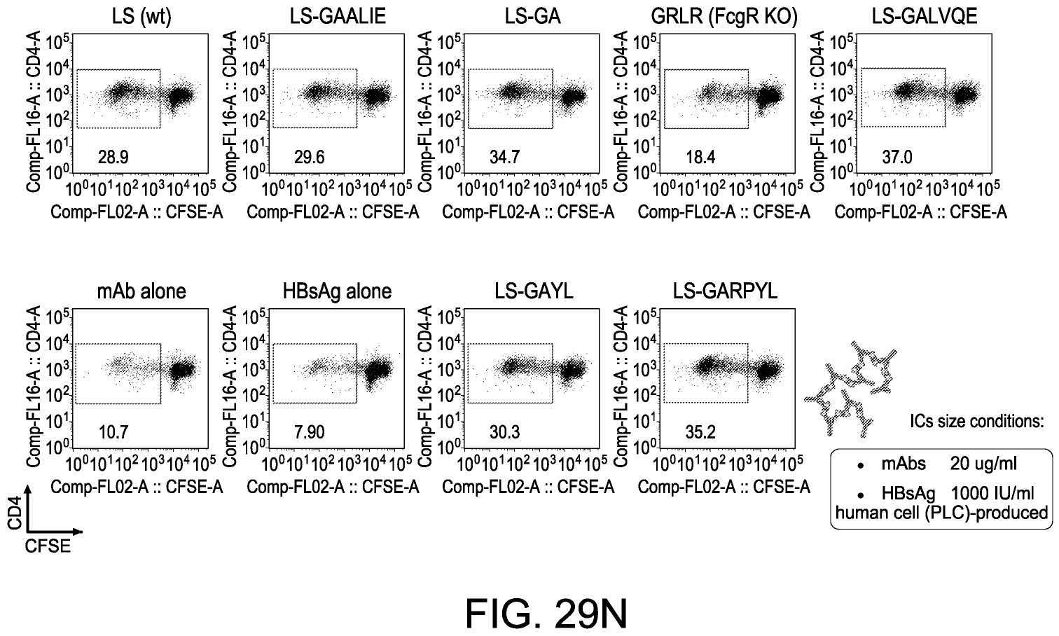

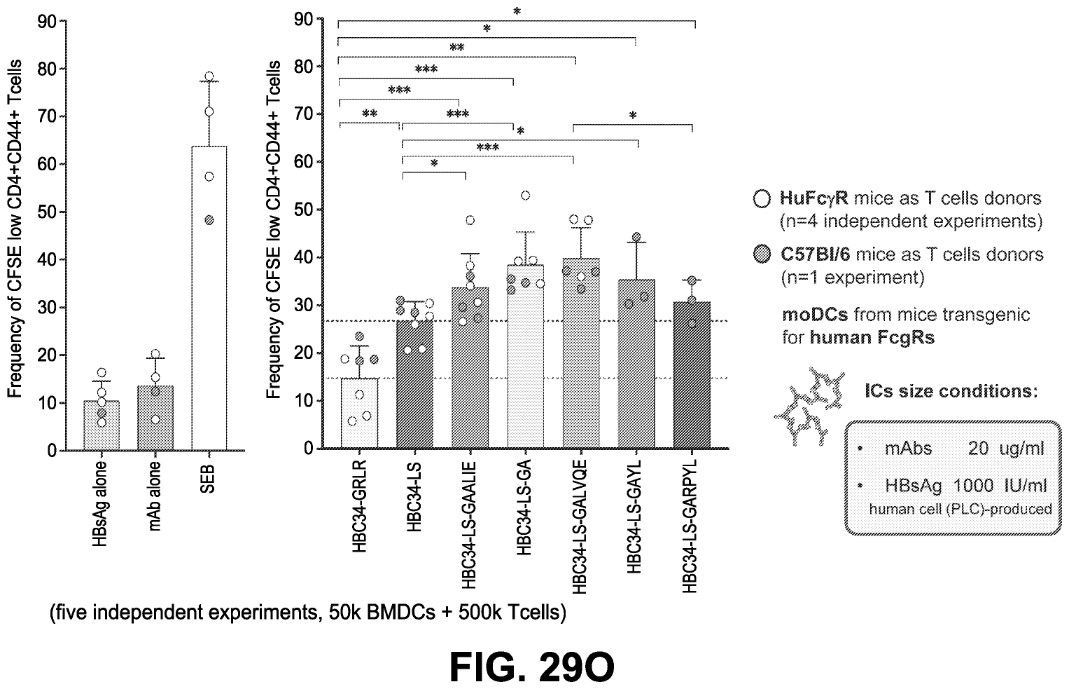

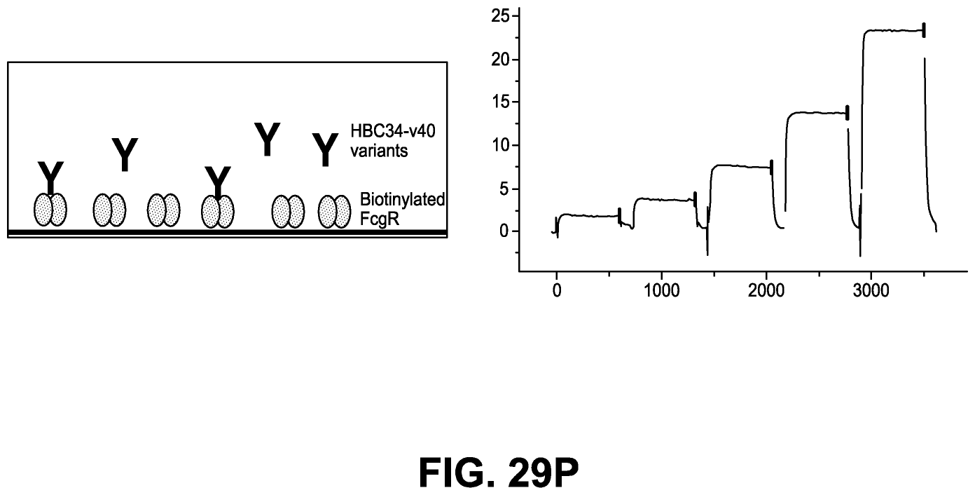

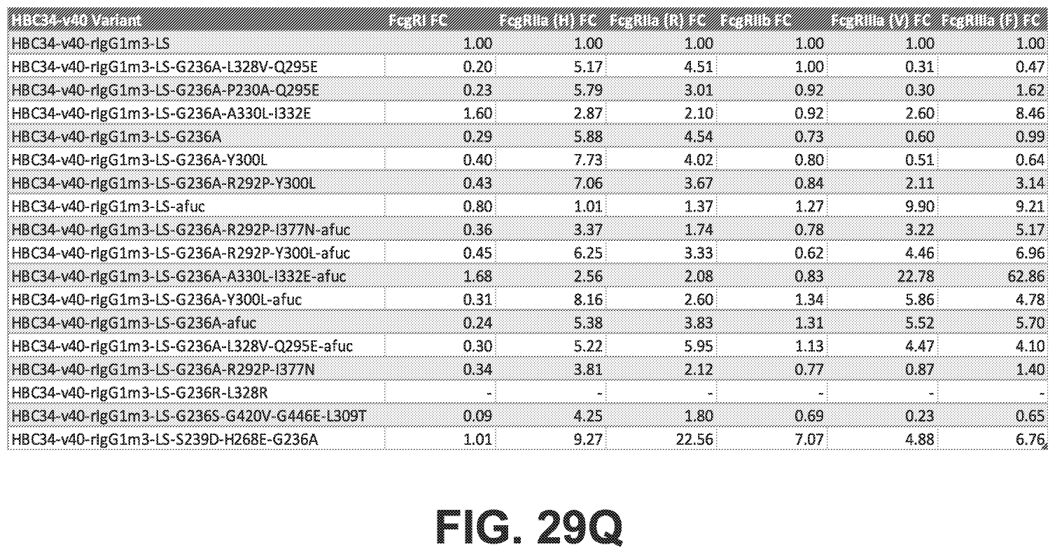

Further studies were performed as shown and described in FIGS. 21-29Q. FIGS. 21-24 show FcγR activation and binding by anti-influenza (HA) antibody “FY1” Fc variants. FIGS. 25A-28D show FcγR activation and specific lysis for anti-SARS-CoV-2 antibodies (S309 and S2X259, and V-region variants of these) with variant Fc. FIGS. 29A-29R relate to anti-HBsAg (HBC34-v40) Fc variant antibodies and show: activation of and cytokine production by human monocyte-derived dendritic cells (moDCs) using antibodies and HBsAg; activation of human HBsAg-specific CD4+ memory T cells; activation of HBsAg-specific TCR-transgenic Jurkat reporter CD4+ T cells; re-stimulation of CD4+ memory T cells from HBV-vaccinated huFcγR mice by antibody:HBsAg immune complexes (ICs); and binding kinetics (including fold-change vs. control Fc) of HBC34-v40 Fc variants for human FcγRs.

The various embodiments described above can be combined to provide further embodiments. All of the U.S. patents, U.S. patent application publications, U.S. patent applications, foreign patents, foreign patent applications and non-patent publications referred to in this specification, including U.S. Provisional Application No. 63/192,549, filed May 24, 2021, U.S. Provisional Application No. 63/265,032, filed Dec. 6, 2021, and U.S. Provisional Application No. 63/266,453, filed on Jan. 5, 2022, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A polypeptide comprising a variant of: (i) an IgG CH2 polypeptide or (ii) an IgG Fc polypeptide or a fragment thereof,
wherein the variant comprises an alanine (A) at EU position 236 and a leucine (L) at EU position 300.

2. The polypeptide of claim 1, wherein the variant, and optionally the polypeptide, has increased binding to a human FcγRIIa
as compared to the binding of a reference polypeptide to the human FcγRIIa,
wherein, optionally, binding is as determined using an electrochemiluminescence assay, further optionally Meso Scale Discovery.

3. (canceled)

4. The polypeptide of claim 2, wherein the human FcγRIIa comprises:
i) H131 and, optionally, the increased binding to the human FcγRIIa H131 comprises at least 4-fold, at least 5-fold, at least 6-fold, at least 7-fold, at least 8-fold, at least 9-fold, or at least 10-fold, at least 11-fold, at least 12-fold, at least 13-fold, at least 14-fold, at least 15-fold, at least 16-fold, at least 17-fold, or at least 18-fold greater binding to the human FcγRIIa H131 as compared to the binding of a reference polypeptide comprising a wild-type human IgG Fc polypeptide or a fragment thereof to the human FcγRIIa H131; or
ii) R131 and, optionally, the increased binding to the human FcγRIIa R131 comprises at least 4-fold greater binding to the human FcγRIIa R131 as compared to the binding of a reference polypeptide comprising a wild-type human IgG Fc polypeptide or a fragment thereof to the human FcγRIIa R131.

5.-9. (canceled)

10. The polypeptide of claim 1, further comprising a proline (P) at EU position 292.

11.-24. (canceled)

25. A polypeptide comprising a variant of: (i) an IgG CH2 polypeptide or (ii) an IgG Fc polypeptide or a fragment thereof,
wherein the variant comprises an alanine (A) at EU position 236, a proline (P) at EU position 292, and a leucine (L) at EU position 300.

26.-61. (canceled)

62. The polypeptide of claim 1, wherein the variant further comprises one or more modification that enhances or further enhances binding to a human FcRn
as compared to (1) a reference polypeptide that comprises a wild-type human IgG1 Fc polypeptide and/or to (2) the polypeptide claim 1 without the one or more modification.

63. The polypeptide of claim 62, wherein the one or more modification that enhances binding to the human FcRn comprises the amino acid substitutions:
(i) M428L/N434S;
(ii) M252Y/S254T/T256E;
(iii) T250Q/M428L;
(iv) P257I/Q311I;
(v) P257I/N434H;
(vi) D376V/N434H;
(vii) T307A/E380A/N434A;
(viii) N434A;
(ix) M428L/N434A; or
(x) any combination of (i)-(ix).

64. The polypeptide of claim 1, wherein the variant does not comprise any additional mutations as compared to the reference IgG Fc polypeptide or fragment thereof, the IgG CH2 polypeptide, the IgG hinge-CH2 polypeptide, or the IgG hinge-Fc polypeptide or fragment thereof, respectively.

65. The polypeptide of claim 1, which comprises a Fc polypeptide.

66. The polypeptide of claim 1, which is a monomer comprised in a polypeptide dimer (e.g., a Fc dimer).

67. The polypeptide of claim 1,
i)which is a monomer comprised in a polypeptide homodimer (e.g., a Fc homodimer); or
ii) which is a monomer comprised in a polypeptide heterodimer (e.g., a Fc heterodimer, optionally comprising a protuberance in a first Fc of the heterodimer and a corresponding cavity in a second Fc of the heterodimer, and/or comprising one or more mutations that provide or contribute to an opposite charge in each of the two Fc monomers (e.g., a positive charge in a region of a first monomer and a negative charge in a corresponding region of a second monomer), and/or comprising a heterologous amino acid sequence in one or both monomers, to promote dimerization of the two Fc monomers).

68. (canceled)

69. The polypeptide of claim 1, which is comprised in an antibody.

70. An antibody comprising the polypeptide of claim 1.

71. An antibody comprising a variant of an IgG Fc, wherein the variant comprises an alanine (A) at EU position 236 and a leucine (L) at EU position 300.

72.-76. (canceled)

77. An antibody comprising a variant of an IgG Fc, wherein the variant comprises an alanine (A) at EU position 236, a proline (P) at EU position 292, and a leucine (L) at EU position 300.

78.-86. (canceled)

87. The polypeptide of claim 1, wherein the variant is derived from or comprises:
i) an IgG1 isotype, an IgG2 isotype, an IgG3 isotype, or an IgG4 isotype;
ii) a human IgG1 isotype, a human IgG2 isotype, a human IgG3 isotype, or a human IgG4 isotype; or
iii) a human IgG1 isotype, optionally comprising a Glm3 allotype, a Glm17 allotype, a Glm3,1 allotype, or a Glm17,1 allotype.

88. The polypeptide of claim 1, wherein the variant is derived from or comprises a human Fc or a fragment thereof, or from a human antibody heavy chain or a fragment thereof.

89.-92. (canceled)

93. The antibody of claim 70, wherein the variant further comprises one or more modification that enhances binding to a human FcRn
as compared to (1) a reference antibody comprises a wild-type human IgG1 Fc polypeptide and/or to (2) the antibody of claim 70 without the one or more modification,
wherein the one or more modification that enhances binding to the human FcRn comprises the amino acid substitutions:
(i) M428L/N434S;
(ii) M252Y/S254T/T256E;
(iii) T250Q/M428L;
(iv) P257I/Q311I;
(v) P257I/N434H;
(vi) D376V/N434H;
(vii) T307A/E380A/N434A;
(viii) M428L/N434A; or
(ix) any combination of (i)-(viii).

94. (canceled)

95. The antibody of claim 70, wherein the variant does not comprise any additional mutations as compared to a reference wild-type IgG Fc.

96. The antibody of claim 69, wherein the antibody is capable of specifically binding to:
(i) a target (e.g., an antigen) that is expressed or produced by a pathogen (e.g., virus, bacterium, parasite, fungus) or by a cell infected with the pathogen, wherein, optionally, the pathogen comprises a virus and the virus comprises: a coronavirus; a betacoronavirus; a sarbecovirus; an embecovirus; a nobecovirus; a merbecovirus; a metapneumovirus; a hibecovirus; a SARS-CoV-2; a hepatitis B virus; a hepatitis D virus; an influenza A virus; a cytomegalovirus; a rhinovirus; a hepatitis C virus; an influenza B virus; a human immunodeficiency virus; a respiratory virus; a respiratory syncytial virus; a zika virus; a rabies virus; a dengue virus; a flavivirus; an ebolavirus; or any combination thereof,
(ii) a target (e.g., an antigen) that is expressed by, and/or is expressed on a cell surface of, a tumor cell, optionally a cancer cell or a cell of a proliferative or hyperproliferative disorder;
(iii) a target (e.g., an antigen) that is associated with an autoimmune disease;
(iv) a target (e.g., an antigen) that is associated with a neurodegenerative disease
(v) an immune system signaling molecule, such as a cytokine;
(vi) a target (e.g., an antigen) that is associated with inflammation;
(vii) a target (e.g., an antigen) that is associated with a non-infectious disease; or
(viii) any combination of (i)-(vii).

97. The antibody of claim 69, wherein:
i) the antibody comprises a chimeric antibody, a humanized antibody, a neutralizing antibody, a human antibody, an IgNAR, a camelid nanobody, or any combination thereof; and/or
ii) the antibody is a multispecific antibody, such as a bispecific antibody, a trispecific antibody, or a tetraspecific antibody.

98. (canceled)

99. The antibody of claim 69, wherein the antibody is comprised in an antibody conjugate.

100. The polypeptide of claim 1, the wherein the polypeptide or the Fc polypeptide: (1) comprises a Fc fusion protein; and/or (2) comprises an Fcab.

101. The polypeptide of claim 100, wherein the Fc fusion protein further comprises:
(i) a receptor domain (e.g. an ectodomain of a receptor protein, or a ligand-binding portion thereof);
(ii) a ligand;
(iii) a replacement protein; or
(iv) any combination of (i)-(iii).

102. The polypeptide of claim 1, which is conjugated, linked, or fused to a payload moiety.

103. The polypeptide of claim 102, wherein the payload moiety comprises: an antibody or an antigen-binding fragment thereof, a cytotoxic agent (e.g., a chemotherapeutic agent); a detectable compound or detectable label; an oligonucleotide (e.g., an antisense oligonucleotide, a siRNA, or the like); a vector; an agent that stimulates an immune response; a growth factor; or any combination thereof.

104. The polypeptide of claim 1, wherein the polypeptide:
i) is afucosylated; has been produced in a host cell that is incapable of fucosylation or that is inhibited in its ability to fucosylate a polypeptide; has been produced under conditions that inhibit fucosylation thereof by a host cell; or any combination thereof; and/or
ii) comprises an amino acid mutation that (1) inhibits fucosylation as compared to a reference polypeptide, and/or (2) that abrogates a fucosylation site that is present in the reference polypeptide.

105. (canceled)

106. A polynucleotide encoding the polypeptide of claim 1.

107. (canceled)

108. A(n e.g. expression) vector comprising the polynucleotide of claim 106.

109. A host cell comprising the polynucleotide of claim 106.

110. (canceled)

111. (canceled)

112. A composition comprising:
the polypeptide of claim 1,
and a pharmaceutically acceptable carrier, excipient, or diluent.

113. A method of treating or preventing an infectious disease (optionally caused by a viral, bacterial, fungal, or parasitic infection), a cancer, a proliferative disorder, a neurodegenerative disease, an autoimmune disease, or any combination thereof in a subject, the method comprising administering to the subject an effective amount of
the polypeptide of claim 1.

114.-117. (canceled)

118. The polypeptide of claim 1, wherein the polypeptide is capable of specifically binding to human immunodeficiency virus.

119. The polypeptide of claim 1, comprising the amino acid sequence of SEQ ID NO:11.

120. The polypeptide of claim 10, comprising the amino acid sequence of SEQ ID NO:12.


SEQUENCE LISTING (48 sequences)

SEQ ID NO: 1 - protein, 330 aa, Homo sapiens [sequence omitted]

SEQ ID NO: 2 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 4 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 125 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 6 - protein, 217 aa, Artificial
  Synthetic sequence Human IgG1 CH2-CH3 amino acid sequence with G236A, L328V, and Q295E mutations
APELLAGPSVFLFPPKPKDTLMISRTPEVTCVVVDVSHEDPEVKFNWYVDGVEVHNAKTKPREEEYNSTYRVVSVLTVLHQDWLNGKEYKCKVSNKAVPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 232 aa, Artificial sequence
  Synthetic sequence Human IgG1 hinge-CH2-CH3 amino acid sequence with G236A, P230A, and Q295E mutations
EPKSCDKTHTCPPCAAPELLAGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 217 aa, Artificial sequence
  Synthetic sequence Human IgG1 CH2-CH3 amino acid sequence with G236A, R292P, and I377N mutations
APELLAGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDNAVEWESNGQPENNYKTTPPVLDSDGSFFLYSKLTVDKSRWQQGNVFSCSVMHEALHNHYTQKSLSLSPGK

SEQ ID NO: 9 - protein, 217 aa, Artificial sequence
  Synthetic sequence Human IgG1 CH2-CH3 amino acid sequence with G236A, K334A, and Q295E mutations
APELLAGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, Artificial sequence
  Synthetic sequence Human IgG1 CH2-CH3 amino acid sequence with G236S, R292P, and Y300L mutations
APELLSGPSVFLFPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 217 aa, Artificial sequence
  Synthetic sequence Human IgG1 CH2-CH3 amino acid sequence with G236A and Y300L mutations
APELLAGPSVFLFPPKPKDTLMISRTPEVTCVVVDVSHEDPEVKFNWYVDGVEVHNAKTKPREEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 217 aa, Artificial sequence
  Synthetic sequence Human IgG1 CH2-CH3 amino acid sequence with G236A, R292P, and Y300L mutations
APELLAGPSVFLFPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 217 aa, Artificial sequence
  Synthetic sequence Human IgG1 CH2-CH3 amino acid sequence with G236S, G420V, G446E, and L309T mutations
APELLSGPSVFLFPPKPKDTLMISRTPEVTCVVVDVSHEDPEVKFNWYVDGVEVHNAKTKPREEQYNSTYRVVSVLTVTHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQVNVFSCSVMHEALHNHYTQKSLSLSPEK

SEQ ID NO: 14 - protein, 217 aa, Artificial sequence
  Synthetic sequence Human IgG1 CH2-CH3 amino acid sequence with G236A and R292P mutations
APELLAGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 217 aa, Artificial sequence
  Synthetic sequence Human IgG1 CH2-CH3 amino acid sequence with R292P and Y300L mutations
APELLGGPSVFLFPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 217 aa, Artificial sequence; identical to SEQ ID NO: 14
  Synthetic sequence Human IgG1 CH2-CH3 amino acid sequence with G236A and R292P mutations

SEQ ID NO: 17 - protein, 217 aa, Artificial sequence
  Synthetic sequence Human IgG1 CH2-CH3 amino acid sequence with Y300L mutation
APELLGGPSVFLFPPKPKDTLMISRTPEVTCVVVDVSHEDPEVKFNWYVDGVEVHNAKTKPREEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 217 aa, Artificial sequence
  Synthetic sequence Human IgG1 CH2-CH3 amino acid sequence with E345K, G236S, L235Y, and S267E mutations
APELYSGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPRKPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 232 aa, Artificial sequence
  Synthetic sequence Human IgG1 hinge-CH2-CH3 amino acid sequence with E272R, L309T, S219Y, and S267E mutations
EPKYCDKTHTCPPCPAPELLGGPSVFLFPPKPKDTLMISRTPEVTCVVVDVEHEDPRVKFNWYVDGVEVHNAKTKPREEQYNSTYRVVSVLTVTHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 217 aa, Artificial sequence
  Synthetic sequence Human IgG1 CH2-CH3 amino acid sequence with G236Y mutation
APELLYGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 217 aa, Artificial sequence
  Synthetic sequence Human IgG1 CH2-CH3 amino acid sequence with G236W mutation
APELLWGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 217 aa, Artificial sequence
  Synthetic sequence Human IgG1 CH2-CH3 amino acid sequence with F243L, G446E, P396L, and S267E mutations
APELLGGPSVFLLPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPEK

SEQ ID NO: 23 - protein, 217 aa, Artificial sequence
  Synthetic sequence Human IgG1 CH2-CH3 amino acid sequence with G236A mutation
APELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24-25 - skipped (2 entries)

SEQ ID NO: 26 - protein, 119 aa, Artificial sequence
  Synthetic sequence HBC34-v35 VH amino acid sequence
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSS

SEQ ID NO: 27 - protein, 106 aa, Artificial sequence
  Synthetic sequence HBC34-v35 VL amino acid sequence
SYELTQPPSVSVSPGQTVSIPCSGDKLGNKNVAWFQHKPGQSPVLVIYEVKYRPSGIPERFSGSNSGNTATLTISGTQAMDEAAYFCQTFDSTTVVFGGGTRLTVL

SEQ ID NO: 28 - protein, 127 aa, Artificial sequence [sequence omitted]

SEQ ID NO: 29 - protein, 107 aa, Artificial sequence [sequence omitted]

SEQ ID NO: 30 - protein, 126 aa, Artificial sequence
  Synthetic sequence S2X259 VH amino acid sequence
QVQLVQSGAEVKKPGSSVKVSCKASGGIFNTYTISWVRQAPGQGLEWMGRIILMSGMANYAQKIQGRVTITADKSTSTAYMELTSLRSDDTAVYYCARGFNGNYYGWGDDDAFDISGQGTLVTVYS

SEQ ID NO: 31 - protein, 113 aa, Artificial sequence
  Synthetic sequence S2X259 VL amino acid sequence
QTVLTQPPSVSGAPGQRVTISCTGSNSNIGAGYDVHWYQQLPGTAPKLLICGNSNRPSGVPDRFSGSKSGTSASLAITGLQAEDEADYYCQSYDSSLSGPNWVFGGGTKLTVL

SEQ ID NO: 32 - protein, 126 aa, Artificial sequence
  Synthetic sequence  S2X259-v5  VH amino acid sequence
QVQLVQSGAEVKKPGSSVKVSCKASGGIFNTYTISWVRQAPGQGLEWMGRIILMSGMANYAQKIQGRVTITADKSTSTAYMELTSLRSDDTAVYYCARGFNGNYYGWGDDDAFDIWGQGTLVTVSS

SEQ ID NO: 33 - protein, 113 aa, Artificial sequence
  Synthetic sequence S2X259-variant VL amino acid sequence
QTVLTQPPSVSGAPGQRVTISCTGSNSNIGAGYDVHWYQQLPGTAPKLLIYGNSNRPSGVPDRFSGSKSGTSASLAITGLQAEDEADYYCQSYDSSLSGPNWVFGGGTKLTVL

SEQ ID NO: 34 - protein, 128 aa, Artificial sequence
  Synthetic sequence FM08 VH amino acid sequence
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSS

SEQ ID NO: 35 - protein, 103 aa, Artificial sequence
  Synthetic sequence FM08 VL amino acid sequence
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIK

SEQ ID NO: 36 - protein, 330 aa, Artificial sequence
  Synthetic sequence IgG1m3 CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 330 aa, Artificial sequence [sequence omitted]

SEQ ID NO: 38 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 119 aa, Artificial sequence; identical to SEQ ID NO: 26
  Synthetic sequence HBC34-v40 VH amino acid sequence

SEQ ID NO: 40 - protein, 106 aa, Artificial sequence
  Synthetic sequence HBC34-v40 VL
SYELTQPPSVSVSPGQTVSIPCSGDKLGNKNVAWFQHKPGQSPVLVIYQDSYRPSGIPERFSGSNSGNTATLTISGTQAMDEAAYFCQTFDSTTVVFGGGTRLTVL

SEQ ID NO: 41 - protein, 126 aa, Artificial sequence
  Synthetic sequence S2X259.1-v3.2 VH
QVQLVQSGAEVKKPGSSVKVSCKASGGIDQTYTISWVRQAPGQGLEWMGRIILISGRADYAQKIQGRVTITADKSTSTAYMELTSLRSDDTAVYYCARGFNANYYGWGDDDAFDIWGQGTLVTVSS

SEQ ID NO: 42 - protein, 113 aa, Artificial sequence
  Synthetic sequence S2X259.1-v3.2 VL
QTVLTQPPSVSGAPGQRVTISCTGSNSNIGAGYDVHWYQQLPGTAPKLLIVGQSNRPSGVPDRFSGSKSGTSASLAITGLQAEDEADYYCQSYDSSGSAPNWVFGGGTKLTVL

SEQ ID NO: 43 - protein, 127 aa, Artificial sequence
  Synthetic sequence Sotrovimab VH
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 44 - protein, 107 aa, Artificial sequence [sequence omitted]

SEQ ID NO: 45 - protein, 217 aa, Artificial sequence
  Synthetic sequence Human IgG1 CH2-CH3 amino acid sequence with G236A, S239D, and H268E mutations
APELLAGPDVFLFPPKPKDTLMISRTPEVTCVVVDVSEEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 113 aa, Artificial sequence
  Synthetic sequence S2X259-v5 VL
QTVLTQPPSVSGAPGQRVTISCTGSNSNIGAGYDVHWYQQLPGTAPKLLIVGNSNRPSGVPDRFSGSKSGTSASLAITGLQAEDEADYYCQSYDSSLSGPNWVFGGGTKLTVL

SEQ ID NO: 47 - protein, 126 aa, Artificial sequence
  Synthetic sequence S2X259.1 VH
QVQLVQSGAEVKKPGSSVKVSCKASGGIDNTYTISWVRQAPGQGLEWMGRIILISGRADYAQKIQGRVTITADKSTSTAYMELTSLRSDDTAVYYCARGFNGNYYGWGDDDAFDIWGQGTLVTVSS

SEQ ID NO: 48 - protein, 113 aa, Artificial sequence
  Synthetic sequence S2X259.1 VL
QTVLTQPPSVSGAPGQRVTISCTGSNSNIGAGYDVHWYQQLPGTAPKLLIVGNSNRPSGVPDRFSGSKSGTSASLAITGLQAEDEADYYCQSYDSSGSAPNWVFGGGTKLTVL